(19) United States
(12) Patent Application Publication
(10) Pub. No.: US 2012/0237496 A1
(43) Pub. Date: Sep. 20, 2012

(54) PROTEASE VARIANTS

(76) Inventors: Joerg Birkenfeld, Duesseldorf (DE); Andrea Eicker, Moenchengladbach (DE); Per-Ola Freskgard, Sodertalje (SE); Claudia Gotzberger-Schad, Leichlingen (DE); Joanna Grudzinska, Bonn (DE); Ulrich Haupts, Odenthal (DE); Josi Innig, Bergisch Gladbach (DE); Christoph Mahlert, Koln (DE); Andreas Scheidig, Koln (DE); Michael Strerath, Koln (DE); Jan Tebbe, Koln (DE); Johan Per-Wallin, Sodertalje (SE); Nina Wobst, Wuppertal (DE); Carl Innes Webster, Cambridge (GB); Lutz Jermutus, Cambridge (GB)

(21) Appl. No.: 13/378,764

(22) PCT Filed: Jun. 21, 2010
(86) PCT No.: PCT/US2010/039392

§ 371 Date: May 14, 2012

Related U.S. Application Data
(60) Provisional application No. 61/218,733, filed on Jun. 19, 2009.
(60) Provisional application No. 61/244,515, filed on Sep. 22, 2009.

(51) Int. Cl. A61K 38/48; C12N 9/96; C12N 5/10; A61P 25/28; C12N 1/19; C12N 15/63; C12N 15/57; C12N 9/64; C12N 1/15

(52) U.S. Cl. 424/94.67; 435/226; 435/188; 435/358; 435/365; 435/355; 435/254.11; 435/254.2; 435/254.21; 435/320.1; 536/23.2

(57) ABSTRACT

The present invention relates to polypeptides comprising protease variants of wild type human neprilysin having an altered specificity and/or activity. In particular the present invention relates to polypeptides comprising protease variants derived from human neprilysin having an increased specificity and/or activity against certain substrates, in particular against amyloid beta.

The present invention relates to nucleic acid and amino acid sequences of variants of human neprilysin with altered substrate specificity relative to wild-type human neprilysin and use of such variants in pharmaceutical compositions. In particular, the present invention relates to neprilysin variant polypeptides with increased specificity for cleavage of amyloid beta (Aβ) peptides compared to wild-type neprilysin. The invention also relates to fusion proteins comprising such neprilysin variant molecules. Polypeptides comprising neprilysin variants may be used in the treatment of diseases associated with accumulation of amyloid beta, in particular Alzheimer's disease.

### BACKGROUND OF THE INVENTION

Engineered proteases are desirable as therapeutics because the cleavage of a substrate peptide or protein associated with a disease will often lead to its irreversible inactivation or activation. However, for use as a drug, a protease must have a sufficient activity on the target, but must not cleave other substrates to an extent that leads to unacceptable toxic side effects under treatment conditions.

The specificity of proteases, i.e. their ability to recognize and hydrolyze preferentially certain peptide substrates, can be expressed qualitatively and quantitatively. Qualitatively, proteases that act on one or a small number of peptides have a high specificity, whereas proteases that act on many different peptides are deemed to have low specificity. In quantitative terms, the specificity profile of a protease is given by the respective kcat/Km ratios for all substrates, including potentially kcat/Km ratios for several cleavage sites in a given substrate. Modern methods of protein engineering permit modulation of the specificity of a given protease, potentially enabling the generation of proteases with desired specificities for use as prophylactic or therapeutic protein drugs.

An accumulation or increase in the activity of a polypeptide compared to the “normal” level may contribute to the cause or symptoms of a disease; in such cases the inactivation of the polypeptide by proteolytic cleavage may be beneficial for the patient. Many different polypeptides can be envisioned as targets for proteolytic inactivation. These include small peptides such as bioactive peptides of the endocrine system, for example involved in the regulation of vasoactivity, pain, appetite, cardiac function, immune functions, metabolic regulation, circadian rhythm and others. Other examples include small and large proteins or homo- and heteromeric multiprotein complexes such as soluble and membrane bound proteins and receptors, structural proteins, cytokines, enzymes, antibodies, transporters and others. Many peptides are known to have potent regulatory functions including Angiotensin-1 and -2, ANP, BNP, bradykinin, Endothelin-1 and -2, Neuropeptide Y and Neurotensin, as well as Adrenomedullin, Bombesin, BLP, CGRP, Enkephalins, FGF-2, fMLP, GRP, Neurokinins, Neuromedin C, Oxytocin, PAMP, Substance P, VIP and others. Increased activity of any of these may lead to undesirable effects in a patient. For example, neurotensin stimulates the proliferation of prostate cancer PC3 cells (Carraway et al. (2007) Regul Pept. 141(1-3):140-53) and its degradation in vivo may mitigate disease. Bradykinin is involved in blood pressure regulation, but also in neuropathic pain and cardiac remodeling. As will be shown, protease variants with increased specificity towards neurotensin or bradykinin can be generated.

Alzheimer's disease (AD) is a progressive neurodegenerative disorder characterized by a loss of neurons in discrete regions of the brain, particularly in the cortex and hippocampus. The neuropathological hallmarks that occur in the brains of individuals suffering from AD are senile plaques and profound cytoskeletal changes coinciding with the appearance of abnormal filamentous structures. The neuronal loss is accompanied by extracellular deposition of amyloid beta (Aβ) peptides in the form of senile plaques and intracellular accumulation of neurofibrillary tangles made of a hyperphosphorylated form of the microtubule-associated protein tau. Both familial and sporadic cases share the deposition in brain of extracellular, fibrillary β-amyloid as a common pathological hallmark that is believed to be associated with impairment of neuronal functions and neuronal loss (Younkin S. G., Ann. Neurol. 37, 287-288, 1995; Selkoe, D. J., Nature 399, A23-A31, 1999; Borchelt D. R. et al., Neuron 17, 1005-1013, 1996). β-amyloid deposits are composed of several species of amyloid-β peptides (Aβ; especially Aβ₁₋₄₂, which is deposited progressively in amyloid plaques. Genetic evidence suggests that increased amounts of Aβ₁₋₄₂ are produced in many, if not all, genetic conditions that cause familial AD (Borchelt D. R. et al., Neuron 17, 1005-1013, 1996; Duff K. et al., Nature 383, 710-713, 1996; Scheuner D. et al., Nat. Med. 2, 864-870, 1996; Citron M. et al., Neurobiol. Dis. 5, 107-116, 1998), suggesting that amyloid formation may be caused either by increased generation of Aβ₁₋₄₂, or decreased degradation, or both (Glabe, C., Nat. Med. 6, 133-134, 2000).

Currently, there is no cure for AD. However, Aβ has become a major target for the development of drugs, both with the aim to reduce formation (Vassar, R. et al., Science 286, 735-41, 1999), and to activate mechanisms that accelerate its clearance from brain. Although considerable effort has focused on reducing the generation of Aβ, considerably less emphasis has been placed on the clearance of these peptides.

Bard et al. (Nature Medicine, Vol. 6, Number 8, 916-919, 2000) report that peripheral administration of antibodies against Aβ is sufficient to reduce amyloid burden. The passively administered antibodies were able to cross the blood-brain barrier and enter the central nervous system, bind to (“decorate”) plaques and induce clearance of pre-existing amyloid. However, even a passive immunisation against Aβ may cause undesirable side effects in human patients.

DeMattos (PNAS 98: 8850-8855, 2001) have described the sink hypothesis, which states that Aβ peptides can be removed from CNS indirectly by lowering the concentration of the peptides in the plasma. De Mattos used an antibody that binds Aβ in the plasma. By preventing influx of Aβ from the plasma to CNS and/or changing the equilibrium between the plasma and CNS (due to a lowering of the free Aβ concentration in plasma) Aβ is sequestered from the CNS. Two other Aβ binding agents, gelsolin and GM1, unrelated to antibodies, have also been shown through binding in plasma to be effective in removing Aβ from CNS and reducing or preventing brain amyloidosis (Matsuoka et al. (J. Neuroscience 23: 29-33, 2003).

An alternative approach to remove Aβ is to use an enzyme that degrades Aβ into smaller fragments that have lower toxicological effects and are more readily cleared. It is postulated that this enzymatic digestion of the Aβ will also work through the sink hypothesis mechanism by lowering the free concentration of Aβ in plasma. However, this approach also provides a possibility of direct clearance of Aβ in the CNS and/or CSF. This approach will not only lower the free concentration of Aβ but also directly clear the full-length peptide from the environment. This approach is advantageous because it will not increase the total (free and bound) concentration of Aβ in the plasma as has been seen in cases when using Aβ peptide binding agents such as antibodies. Neprilysin is an enzyme described in the literature that degrades the Aβ-peptide at multiple cleavage sites generating small fragments that are cleared from the blood stream easily (Leissring et al., JBC. 278: 37314-37320, 2003). Neprilysin has also been reported to play a key role in regulating the level of Aβ peptide in the brain. Evidence suggests that down-regulation of neprilysin at the early stages of AD development, accompanied with aging, genetic deficiency (knockout), or treatment with neprilysin inhibitors, results in increasing accumulation of Aβ peptide in the brain leading to memory impairment. Conversely, overexpression of neprilysin, leads to a reduction of plaque accumulation in the brain of transgenic AD mice.

Several other proteases have been described that degrade Aβ peptide including insulin degrading enzyme, plasmin, ACE and others.

Anti-Aβ peptide antibodies have been applied to effectively reduce free Aβ levels in the blood leading to a decreased plaque deposition in the brain. However, systemic application of proteases that degrade and inactivate Aβ peptide may be an alternative; but such protease would need to be sufficiently specific Aβ peptide to be effective and to avoid induction of toxic side effects due to off-target activity.

Human neprilysin (also termed NEP, neutral endopeptidase, CD10, common acute lymphoblastic leukemia antigen (CALLA), enkephalinase; SwissProt accession P08473) is a 94 kD, type two membrane-bound Zn-metallopeptidase composed of 750 or 749 residues due to the removal of the initial methionine (SEQ ID NO:1). The 749 aa nomenclature (pdb numbering) will be used throughout this text. It is present in peptidergic neurons in the CNS, and its expression in brain is regulated in a cell-specific manner (Rogues B. P. et al., Pharmacol. Rev. 45, 87-146, 1993; Lu B. et al., J. Exp. Med. 181, 2271-2275, 1995; Lu B. et al., Ann. N.Y. Acad. Sci. 780, 156-163, 1996). The proteolytic domain (extracellular catalytic domain, ECD) comprises aa 51 to 749 and contains an active site containing a zinc-binding motif (HEXXH). A soluble form lacking the transmembrane and intracellular domains is known to be present in the circulation. Neprilysin is capable of degrading a number of peptidic substrates, including monomeric and (possibly) oligomeric forms of Aβ peptides and can act as an endopeptidase as well as a carboxypeptidase, although the relevance of these different activities under physiological conditions has not been determined in detail. Peptides that are degraded include, but are not limited to, (Table 1):

TABLE 1

| Substrate | site | Kcat [s−1] | Km [M] | Kcat/Km | Lit. |
|---|---|---|---|---|---|
| Aβ₁₋₄₀-peptide | G9-Y10 | 1.5 | 1.3 × 10⁻⁵ | 1.1 × 10⁵ | [1] |
| Aβ₁₋₄₂-peptide | G9-Y10 | | 6.95 × 10⁻⁶ | | [2] |
| Angiotensin | H9-L10 | 34 | 5.5 × 10⁻⁵ | 6.18 × 10⁵ | [3] |
| Angiotensin | P7-F8 | 42 | 1.1 × 10⁻³ | 3.78 × 10⁴ | [3] |
| Enkephalin | G3-F4 | 28 | 6.6 × 10⁻⁵ | 4.24 × 10⁵ | [4] |
| ANP | C7-F8 | | | | [5] |
| Substance P | | 84 | 1.9 × 10⁻⁴ | 4.4 × 10⁵ | [6] |
| bradykinin | | 106 | 16 × 10⁻⁵ | 6.5 × 10⁵ | [6] |

[1] Leissring et al. (2003) JBC 278: 37314-20
[2] Shirotani et al. (2001) JBC 276: 21895
[3] Rice et al. (2004) Biochem. J. 383: 45
[4] Dion et al. (1995) Biochem J. 311(2): 623-7; Marie-Claire et al. (2000) Proteins, 39: 365-71
[5] Vanneste et al. (1988) Biochem. J. 254: 531-7
[6] Brenda database

The structure of neprilysin in complex with inhibitors has been solved (Oefner et al. (2000) J. Mol. Biol. 296:341-9; Sahli et al. (2005) Helv. Chim. Acta. 88:731; PDB entries 1Y8J, 1DMT, 1R1H). Neprilysin belongs to the M13 class of metallo proteinases and is characterized by a mostly α-helical, two-domain structure. These two domains enclose an integral cavity that includes the active site. The size of the cavity limits the majority of natural substrates to <5 kDa. However, it is largely unknown which residues of neprilysin interact with the substrate and thus influence protease specificity. A few amino acids in contact with the inhibitors might be considered as part of the active site of the protease and include (Table 2):

| Site | Residue (numbering as in PDB entries) | Lit. |
|---|---|---|
| S1′ | N542 | [7] |
| | F563, F564, M579 | [9] |
| | V580, | [11] |
| | F106, A543, I558, F563, F579, V580, | [10] |
| | H583, V692, W693 | |
| S2′ | R102, | [8] |
| Active site | H583, H587, E646, Zn²⁺ | [10] |

[7] Dion et al. (1995) Biochem J. 311(2): 623-7
[8] Beaumont et al. (1992) JBC 267: 2138-41
[9] Marie-Claire et al. (2000) Proteins, 39: 365-71;
[10] Voisin et al. (2004) JBC 279: 46172-81;
[11] Vijayaraghavan et al., (1990) Biochemistry 29: 8052-8056

Neprilysin also degrades many vasoactive peptides, including bradykinin, angiotensin II, endothelin I, and the natriuretic peptides (atrial natriuretic peptide (ANP) and brain natriuretic peptide (BNP)) (Reid Ian A., Vasoactive Peptides, in “Basic and Clinical Pharmacology”, (1998), The McGraw-Hill Companies).

Angiotensin, bradykinin, endothelins and natriuretic peptides (ANP and BNP) are involved in the regulation of arterial pressure. Angiotensin II is a vasoconstrictive octapeptide. Bradykinin is a vasodilator nonapeptide. Endothelins are vasoconstrictive polypeptides of about 20 amino acids with two disulfide bridges connecting cysteine residues. ANP (28-amino acid) and BNP (32-amino acid) are vasodilator peptides synthesized in the heart and are primarily destroyed by neprilysin in kidney brush-border cells, liver, and lungs (Rademaker M. T. and Richards A.M. Clinical Science. 108, 23-36, 2005). ANP and BNP produce vasodilation and decrease blood pressure. Thus, therapeutic administration of a recombinant neprilysin molecule may shorten the half-life of natriuretic peptides and thereby aggravate hypertension or chronic heart failure.

Neprilysin also degrades some signalling peptides, including neuropeptide Y and neurotensin. Neuropeptide Y is a 36 amino acid polypeptide neurotransmitter distributed in the mammalian central nervous system. Known physiological functions within the CNS include the regulation of social and feeding behaviour, circadian rhythm and central cardiovascular function (Gray, W., Molecular and Cellular Endocrinology 288, 52-62, 2008). Neurotensin (NT), is a 30 amino acid peptide. In the brain, NT is expressed in neurons where it acts as a neuromodulator. The effects of centrally administered NT include the interaction of the peptide with dopaminergic (DA) systems, the ability to induce opioid-independent analgesia, inhibition of food intake, and modulation of pituitary hormone release. In the periphery, NT is primarily produced throughout the mucosa and regulates a number of digestive processes. Other organs that produce NT include the heart and adrenals (Sarret and Kitabgi, Encyclopedia of Neuroscience, 1021-1034, 2009; Pons, J., et al., Current Opinion in Investigational Drugs 5, 957-962, 2004).

In the development of a potential therapeutic agent, because neprilysin cleaves a multitude of peptide substrates, many if not all of which play important physiological roles, it would be desirable to identify neprilysin variants that have enhanced specificity for cleavage of one of the substrate peptides, such as Aβ, relative to cleavage of the other (off-target) peptide substrates.

Mutants of neprilysin with a changed specificity profile have been described. Namely, mutating arginine 102 to glutamine (R102Q) leads to a differential catalytic efficiency with respect to the carboxypeptidase activity of neprilysin (Beaumont et al. (1992) J. Biol. Chem. 267:2138-41; Kim et al. (1992) J. Biol. Chem. 267:12330-35; Banos et al. (2007) Biol. Chem. 388:447-455). R747 has been found to influence selectivity as well (Beaumont et al. (1991) J. Biol. Chem. 266:214-220). Positions F563, F564, M579, F716 and 1718 have been described to influence kcat/Km for the hydrolysis of an enkephalin derivative (Marie-Claire et al (2000) Proteins 39:365-371). Positions R102 and N542 were also found to influence inhibition by small compounds (Dion et al. (1997) FEBS Lett. 411:140:144).

WO 2007/040437 describes fusion proteins of the form A-L-M, in which “A” is a protease capable of cleaving amyloid beta peptide, “L” is a linker and “M” is a component that modulates in-vivo half-life, such as the Fc part of an antibody; “A” may be human neprilysin.

WO 2008/118093 describes a fusion protein that cleaves amyloid beta peptide wherein a half-life modulating moiety is attached to the N-terminal end of human neprilysin, and a method to reduce Aβ peptide concentrations by administration of such a fusion protein as a medical therapy.

WO 2005/123119 provides a method of making a recombinant truncated mammalian neprilysin and the method of treating inflammatory bowel disease in mammals with a pharmaceutical composition comprising such truncated protein.

US2003/0083277 and US2003/0165481 describe a method of preventing formation of growth of amyloid fibrils by administration of effective amounts of an inactivating enzyme, e.g. neprilysin. Treatment can be either by administration of purified protein or viral or plasmid vector. Administration is made to the brain. US2003/0083277 describes insulin degrading enzyme for the same application.

It would be desirable to produce neprilysin variants with altered substrate specificity, in particular, variants with increased specificity for amyloid beta (Aβ) peptides, by identification of amino acid positions where mutations influence the substrate specificity of neprilysin as demonstrated herein for neprilysin variants with increased specificity for Aβ, bradykinin or neurotensin.

### SUMMARY OF THE INVENTION

The present invention provides variant neprilysin polypeptides, preferably variant human neprilysin polypeptides with improved properties. In particular, compared to wild type neprilysin, the variant neprilysin polypeptides of the invention have increased specificity for one of the neprilysin substrate peptides relative to other neprilysin substrate peptides. In particular, the present invention provides mutant/variant forms of neprilysin that, compared to wild type neprilysin, have an enhanced specificity for cleavage of Aβ than other substrates of wild type neprilysin. Such molecules, when administered as a therapeutic, may have a similar or an enhanced effect at degrading Aβ than wild type neprilysin, but a reduced effect at degrading the other neprilysin ligand substrates, compared to wild type neprilysin, thus minimising or reducing any unwanted or disadvantageous or toxic effects that might arise through degradation of these other substrates.

With respect to a variant with enhanced specificity for Aβ peptide, such variant may be useful in the treatment of Alzheimer's disease and other diseases mediated by Aβ accumulation, due to excessive Aβ formation or decreased Aβ degradation.

The present invention also relates to methods of preventing amyloid plaque formation and/or growth by reacting amyloid peptides with a composition comprising a variant neprilysin polypeptide with increased specificity for Aβ peptide so as to inactivate them through degradation or modification. The present invention in further relates to a method of treating Alzheimer's disease by administering an optimized variant neprilysin polypeptide with increased specificity and/or catalytic activity and/or selectivity and/or prolonged activity for Aβ peptide in blood plasma. The present invention also relates to the field of medical therapy, in particular to the field of neurodegenerative disease and provides methods of eliciting clearance mechanisms for brain amyloid in patients suffering from neurodegenerative diseases, in particular Alzheimer's disease. Furthermore, this invention relates to the use of proteins and peptides effective in eliciting such mechanisms.

The present invention is also directed to using a recombinant protein to treat Alzheimer's patients. In particular, to the use of a neprilysin polypeptide of the invention or a fusion protein comprising a neprilysin variant polypeptide of the invention. Compared to wild type neprilysin, the neprilysin variants of the invention possess increased specificity for binding and/or cleavage of the Aβ-peptides than binding and/or cleavage of other neprilysin substrates. It is perceived that reducing the specificity for these other substrates will minimise any off-target effects (toxic) that might arise upon administration of a neprilysin variant of the invention to a patient.

The present invention provides a polypeptide comprising a variant human neprilysin extracellular domain or a fragment thereof, said variant or fragment thereof having an amino acid sequence that differs from the wild-type human neprilysin extracellular domain shown in SEQ ID NO: 2 by at least one amino acid, wherein the polypeptide is capable of digesting an amyloid beta polypeptide with a higher specificity than wild-type neprilysin. The amyloid beta polypeptide can be human Amyloid β₁₋₄₀, and/or human Amyloid β₁₋₄₂. In the variant human neprilysin extracellular domain the amino acid G399 and/or G714 may be replaced by another naturally occurring amino acid, said naturally occurring amino acid may be an amino acid other than Ala; G399 may be replaced by Valine (V) and/or G714 may be replaced by Lysine (K); the amino acid residue numbering is based on the wild type human neprilysin sequence shown in SEQ ID NO: 1. A polypeptide according to the invention may comprise a protease variant human neprilysin extracellular domain or fragment thereof, that differs by at least one of the amino acids at positions selected from: T99, 5100, 5101, G104, D107, G195, T206, H211, H214, H217, D219, Q220, G224, 5227, R228, D229, F247, A287, 8292, L323, Y346, M376, D377, L378, 5380, 5381, F393, R394, A396, G399, E403, T404, A405, Y413, N415, G416, N417, E419, V422, A468, 1485, 1510, L514, F516, 5517, Q518, Q521, L522, K524, E533, W534, 5536, G537, V540, Y545, 5546, 5547, G548, D590, D591, N592, G593, F596, G600, W606, Q624, A649, V692, W693, Y697, Y701, N704, 5705, T708, D709, V710, 5712, G714, R735 and K745 such that the amino acid residue present in the wild-type human neprilysin extracellular domain sequence has been replaced by another naturally-occurring amino acid and wherein the amino acid residue numbering is based on the wild-type human neprilysin sequence shown in SEQ ID NO: 1. The variant protease human neprilysin extracellular domain or fragment thereof may differ from wild type human neprilysin at one or more positions selected from:

T99 by D,

S100 by I,

S101 by L, V, Y, or I,

G104 by L, M, R, V or W,

D107 by N, V or W,

G195 by V,

T206 by R,

H211 by N,

H214 by N,

H217 by N,

D219 by A,

Q220 by K,

G224 by W,

5227 by L or R

8228 by G,

D229 by N,

F247 by C or L,

A287 by S,

R292 by M,

L323 by F,

Y346 by W,

M376 by Y,

D377 by F, H, T, Y or G,

L378 by E, K or R,

S380 by K or R,

5381 by R,

F393 by S,

R394 by C, E, G, M or P,

A396 by D,

G399 by V,

E403 by H, L or S

T404 by D or F

A405 by T,

Y413 by D,

N415 by A,

G416 by R or W

N417 by W,

E419 by L, M, F or K,

V422 by M,

A468 by S,

1485 by V,

1510 by D, E, F or R,

L514 by K or F,

F516 by R,

5517 by D, F, R, W or Y,

Q518 by R or P,

Q521 by R or E,

L522 by Y,

K524 by R,

E533 by F, A or R,

W534 by C,

S536 by G, P, R, E, E or W,

G537 by E or T,

V540 by C, E, F or G,

Y545 by S or V,

S546 by D, E, I, R, W or Y,

S547 by D, E, F, G or K,

G548 by C, E, R or W,

D590 by F, M or W,

D591 by E or L,

N592 by P,

G593 by V or D,

F596 by P,

G600 by D, V or W,

W606 by S,

Q624 by H,

A649 by G,

V692 by M,

W693 by C, F, N, Q, V or L,

Y697 by G,

Y701 by G or R,

N₇O₄ by E, G, R or W,

S705 by R,

T708 by K,

D709 by K or V

V710 by F,

S712 by H, L, Q or G,

G714 by H or K,

R735 by H and

K745 by N.

A polypeptide in accordance with this aspect of the invention may comprise a moiety capable of extending half-life of the polypeptide in plasma, such as are described herein, the moiety capable of extending half-life of the polypeptide in plasma can be a human serum albumin, an Fc domain, or a fragment thereof, provided N-terminal to the variant human neprilysin extracellular domain or fragment thereof. The human serum albumin can be a variant HSA, such as the variant HSA C34S in which a cysteine residue has been replaced by a serine. The moiety capable of extending half-life of the polypeptide and protease variant human neprilysin extracellular domain or fragment thereof can, optionally, be connected via a linker. The linker can be a peptide linker, for example a glycine-serine linker such as the peptide GGGGS or GGGGGS. The present invention further provides a polypeptide comprising ^{N}HSA C34S, a GGGGS linker and a G399V/G714K variant human neprilysin extracellular domain^{C}, such as is shown in SEQ ID NO: 28

A polypeptide according to the invention suitably is capable of digesting one or more peptide selected from Angiotensin-1 and -2, ANP, BNP, bradykinin, Endothelin-1 and -2, Neuropeptide Y, Neurotensin, Adrenomedullin, Bombesin, BLP, CGRP, Enkephalins, FGF-2, fMLP, GRP, Neurokinin A, Neuromedin C, Oxytocin, PAMP, Substance P and VIP with a lower specificity than wild-type human neprilysin. The present invention also provides a nucleic acid encoding a polypeptide described above, a vector comprising said nucleic acid and a host cell comprising said vector. Additionally the invention provides method for producing a polypeptide as described above, comprising a protease variant, wherein the method comprises the following steps: (a) culturing the host cell as described above under conditions suitable for the expression of the polypeptide comprising a variant human neprilysin extracellular domain or a fragment thereof; and (b) recovering the polypeptide from the host cell culture. The present invention yet further provides pharmaceutical composition comprising a polypeptide comprising a variant human neprilysin extracellular domain or a fragment thereof in accordance with the invention and a pharmaceutically acceptable excipient. The invention also provides a polypeptide comprising a variant human neprilysin extracellular domain or a fragment thereof in accordance with the invention for use in treating a disease associated with accululation of Aβ, such as Alzheimer's disease. Also provided is a method for treating a disease associated with accumulation of Aβ, such as Alzheimer's disease, comprising administering to a patient in need thereof a therapeutically effective dose of a polypeptide comprising a variant human neprilysin extracellular domain or a fragment thereof in accordance with the invention.

### Detailed Description of Key Sequences

SEQ ID NO:1 shows the amino acid sequence of wild type human neprilysin without the codon triplet for initial methionine (Wt-full length neprilysin). The first amino acid (Y) of the human soluble Neprilysin sequence occurs at position 51.

SEQ ID NO:2 shows the amino acid sequence of wild type soluble human neprilysin (Wt-sNeprilysin;), i.e., the extracellular catalytic domain.

SEQ ID NO:3 shows the amino acid sequence of soluble human neprilysin with amino terminal 3×HA-tag and dipeptide-linker. The first amino acid (Y) of the human soluble Neprilysin sequence occurs at position 30.

SEQ ID NO:4 shows the nucleotide-sequence of wild type soluble human neprilysin (Wt-sNeprilysin).

SEQ ID NO:5 shows the nucleotide-sequence of soluble human neprilysin with amino terminal 3×HA-tag and dipeptide linker. The first codon triplet of the human soluble Neprilysin sequence (TAC) occurs at positions 88-90.

SEQ ID NO: 6 shows the nucleotide-sequence of full-length wild type human Neprilysin without the codon triplet for initial methionine.

SEQ ID NO:7 shows the nucleotide sequence of human soluble neprilysin sequence N-terminal fused to sequences encoding a secretion leader, secretion site, triple HA-tag and a dipeptide linker in expression vector pYES2. The alpha secretion leader sequence including the secretion site is at position 507-773, the 3×HA tag sequence is at position 774-854; the Gly/Ser linker (Dipeptide-linker) is at position 855-860; the sNeprilysin sequence is at position 861-2960; and the CYY1 terminator sequence is at position 3090-3338.

SEQ ID NO: 28 shows a human variant neprilysin extracellular domain that has two amino acid changes from wild-type human neprilysin: Glycine 399 to Valine and Glycine 714 to Lysine; this variant has enhanced stability and specificity:

YDDGICKSSDCIKSAARLIQNMDATTEPCTDFFKYACGGWLKRNVIPETSSRYGNFDI
LRDELEVVLKDVLQEPKTEDIVAVQKAKALYRSCINESAIDSRGGEPLLKLLPDIYGW
PVATENWEQKYGASWTAEKAIAQLNSKYGKKVLINLFVGTDDKNSVNHVIHIDQPR
LGLPSRDYYECTGIYKEACTAYVDFMISVARLIRQEERLPIDENQLALEMNKVMELEK
EIANATAKPEDRNDPMLLYNKMTLAQIQNNFSLEINGKPFSWLNFTNEIMSTVNISITN
EEDVVVYAPEYLTKLKPILTKYSARDLQNLMSWRFIMDLVSSLSRTYKESRNAFRKA
LYVTTSETATWRRCANYVNGNMMNAVGRLYVEAAFAGESKHVVEDLIAQIREVFIQ
TLDDLTWMDAETKKRAEEKALAIKERIGYPDDIVSNDNKLNNEYLELNYKEDEYFEN
IIQNLKFSQSKQLKKLREKVDKDEWISGAAVVNAFYSSGRNQIVFPAGILQPPFFSAQQ
SNSLNYGGIGMVIGHEITHGFFDNGRNPNKDDDLVDWWTQQSASNFKEQSQCMVYQ
YGNFSWDLAGGQHLNGINTLGENIADNGGLGQAYRAYQNYIKKNGEEKLLPGLDLN
HKQLFFLNFAQVWCGTYRPEYAVNSIKTDVHSPKNFRIIGTLQNSAEFSEAFHCRKNS
YMNPEKKCRVW

SEQ ID NO: 29 shows (N terminus to C-terminus) HSA (C34S variant)—GGGGS linker—human neprilysin variant with two amino acid changes from wild type neprilysin: G399V and G714K.

DAHKSEVAHRFKDLGEENFKALVLIAFAQYLQQSPFEDHVKLVNEVTEFAKTCVADE
SAENCDKSLHTLFGDKLCTVATLRETYGEMADCCAKQEPERNECFLQHKDDNPNLP
RLVRPEVDVMCTAFHDNEETFLKKYLYEIARRHPYFYAPELLFFAKRYKAAFTECCQ
AADKAACLLPKLDELRDEGKASSAKQRLKCASLQKFGERAFKAWAVARLSQRFPKA
EFAEVSKLVTDLTKVHTECCHGDLLECADDRADLAKYICENQDSISSKLKECCEKPLL
EKSHCIAEVENDEMPADLPSLAADFVESKDVCKNYAEAKDVFLGMFLYEYARRHPD
YSVVLLLRLAKTYETTLEKCCAAADPHECYAKVFDEFKPLVEEPQNLIKQNCELFEQ
LGEYKFQNALLVRYTKKVPQVSTPTLVEVSRNLGKVGSKCCKHPEAKRMPCAEDYL
SVVLNQLCVLHEKTPVSDRVTKCCTESLVNRRPCFSALEVDETYVPKEFNAETFTFHA
DICTLSEKERQIKKQTALVELVKHKPKATKEQLKAVMDDFAAFVEKCCKADDKETC
FAEEGKKLVAASQAALGLGGGGSYDDGICKSSDCIKSAARLIQNMDATTEPCTDFFK
YACGGWLKRNVIPETSSRYGNFDILRDELEVVLKDVLQEPKTEDIVAVQKAKALYRS
CINESAIDSRGGEPLLKLLPDIYGWPVATENWEQKYGASWTAEKAIAQLNSKYGKKV
LINLFVGTDDKNSVNHVIHIDQPRLGLPSRDYYECTGIYKEACTAYVDFMISVARLIR
QEERLPIDENQLALEMNKVMELEKEIANATAKPEDRNDPMLLYNKMTLAQIQNNFSL
EINGKPFSWLNFTNEIMSTVNISITNEEDVVVYAPEYLTKLKPILTKYSARDLQNLMS
WRFIMDLVSSLSRTYKESRNAFRKALYVTTSETATWRRCANYVNGNMMNAVGRLY
VEAAFAGESKHVVEDLIAQIREVFIQTLDDLTWMDAETKKRAEEKALAIKERIGYPD
DIVSNDNKLNNEYLELNYKEDEYFENIIQNLKFSQSKQLKKLREKVDKDEWISGAAV
VNAFYSSGRNQIVFPAGILQPPFFSAQQSNSLNYGGIGMVIGHEITHGFFDNGRNPNKD
DDLVDWWTQQSASNFKEQSQCMVYQYGNFSWDLAGGQHLNGINTLGENIADNGGL
GQAYRAYQNYIKKNGEEKLLPGLDLNHKQLFFLNFAQVWCGTYRPEYAVNSIKTDV
HSPKNFRIIGTLQNSAEFSEAFHCRKNSYMNPEKKCRVW

SEQ ID NO: 30 shows the sequence for the human serum albumin variant HSA C34S:

DAHKSEVAHRFKDLGEENFKALVLIAFAQYLQQSPFEDHVKLVNEVTEF
AKTCVADESAENCDKSLHTLFGDKLCTVATLRETYGEMADCCAKQEPER
NECFLQHKDDNPNLPRLVRPEVDVMCTAFHDNEETFLKKYLYEIARRHP
YFYAPELLFFAKRYKAAFTECCQAADKAACLLPKLDELRDEGKASSAKQ
RLKCASLQKFGERAFKAWAVARLSQRFPKAEFAEVSKLVTDLTKVHTEC
CHGDLLECADDRADLAKYICENQDSISSKLKECCEKPLLEKSHCIAEVE
NDEMPADLPSLAADFVESKDVCKNYAEAKDVFLGMFLYEYARRHPDYSV
VLLLRLAKTYETTLEKCCAAADPHECYAKVFDEFKPLVEEPQNLIKQNC
ELFEQLGEYKFQNALLVRYTKKVPQVSTPTLVEVSRNLGKVGSKCCKHP
EAKRMPCAEDYLSVVLNQLCVLHEKTPVSDRVTKCCTESLVNRRPCFSA
LEVDETYVPKEFNAETFTFHADICTLSEKERQIKKQTALVELVKHKPKA
TKEQLKAVMDDFAAFVEKCCKADDKETCFAEEGKKLVAASQAALGL

### DETAILED DESCRIPTION OF THE FIGURES

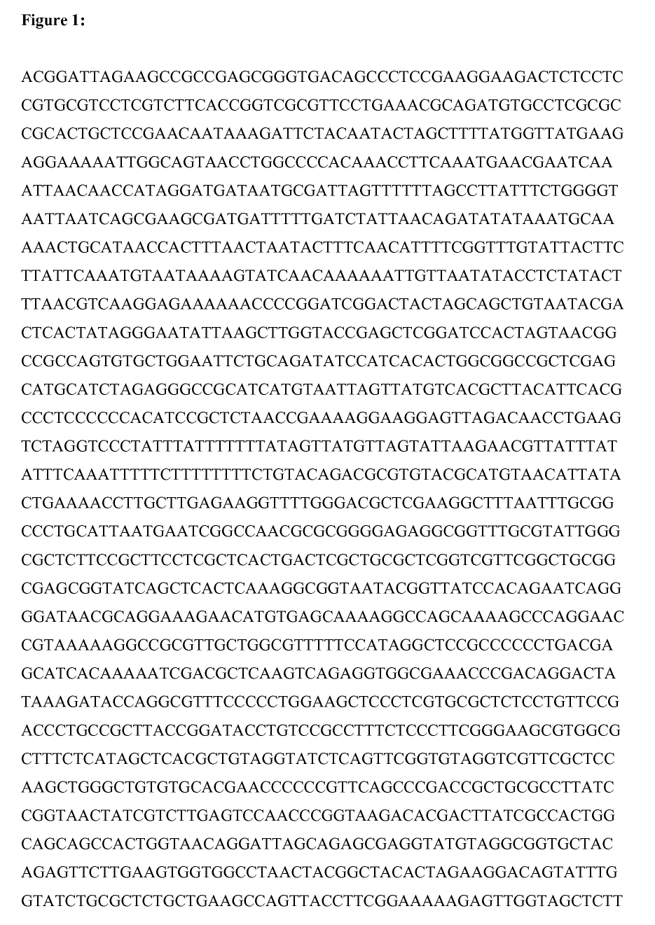

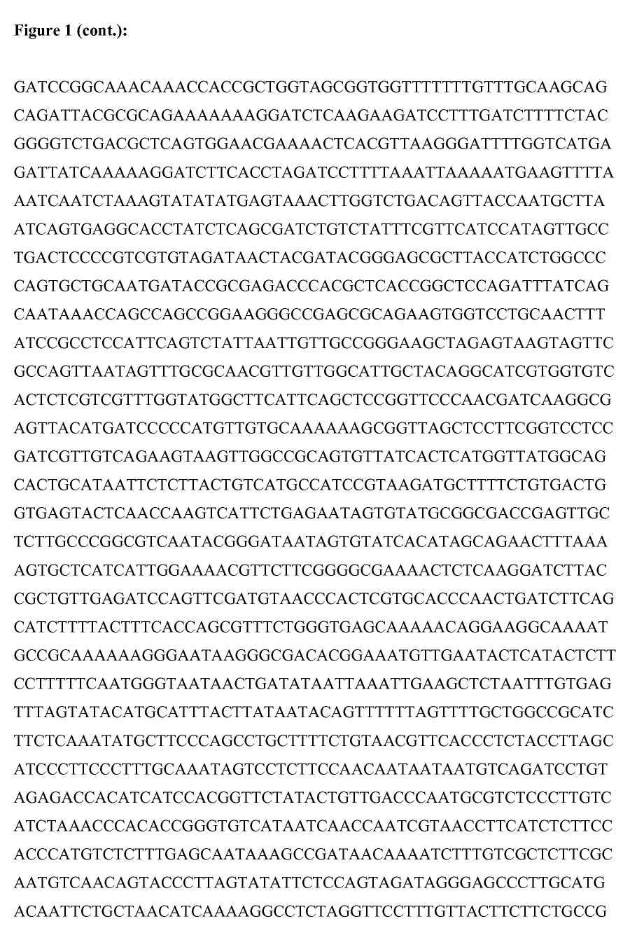

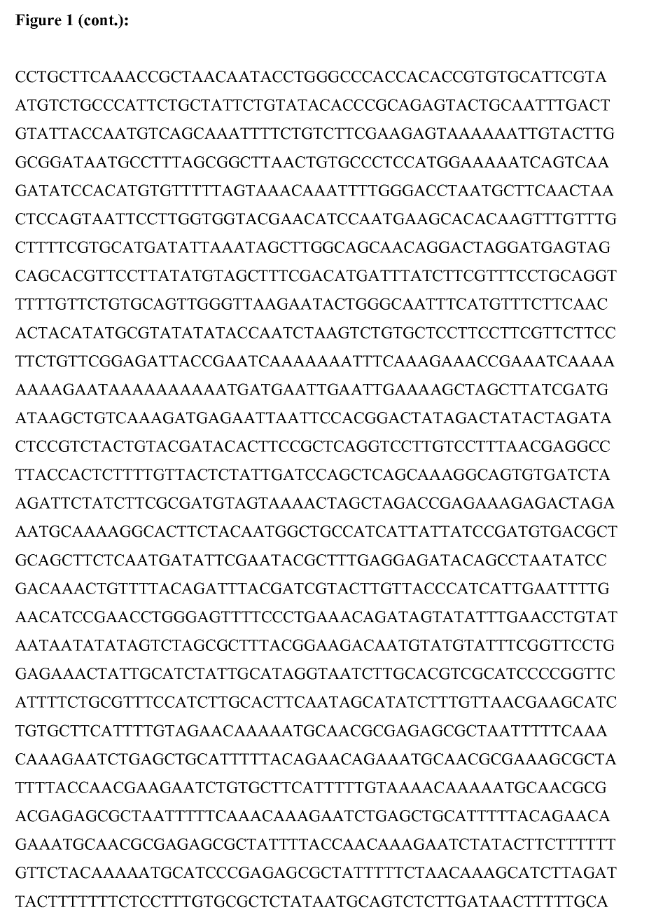

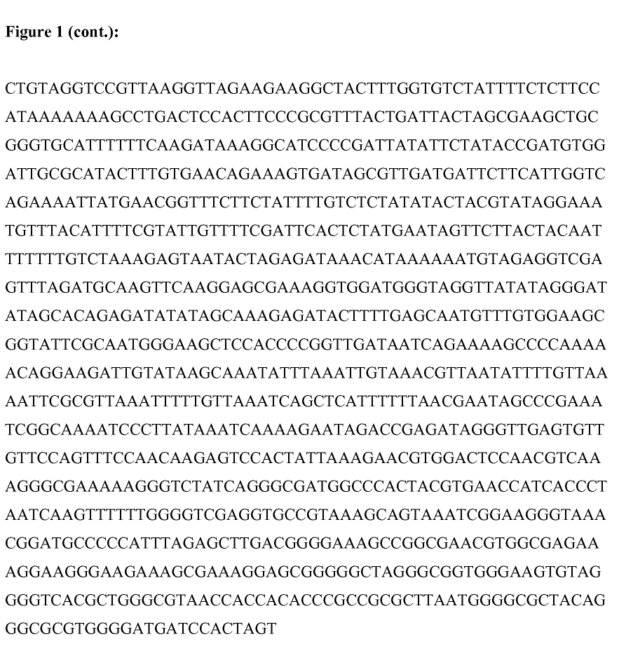

FIG. 1 shows the nucleotide sequence of yeast expression vector pYES2 (Invitrogen, SKU#V825-20), 5856 bp (SEQ ID NO: 22). The pYES2 vector is designed for native expression of your protein of interest in S. cerevisiae. It contains the URA3 gene for selection in yeast and 2μ origin for high-copy maintenance.

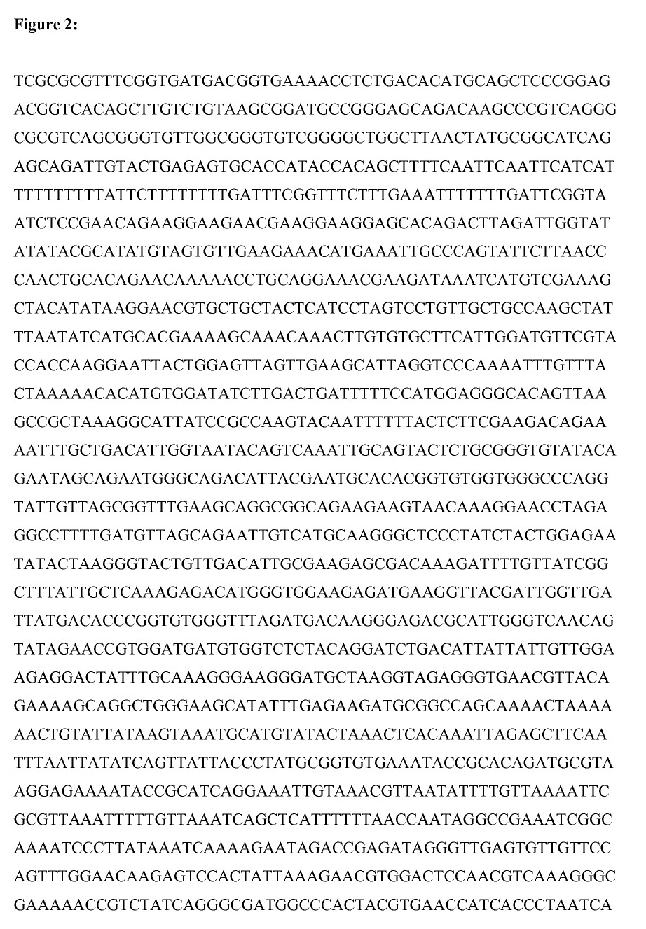

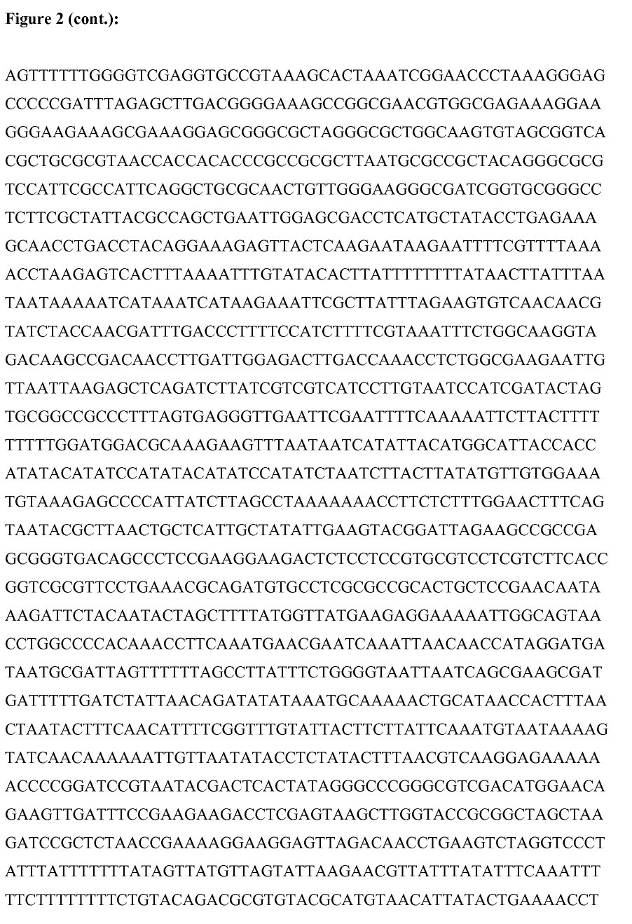

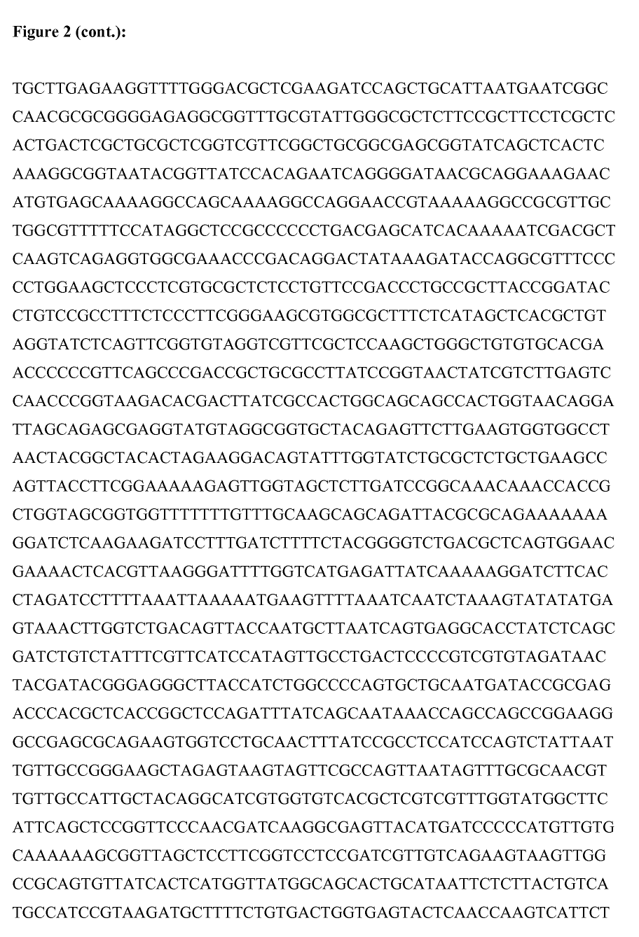

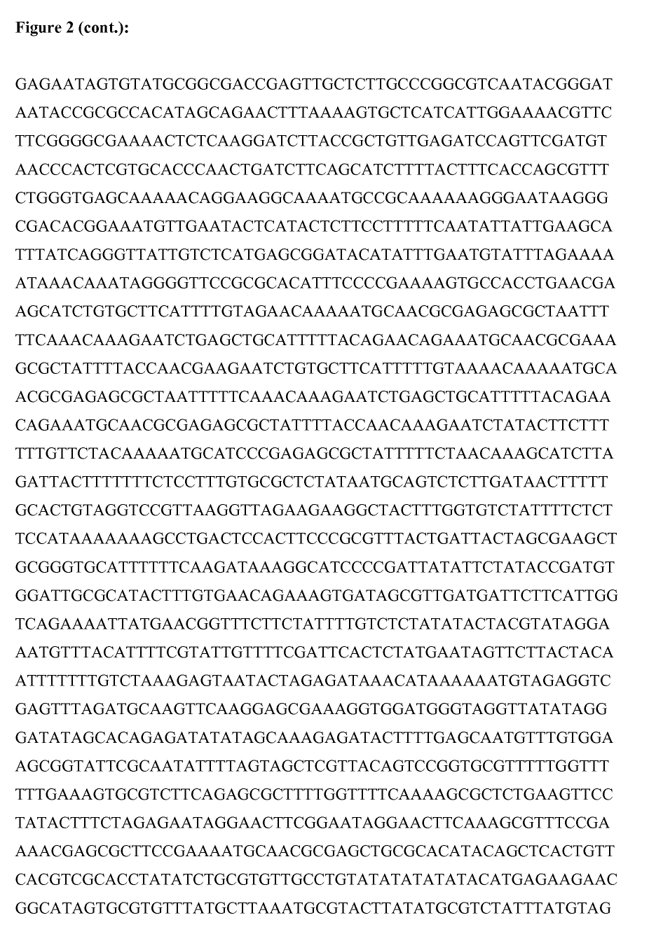

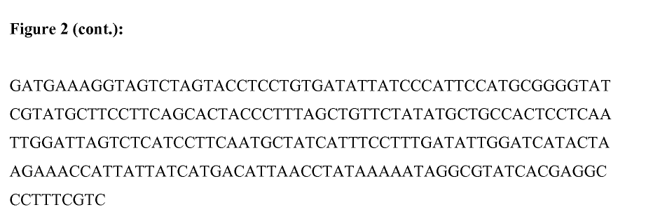

FIG. 2 shows nucleotide sequences of yeast expression vector pESC-URA (Stratagen), 6631 bp (SEQ ID NO:23).

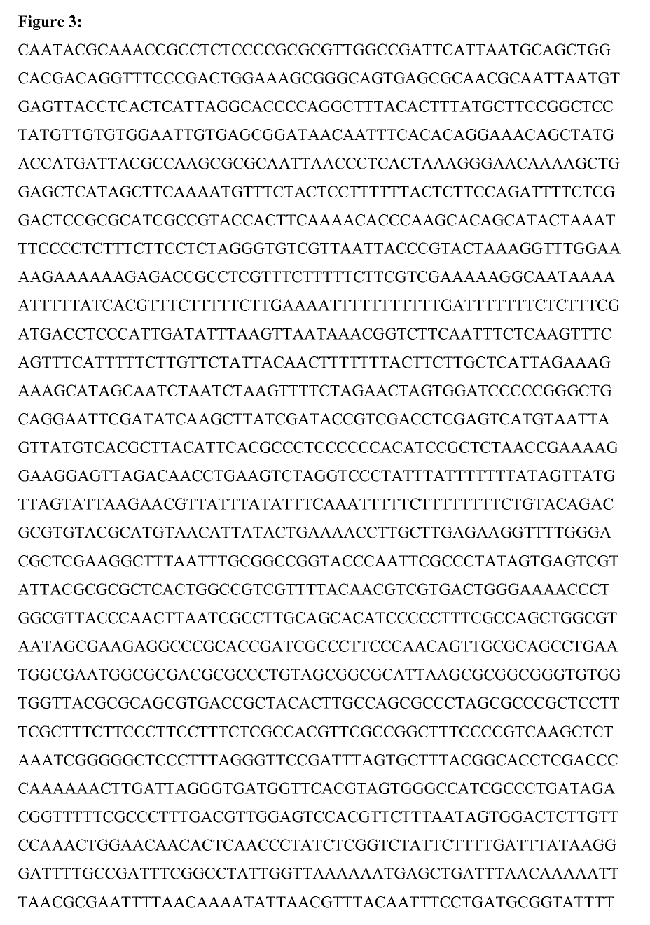

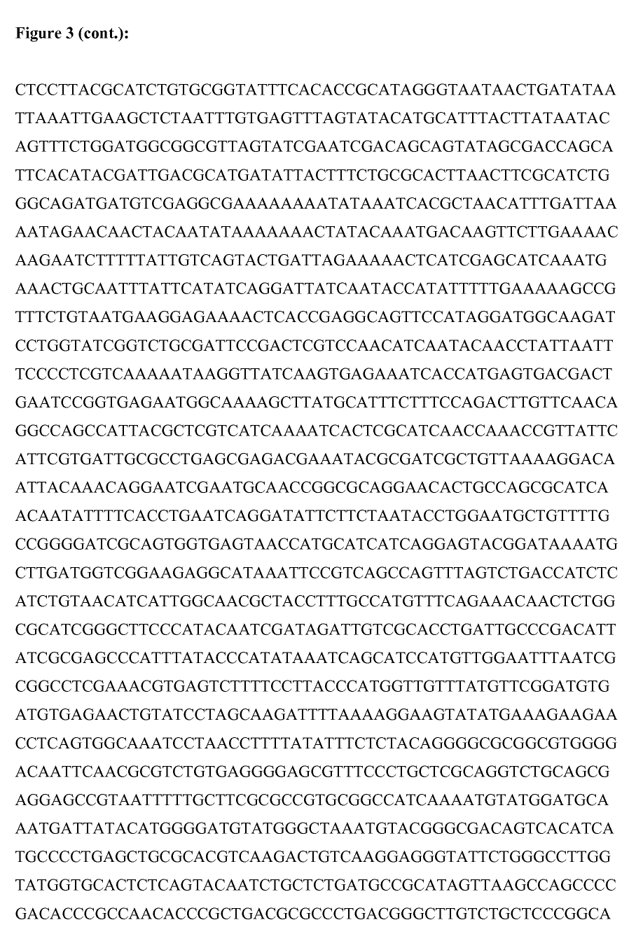

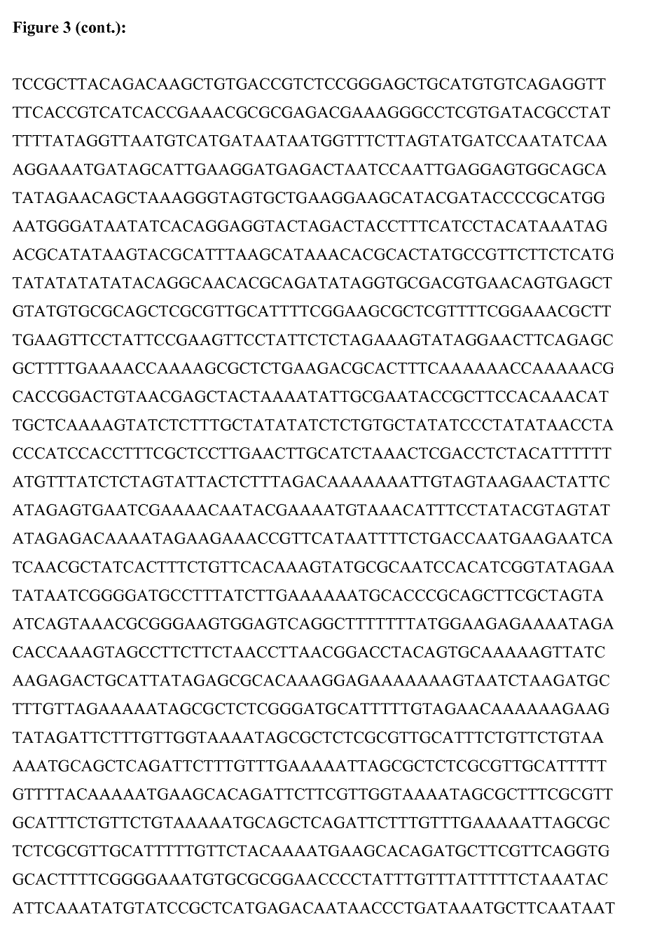

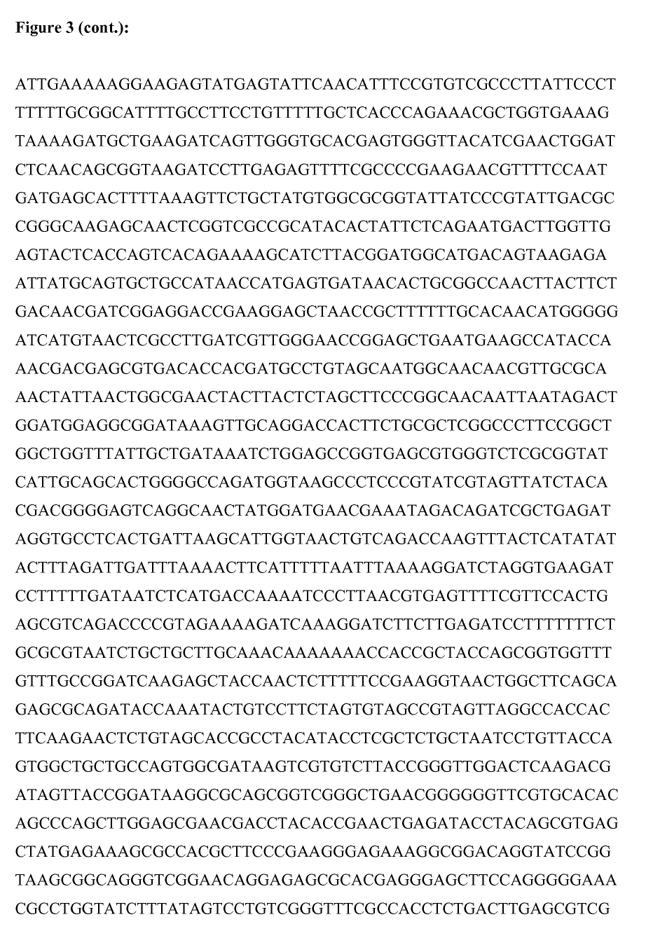

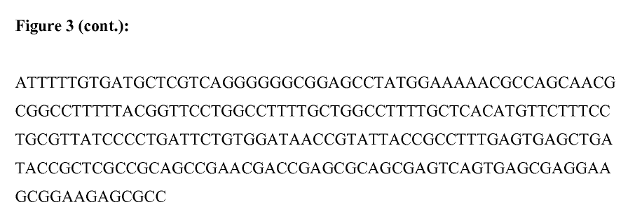

FIG. 3 shows nucleotide sequence of expression vector p427-TEF (Dualsystems Biotech), 6702 bp (SEQ ID NO:24).

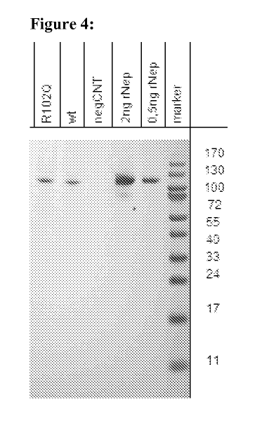

FIG. 4 shows a Western blot analysis of a culture supernatant of cells expressing human sNeprilysin (detection antibody: goat-polyclonal anti-h neprilysin (R&D)).

FIG. 5 shows the cleavage of five of the peptide substrates (peptide 5=angiotensin; peptide 3=ANP; peptide 6a=one of the endothelin peptides; peptide 1=AB₁₋₄₀; and, peptide 2=AB₁₋₄₂) by various mutants relative to the G399V/G714K parent mutant (see Table 8), illustrating the increased cleavage of the amyloid beta peptides (AB₁₋₄₀ and AB₁₋₄₂) and reduced cleavage of the three off-peptides (ANP, endothelin and angiotensin).

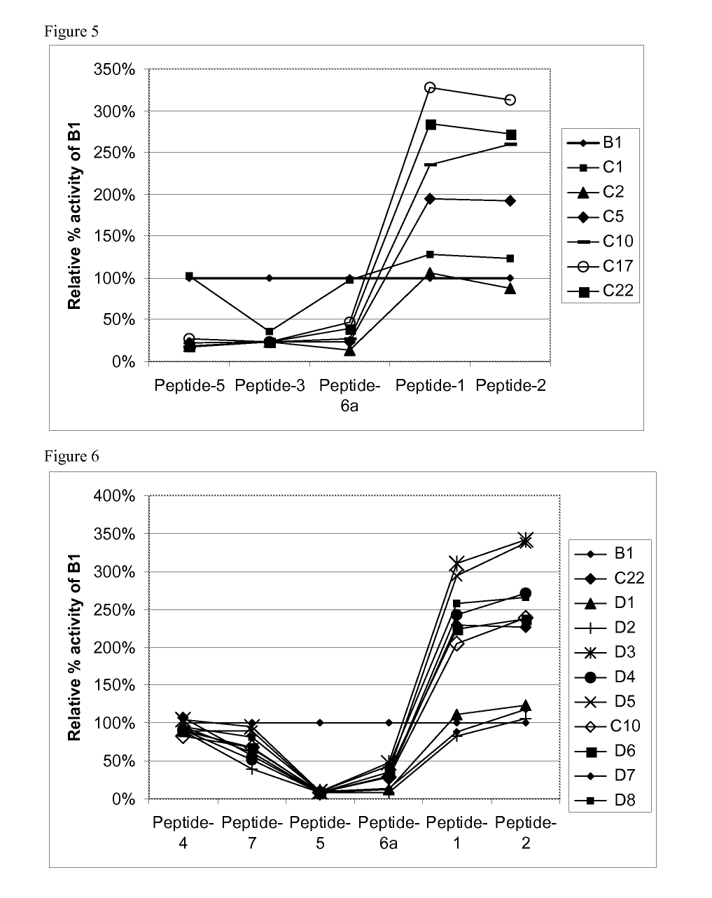

FIG. 6 shows the cleavage of six of the peptide substrates (peptide 5=angiotensin; peptide 4=BNP; peptide 7=neuropeptide Y; peptide 6a=one of the endothelin peptides; peptide 1=AB₁₋₄₀; and, peptide 2=AB₁₋₄₂) by various mutants from Table 10 relative to the G399V/G714K parent mutant.

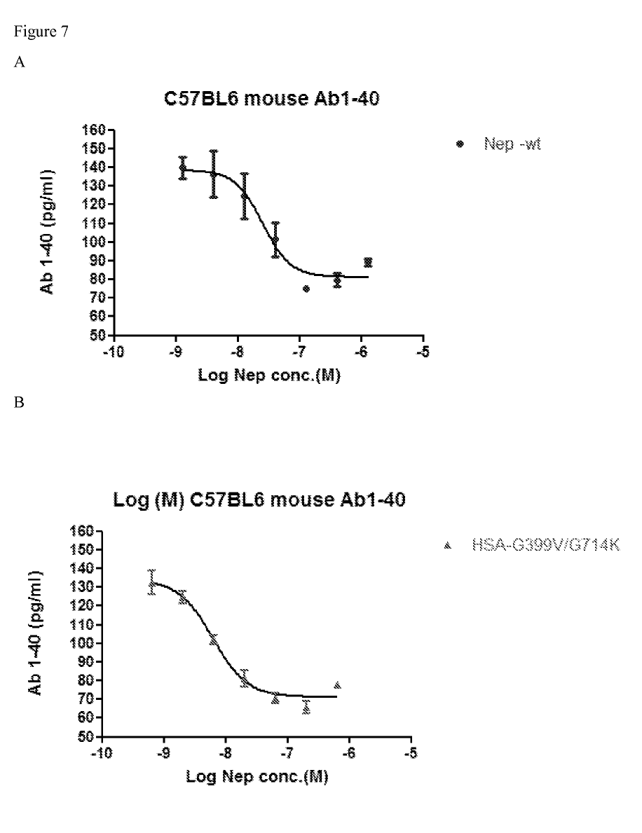

FIG. 7: Abeta degradation of edogenous mouse Abeta 1-40 in plasma from C57BL/6 mice after 1 hour incubation at RT° C. using 1 uM to 0.1 nM of enzyme. A) wildtype neprilysin. B) neprilysin variant G399V/G714K fused to HAS (N-HSA-hNepG399V/G714K-C in this and the following examples).

FIG. 8: Abeta degradation of human Abeta 1-42 in plasma from TG2576 mice after 1 hour incubation at RT° C. using 1 uM to 1 nM of enzyme. A) wildtype neprilysin. B) neprilysin variant G399V/G714K fused to HSA.

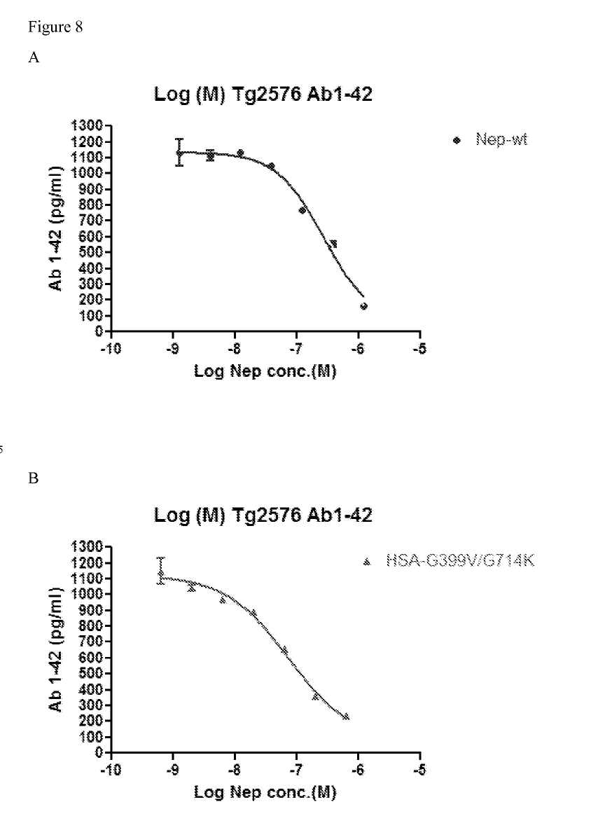

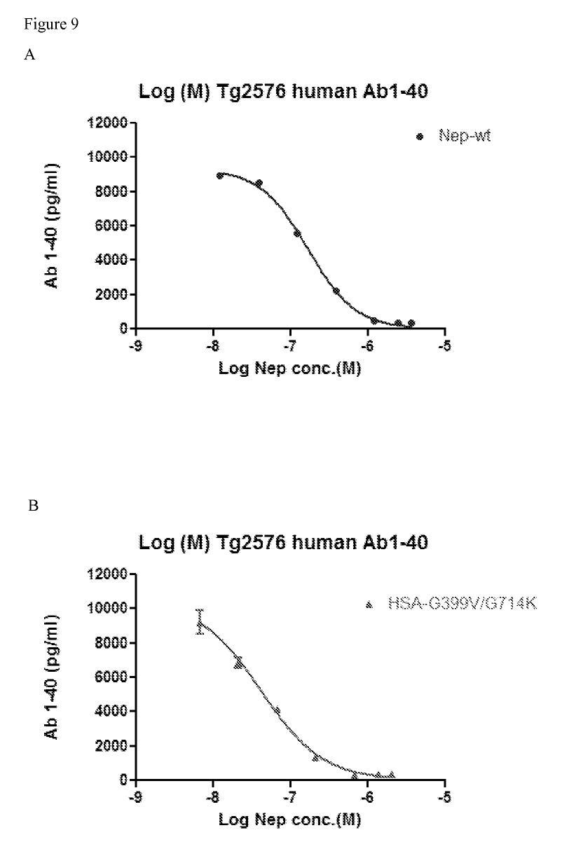

FIG. 9: Abeta degradation of human Abeta 1-40 in plasma from TG2576 mice after 1 hour incubation at RT ° C. using 3 uM to 10 nM of enzyme. A) wildtype neprilysin. B) neprilysin variant G399V/G714K fused to HSA.

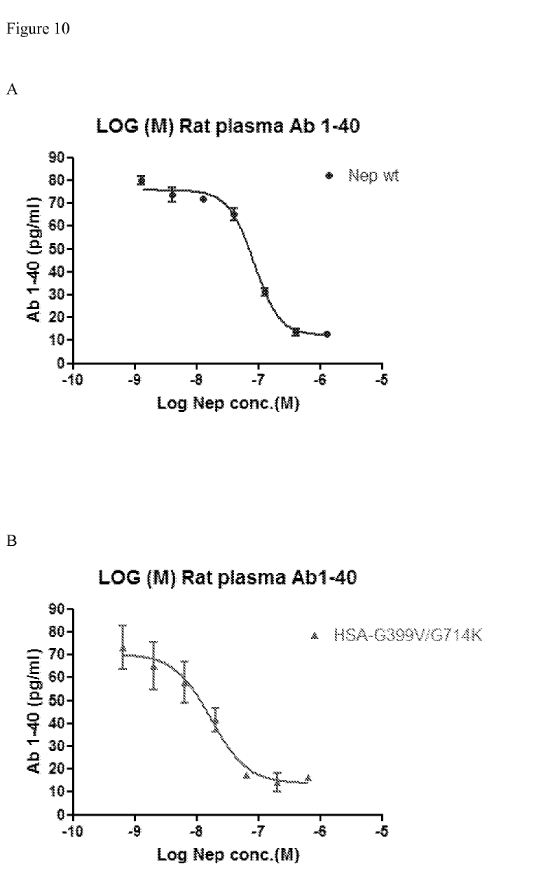

FIG. 10: Abeta degradation of rat Abeta 1-40 in plasma from Sprague Dawley rats after 1 hour incubation at RT° C. using 1 uM to 0.1 nM of enzyme. A) wildtype neprilysin. B) neprilysin variant G399V/G714K fused to HSA.

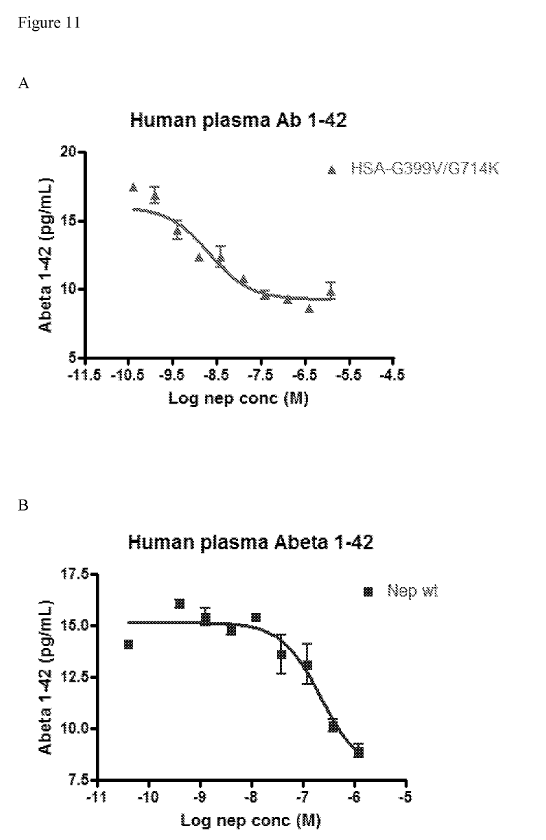

FIG. 11: Abeta degradation of Abeta 1-42 in human plasma after 1 hour incubation at RT ° C. using 3 uM to 0.1 nM of enzyme. A) wildtype neprilysin. B) neprilysin variant G399V/G714K fused to HSA.

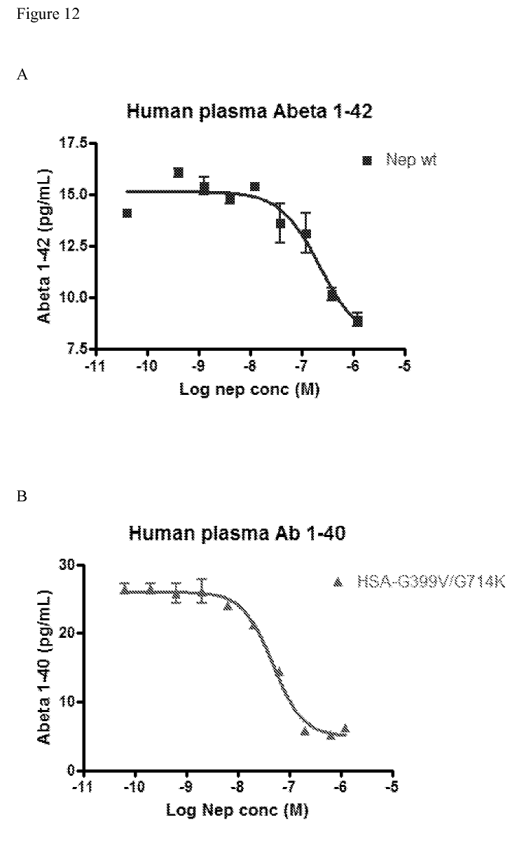

FIG. 12: Abeta degradation of Abeta 1-40 in human plasma after 1 hour incubation at RT ° C. using 1 uM to 0.1 nM of enzyme. A) wildtype neprilysin. B) neprilysin variant G399V/G714K fused to HSA.

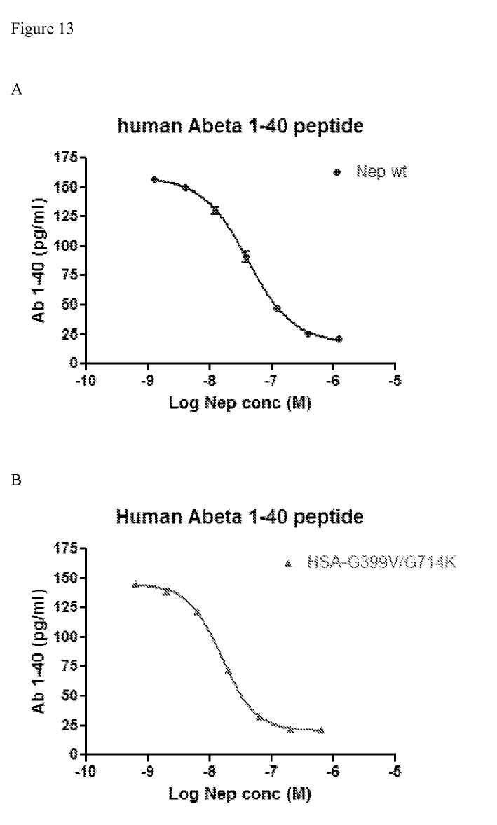

FIG. 13: Abeta degradation of Abeta 1-40 in buffer after 1 hour incubation at RT° C. using 1 uM to 1 nM of enzyme. A) wildtype neprilysin. B) neprilysin variant G399V/G714K fused to HSA.

### DETAILED DESCRIPTION OF THE INVENTION

In the framework of this invention the following abbreviations, terms and definitions are used:

aa amino acid
HA-tag Haemagglutinin epitope tag
3×HA tag 3-times the HA epitope
Nt nucleotide

### PCR Polymerase Chain Reaction

sNeprilysin soluble Neprilysin
wt wild type

The term “amyloid beta peptide”, “Aβ peptide” or “amyloid β peptide” means any form of the peptide that correlates to amino acid sequence (one letter code) DAEFRHDSG YEVHHQKLVF FAEDVGSNKG AIIGLMVGGV VIAT in the human Aβ A4 protein [Precursor], corresponding to amino acid 672 to 714 in the sequence (amino acid 1-43; Aβ1-43). It also includes any shorter forms of this peptide, such as Aβ1-40, Aβ1-41, Aβ1-42, Aβ1-39, Aβ1-38, Aβ1-43, and modified peptides such as N-terminal truncated forms as Aβ3-42, Aβ11-40 and Aβ11-42, Aβ peptides with pyroglutamyl formation as Aβ(py3-42) and Aβ(py11-42) and Aβ peptides which are modified by oxidation, isomerisation, racemization, and/or covalently linkage (ID17274, ID17231, ID17850). The term comprises also Aβ's with substitutions of residues such G1u22 for Gln (references in Soto, C. and Castano, M., (1996) Biochem. J. 314:701-707) and oligomeric forms and aggregates.

The term “polynucleotide” corresponds to any genetic material of any length and any sequence, comprising single-stranded and double-stranded DNA and RNA molecules, including regulatory elements, structural genes, groups of genes, plasmids, whole genomes, and fragments thereof.

The term “site” in a polynucleotide or polypeptide refers to a certain position or region in the sequence of the polynucleotide or polypeptide, respectively.

The term “position” in a polynucleotide or polypeptide refers to specific single bases or amino acids in the sequence of the polynucleotide or polypeptide, respectively.

The term “region” in a polynucleotide or polypeptide refers to stretches of several bases or amino acids in the sequence of the polynucleotide or polypeptide, respectively.

The term “polypeptide” comprises proteins such as enzymes, antibodies and the like, medium-length polypeptides such as peptide inhibitors, cytokines and the like, as well as short peptides down to an amino acid sequence length below ten, such as peptidic receptor ligands, peptide hormones, and the like.

The term “protease” means any protein molecule catalyzing the hydrolysis of peptide bonds. It includes naturally-occurring proteolytic enzymes, as well as protease variants and derivatives thereof. It also comprises any fragment of a proteolytic enzyme, and variants engineered by insertion, deletion, recombination and/or any other method, that leads to proteases that differ in their amino acid sequence from the naturally-occurring protease or the protease variants. It also comprises protein molecules with posttranslational and/or chemical modifications, e.g. Glycosylation, PEGylation, HESylation, gamma carboxylation and acetylation, any molecular complex or fusion protein comprising one of the aforementioned proteins.

The term “protease variant” means any protease molecule obtained by mutagenesis, preferably by site-directed or random mutagenesis with an altered amino acid sequence compared to the respective wild type sequence, which retains protease activity and may have a different substrate specificity profile when compared to the wild-type sequence.

The term “specificity” means the ability of an enzyme to recognize and convert preferentially certain substrates. The specificity of proteases, i.e. their ability to recognize and hydrolyze preferentially certain peptide substrates, can be expressed qualitatively and quantitatively. Qualitatively, proteases that digest one or a small number of peptides have a high specificity, whereas proteases that digest numerous polypeptides have a low specificity. In quantitative terms, the specificity profile of a protease is given by the respective kcat/Km ratios for all substrates, including potentially kcat/Km ratios for several cleavage sites in a given substrate.

$$\frac{{( \frac{{( \frac{{k}_{cat}}{{K}_{M}} )}_{Substrate\_i}}{{( \frac{{k}_{cat}}{{K}_{M}} )}_{Substrate\_k}} )}_{Var}}{{( \frac{{( \frac{{k}_{cat}}{{K}_{M}} )}_{Substrate\_i}}{{( \frac{{k}_{cat}}{{K}_{M}} )}_{Substrate\_k}} )}_{WT}}$$

This equation with “Var”=protease (e.g. Neprilysin) variant and “WT”=wild type (e.g. Neprilysin) protease describes the relative activities of a protease variant on “Substrate_i” and “Substrate k” in comparison to the wild-type protease. An increased specificity is expressed by ratios of 1.5, 2, 3, 4, 5, 7, 10, 20, 30, 40, 50, 100, 200 or higher. In practice, the reaction velocity kapp=(kcat/Km)* [E] ([E]=enzyme concentration) is measured. But since all measurements are done at the same enzyme concentration, the specificity as defined is independent of [E].

By enhanced specificity we mean that a variant enzymes is able to cleave amyloid beta (Aβ) peptides to a greater degree and/or other peptides (including ANP, BNP, angiotensin-1, bradykinin, endothelin 1, neuropeptide Y, neurotensin, adrenomedullin and insulin β-chain) to a lesser degree as compared to the wild-type enzyme.

By enhanced specificity for amyloid beta (Aβ), we mean that compared to wild type neprilysin, the variant neprilysin cleaves Aβ₁₋₄₀ and/or Aβ₁₋₄₂ peptide to a greater degree than any one of the following peptide substrates: ANP, BNP, angiotensin-1, bradykinin, endothelin 1, neuropeptide Y, neurotensin, adrenomedullin and insulin β-chain.

In certain embodiments it (the neprilysin variant) exhibits at least 8-fold, such as at least 10-fold, at least 20-fold, at least 30-fold, at least 50-fold, at least 60 fold, at least 70-fold, at least 80-fold, at least 90-fold and at least 100-fold, greater specificity (as measured by degree of cleavage) for Aβ than any of the other neprilysin substrate peptides. In other embodiments it exhibits at least 8-fold, such as at least 10-fold, at least 20-fold, at least 30-fold, at least 50-fold, at least 60 fold, at least 70-fold, at least 80-fold, at least 90-fold and at least 100-fold, greater specificity (as measured by degree of cleavage) for Aβ than any of: ANP, angiotensin-1, bradykinin, endothelin 1 or neurotensin. In other embodiments it exhibits at least 8-fold, such as at least 10-fold, at least 20-fold, at least 30-fold, at least 50-fold, at least 60 fold, at least 70-fold, at least 80-fold, at least 90-fold and at least 100-fold, greater specificity (as measured by degree of cleavage) for Aβ than each of: ANP, angiotensin-1, bradykinin, endothelin 1 and neurotensin. In other embodiments it exhibits at least 8-fold, such as at least 10-fold, at least 20-fold, at least 30-fold, at least 50-fold, at least 60 fold, at least 70-fold, at least 80-fold, at least 90-fold and at least 100-fold, greater specificity (as measured by degree of cleavage) for Aβ than each of: ANP, BNP, angiotensin-1, bradykinin, endothelin 1, neuropeptide Y and neurotensin.

The term “catalytic activity” describes quantitatively the conversion of a given substrate under defined reaction conditions and is proportional to kcat/Km.

The term “substrate” or “peptide substrate” comprises any peptide, oligopeptide, or protein molecule of any amino acid composition, sequence or length, and post-translational or chemically-modified forms of these molecules, that contains a peptide bond that can be hydrolyzed catalytically by a protease. The peptide bond that is hydrolyzed is referred to as the “cleavage site”.

The term “modulator” refers to a molecule that prevents degradation and/or increases plasma half-life, reduces toxicity, reduces immunogenicity, or increases biological activity of a therapeutic protein. Exemplary modulators include an Fc domain as well as a linear polymer (e.g., polyethylene glycol (PEG), polylysine, dextran, etc.); a branched-chain polymer (see, for example, U.S. Pat. No. 4,289,872, U.S. Pat. No. 5,229,490; WO 93/21259); a lipid; a cholesterol group (such as a steroid); a carbohydrate or oligosaccharide; or any natural or synthetic protein, polypeptide or peptide that binds to a salvage receptor. Glycosylation is also an example of modulator that through the increase in size of the polypeptide can prolong the plasma half-life, mainly due to a change in the clearance mechanism. A modulator can also include a human serum albumin (HSA) binding component, such as wild type human HSA or a variant human HAS, such as HSA C34S which thereby prolong the plasma half-life of the polypeptide.

The term “fusion” refers to a molecule that is composed of a modulator molecule and a protein molecule. The modulator may be covalently linked to the protein part to create the fusion protein. A non-covalent approach can also be used to connect the protein to the modulator part. The modulator part can be pegylation or glycosylation.

The term “degrade”, “degrading” or “degradation” refers to a process where one starting molecule is divided in two or more molecule(s). More specifically, the amyloid β peptide (in any size from amino acid 1-43 and smaller) is cleaved to generate smaller fragments compared to the starting molecule. The cleavage can be accomplished through hydrolysis of peptide bonds or other type of reaction, which split the molecule in smaller parts.

The term “native Fc” refers to molecule or sequence comprising the sequence of a non-antigen-binding fragment resulting from digestion of whole antibody, whether in monomeric or multimeric form. The original immunoglobulin source of the native Fc may be of human origin and may be any of the immunoglobulins, although IgG1 is preferred. Native Fcs are made up of monomeric polypeptides that may be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, IgE) or subclass (e.g., IgG1, IgG2, IgG3, IgA1, IgGA2). One example of a native Fc is a disulfide-bonded dimer resulting from papain digestion of an IgG (see Ellison et al. (1982), Nucleic Acids Res. 10: 4071-9). The term “native Fc” as used herein is generic to the monomeric, dimeric, and multimeric forms.

The term “Fc variant” refers to a molecule or sequence that is modified from a native Fc but still comprises a binding site for the salvage receptor, FcRn. Publications WO 97/34631 and WO 96/32478 describe exemplary Fc variants, as well as interaction with the salvage receptor, and are hereby incorporated by reference. Thus, the term “Fc variant” comprises a molecule or sequence that is humanized from a non-human native Fc. Furthermore, a native Fc comprises sites that may be removed because they provide structural features or biological activity that are not required for the fusion molecules of the present invention. Thus, the term “Fc variant” comprises a molecule or sequence that lacks one or more native Fc sites or residues that affect or are involved in (1) disulfide bond formation, (2) incompatibility with a selected host cell (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC). Fc variants are described in further detail hereinafter.

The term “Fc domain” encompasses native Fc and Fc variant molecules and sequences as defined above. As with Fc variants and native Fcs, the term “Fc domain” includes molecules in monomeric or multimeric form, whether digested from whole antibody or produced by other means.

The term “pharmacologically active” means that a substance so described is determined to have activity that affects a medical parameter (e.g., blood pressure, blood cell count, cholesterol level) or disease state (e.g., cancer, autoimmune disorders, dementia).

The term “half-life” is defined as the time taken for the removal of half the initial concentration of the protein or polypeptide from the plasma. This invention describes ways of modulating the half-life of neprilysin variant polypeptides in plasma. Such modification can produce fusion proteins with improved pharmacokinetic properties (e.g., increased in vivo serum half-life). Prolong the half-life means that it takes longer time for clearance of half of the initial concentration of the protein from the plasma. The half-life of a pharmaceutical or chemical compound is a well defined and well known term of the art.

The term “connect” means a covalent or a reversible linkage between two or more parts. A covalent linkage can for example be a peptide bond, disulfide bond, carbon-carbon coupling or any type of linkage that is based of a covalent linkage between to atoms. A reversible linkage can for example be biotin-streptavidin, antibody-antigen or a linkage which is classified as a reversible linkage known in the art. For example, a covalent linkage is directly obtained when the half-life modulator part and protease part of the fusion protein is produced in a recombinant form from the same plasmid, thus the connection is designed on DNA level.

The term “covalently connected” means a chemical link between two atoms in which electrons are shared between them. Examples of bonds covalently connected are a peptide bond, disulfide bond, carbon-carbon coupling. A fusion protein can be linked together by a polypeptide bond where the linkage can be accomplished during the translational process on the ribosome when the fusion protein is produced. Other type of covalently connected component could be modification with a pegylation reagent that is covalently linked to an amino residue (for example lysine) on the protein. The chemical coupling reaction can, for example, be acylation or other suitable coupling reaction which link the two components together into a fusion protein. Covalently connected can also mean a linkage of a linker at two sites in which the modulator is linked together with the protein part.

The term “cleavage sites” means a specific location/site in a peptide sequence that can be cleaved by a protein or an enzyme. Cleavage is normally produced by hydrolysis of the peptide bond connecting two amino acids. Cleavage can also take place at multiple sites in the same peptide using a single or a combination of proteins or enzymes. A cleavage site can also be other site than the peptide bond. This invention describes the cleavage of the amyloid β peptide in detail.

In some embodiments, the protease variant, or polypeptide comprising the protease variant, e.g. a fusion polypeptide, or a derivative of any of the aforesaid, or a nucleic acid encoding same is isolated. An isolated biological component (such as a nucleic acid molecule or protein such as a protease) is one that has been substantially separated or purified away from other biological components in the cell of the organism in which the component naturally occurs, e.g., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Amino acids are referred to herein using the name of the amino acid, the three-letter abbreviation or the single letter abbreviation. The table below provides a list of the standard amino acids together with their abbreviations.

| | | |
|---|---|---|
| Alanine | A | Ala |
| Cysteine | C | Cys |
| Aspartic acid | D | Asp |
| Glutamic acid | E | Glu |
| Phenylalanine | F | Phe |
| Glycine | G | Gly |
| Histidine | H | His |
| Isoleucine | I | Ile |
| Lysine | K | Lys |
| Leucine | L | Leu |
| Methionine | M | Met |
| Asparagine | N | Asn |
| Proline | P | Pro |
| Glutamine | Q | Gln |
| Arginine | R | Arg |
| Serine | S | Ser |
| Threonine | T | Thr |
| Valine | V | Val |
| Tryptophan | W | Trp |
| Tyrosine | Y | Tyr |
| Cysteine | C | Cys |

In addition to the specific amino acid variations and nucleic acids encoding the variations, conservative amino acid substitutions of the variations are provided herein. Such substitutions are those, which are conservative, for example, wherein the variant amino acid is replaced by another amino acid of the same class. Amino acids can be classified as acidic, basic, neutral and polar, or neutral and nonpolar and/or aromatic, depending on their side chain. Preferred substitutions of a variant amino acid position include those that have one or more classifications that are the same as the variant amino acid at that position. Thus, in general, amino acids Lys, Arg, and His are basic; amino acids aspartic and glutamic are acidic; amino acids Ser, Thr, Cys, Gln, and Asn are neutral polar; amino acids Gly, Ala, Val, Ile, and Leu are non-polar aliphatic, and amino acids Phe, Trp, and Tyr are aromatic. Gly and Ala are small amino acids and Val, Ile and Leu are aliphatic amino acids.

It is well known to one of ordinary skill in the art that the genetic code is degenerate, that a particular amino acid can be encoded by more than one codon triplet. Therefore, the nucleic acids provided herein also include alternate sequences that use different codons to encode the same amino acid sequence. Furthermore, the nucleic acids provided herein also include both the coding sequence and the complementary sequence of nucleic acids encoding a variant neprilysin polypeptides provided herein.

A protease variant or derivative thereof provided herein can be prepared by recombinant expression of nucleic acid sequences encoding the same in a host cell. To express a protease or derivative thereof recombinantly, a host cell can be transfected with one or more recombinant expression vectors carrying DNA fragments encoding the protease or derivative thereof such that the protease or derivative are expressed in the host cell. Standard recombinant DNA methodologies are used prepare and/or obtain nucleic acids encoding the protease or derivative thereof, incorporate these nucleic acids into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989).

To create a polynucleotide sequence that encodes a protease or derivative thereof fused to another polypeptide, protease-encoding nucleic acids can be operatively linked to another fragment encoding a flexible linker such that the protease and other polypeptide sequences can be expressed as a contiguous single-chain protein, with the protease and other polypeptide regions joined by the flexible linker.

To express the proteases or derivatives thereof standard recombinant DNA expression methods can be used (see, for example, Goeddel; Gene Expression Technology. Methods in Enzymology 185, Academic Press, San Diego, Calif (1990)). For example, DNA encoding the desired polypeptide can be inserted into an expression vector which is then transfected into a suitable host cell. It is understood that the design of the expression vector, including the selection of regulatory sequences is affected by factors such as the choice of the host cell, the level of expression of protein desired and whether expression is constitutive or inducible.

Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al. The recombinant expression vectors can also include origins of replication and selectable markers (see e.g., U.S. Pat. Nos. 4,399,216, 4,634,665 and U.S. Pat. No. 5,179,017, by Axel et al.). Suitable selectable markers include genes that confer resistance to drugs such as G418, hygromycin or methotrexate, on a host cell into which the vector has been introduced. For example, the dihydrofolate reductase (DHFR) gene confers resistance to methotrexate and the neo gene confers resistance to G418.

Transfection of the expression vector into a host cell can be carried out using standard techniques such as electroporation, calcium-phosphate precipitation, and DEAE-dextran transfection.

Suitable mammalian host cells for expressing the variant protease polypeptides provided herein include Chinese Hamster Ovary (CHO cells) (including dhfr- CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and SP2 cells. In some embodiments, the expression vector is designed such that the expressed protein is secreted into the culture medium in which the host cells are grown. The proteases or derivatives thereof can be recovered from the culture medium using standard protein purification methods.

The variant protease polypeptides can also be produced in prokaryotic cells using suitable vectors as described, for example, in U.S. Pat. No. 6,204,023 to Robinson, et al. and in (Carter et al., Bio/Technology 10:163-167 (1992). The expression vector can be designed to allow the expressed polypeptide to be secreted into the periplasmic space, or the polypeptide can be retained within the cell, for example, in inclusion bodies. The expressed polypeptide can be isolated from the periplasmic space or the inclusion bodies can be isolated from the host cell, respectively.

Suitable host cells for cloning or expressing the DNA in the vectors described herein are the prokaryote, yeast, or higher eukaryote cells described above. In addition to prokaryotes, eukaryotic microbes such as filamentous fungi or yeast are suitable cloning or expression hosts for the antibodies, antigen binding portions, or derivatives thereof provided herein. Saccharomyces cerevisiae, is a suitable eukaryotic host microorganism. Another suitable yeast host is Schizosaccharomyces pombe. Suitable host cells for the expression of a glycosylated protease or derivative thereof provided herein include mammalian, plant, and insect cells.

Host cells are transformed with the above-described expression or cloning vectors for the variant protease polypeptide and cultured in conventional nutrient media modified as appropriate for inducing promoters, selecting transformants, or amplifying the genes encoding the desired sequences. Commercially available media such as Ham's F10, Minimal Essential Medium ((MEM), RPMI-1640, and Dulbecco's Modified Eagle's Medium (DMEM), are suitable for culturing the host cells. The culture conditions, such as temperature, pH, and the like, are those previously used with the host cell selected for expression, and will be apparent to the ordinarily skilled artisan. Where the protease or derivative thereof is secreted into the medium, supernatants from such expression systems are generally first concentrated using a commercially available protein concentration filter, for example, an Amicon or Millipore Pellicon ultrafiltration unit. The protease or derivative thereof composition prepared from the cells can be purified using, for example, hydroxylapatite chromatography, gel electrophoresis, dialysis, and affinity chromatography.

In general, the protease variants described herein have pharmacological activity resulting from their ability to process/degrade pharmacological active substrates. An altered activity and/or specificity by a factor of two is sufficient to change the pharmacological activity of the variant compared to wild type. The activity/specificity of the protease variants can be determined by assays known in the art. In vivo assays are known in the art and further described in the examples section. Such pharmaceutical compositions may be for administration for injection, or for oral, pulmonary, nasal, transdermal, sub-cutaneous or other forms of administration. In general, the invention encompasses pharmaceutical compositions comprising effective amounts of a variant protease polypeptide of the invention together with pharmaceutically acceptable diluents, preservatives, solubilisers, emulsifiers, adjuvants and/or carriers. Such compositions include diluents of various buffer content, pH and ionic strength; additives such as detergents and solubilising agents, anti-oxidants, preservatives and bulking substances; incorporation of the material into particulate preparations of polymeric compounds such as polylactic acid, polyglycolic acid, etc. or into liposomes. acid may also be used, and this may have the effect of promoting sustained duration in the circulation. Such compositions may influence the physical state, stability, rate of in vivo release, and rate of in vivo clearance of the present protease variants and derivatives thereof. See, e.g. Remington's Pharmaceutical Sciences, 18th Ed. (1990, Mack Publishing Co., Easton, Pa. 18042) pages 1435-1712 which are herein incorporated by reference. The variant protease polypeptides may be prepared in liquid form, or may be in dried powder, such as lyophilized form. Implantable sustained release formulations are also contemplated, as are transdermal formulations. These administration alternatives are well known in the art.

The variant protease polypeptides provided herein can be administered to a patient in need thereof. A variety of routes can be used to administer the protease or derivative thereof. Any mode of administration that is medically acceptable, meaning any mode that produces effective levels of the active compounds without causing clinically unacceptable adverse effects can be used to administer the protease or derivative thereof. Such modes of administration include oral, sublingual, topical, nasal, transdermal or parenteral routes. The term “parenteral” includes subcutaneous, intravenous, intramuscular, or infusion.

The variant protease polypeptides can be administered once, continuously, such as by continuous pump, or at periodic intervals. The periodic interval may be weekly, bi-weekly, or monthly. The dosing can occur over the period of one month, two months, three months or more to elicit an appropriate response. Desired time intervals of multiple doses of a particular composition can be determined without undue experimentation by one skilled in the art. Other protocols for the administration of a protease or derivative thereof will be known to one of ordinary skill in the art, in which the dose amount, schedule of administration, sites of administration, mode of administration and the like vary from the foregoing.

The present invention relates to protease variant polypeptides, which are derived from human neprilysin having an altered activity and/or specificity. In a preferred embodiment the protease variants are derived from human neprilysin having an improved activity against certain proteins and peptides. In one embodiment the neprilysin variant has an improved specificity or activity against Aβ peptide. In other embodiments, the protease variants, which are derived from human neprilysin have an improved activity against certain proteins and peptides other than Aβ peptide. Examples of the certain non-Aβ peptide proteins and peptides cleavable by the protease variants are Angiotensin-1 and -2, ANP, BNP, bradykinin, Endothelin-1 and -2, Neuropeptide Y and Neurotensin, as well as Adrenomedullin, Bombesin, BLP, CGRP, Enkephalins, FGF-2, fMLP, GRP, Neurokinin A, Neuromedin C, Oxytocin, PAMP, Substance P and VIP.

Yet another embodiment is a protease variant according to any of the aforementioned variants having an altered specificity against at least one substrate selected from the group consisting of Amyloid β₄₀ Amyloid β₄₂/Angiotensin-1 and -2, ANP, BNP, bradykinin, Endothelin-1 and -2, Neuropeptide Y, Neurotensin, Adrenomedullin, Bombesin, BLP, CGRP, Enkephalins, FGF-2, fMLP, GRP, Neurokinin A, Neuromedin C, Oxytocin, PAMP, Substance P or VIP. A further embodiment is a protease variant according to any of the aforementioned variants having an altered specificity against at least one substrate selected from the group consisting of Amyloid β₄₀ Amyloid β₄₂/Angiotensin-1 and -2, ANP, BNP, bradykinin, Endothelin-1 and -2, Neuropeptide Y or Neurotensin. A further embodiment is a protease variant according to any of the aforementioned variants having an altered specificity against at least Amyloid β₄₀ or Amyloid β₄₂

The following table lists relative activities of protease variants vs. wild type neprilysin on different substrates determined from the ratio of the two corresponding kₐₚₚ-values (see example 3). These variants are representatives of the set of all protease variants defined by one mutation or a combination of mutations at position(s) named in column 1 For the purpose of exemplary illustration:

Protease variant G399V shows a 1.43-fold increased activity on Peptide-1 (Aβ peptide derivative), a 1.21-fold increased activity on Peptide-2 (Aβ peptide derivative), a 1.32-fold increased activity on Peptide-7 (NPY derivative), a 50-fold decreased activity on Peptide-8 (neurotensin derivative) and Peptide-13 (Bradykinin derivative), and a 12.5-fold decrease on Peptide-5 (angiotensin derivative). With the specificity definition above comparing a protease variant with the wild type protease this variant G399V shows an approximate 70-fold increased specificity for Peptide-1 vs. Peptide-13.

Protease variant G714K shows a 6.91-fold increased activity on Peptide-1 (Aβ peptide derivative), a 3.99-fold increased activity on Peptide-2 (Aβ peptide derivative), a 1.31-fold increased activity on Peptide-6 (endothelin derivative), and a 5-fold decreased activity on Peptide-13 (Bradykinin derivative) and Peptide-4 (BNP derivative). With the specificity definition above comparing a protease variant with the wild type protease this variant G714K shows an approximate 35-fold increased specificity for Peptide-1 vs. Peptide-13.

Protease variant G600W shows a 1.91-fold increased activity on Peptide-1 (Aβ peptide derivative), a 1.95-fold increased activity on Peptide-4 (BNP derivative), and a 100-fold decreased activity on Peptide-13 (Bradykinin derivative), With the specificity definition above comparing a protease variant with the wild type protease this variant G600W shows a nearly 200-fold increased specificity for Peptide-1 vs. Peptide-13 and a nearly 200-fold increased specificity for Peptide-4 vs. Peptide-13, however, the ratio of the activities on Peptide-1 and Peptide-4 is nearly one meaning no change in specificity regarding Peptide-1 vs. Peptide-4.

Protease variant N592P shows a 1.49-fold increased activity on Peptide-6 (Endothelin derivative), a 1.35-fold decreased activity on Peptide-8 (Neurotensin derivative), and a 2.84-fold decreased activity on Peptide-13 (Bradykinin derivative). This variant shows a 4-fold increased specificity for Peptide-6 vs. Peptide-13 and a 2-fold increased specificity for Peptide-6 vs. Peptide-8 and for Peptide-8 vs. Peptide-13.

Protease variant W693L shows a 2.15-fold increased activity on Peptide-13 (Bradykinin derivative), a 6.25-fold decreased activity on Peptide-4 (BNP derivative), and a nearly unchanged activity on Peptide-5 (Angiotensin-1 derivative). This variant shows a 13-fold increased specificity for Peptide-13 vs. Peptide-4 and a 2-fold increased specificity for Peptide-13 vs. Peptide-5 and a 6.5-fold increased specificity for Peptide-5 vs. Peptide-4.

The protease variant with a combination of the mutations W693L and G399V, however, shows a 6.7-fold decreased activity on Peptide-13 (Bradykinin derivative), shows a 6.7-fold decreased activity on Peptide-13 (Bradykinin derivative), and a 3.3-fold decreased activity on Peptide-4 (BNP derivative), resulting in a 2-fold increased specificity for Peptide-4 vs. Peptide-13.

Protease variant S536E shows a 1.37-fold increased activity on Peptide-5 (Angiotensin derivative), a 3-fold decreased activity on Peptide-13 (Bradykinin), and a 4-fold decreased activity on Peptide-1 (Aβ peptide derivative). This variant shows a 5-fold increased specificity for Peptide-5 vs. Peptide-1 and a 4-fold increased specificity for Peptide-5 vs. Peptide-13. However, a protease variant with a basic instead of a acidic residue in position 536, namely variant S536R, shows a 2.3-fold decreased activity on peptide-5 and a 3.85-fold increased activity on peptide-1, hence an inverse specificity regarding this pair of substrates.

Surprisingly mutations at position 102 resulting in residues other than the Gln (O) described in the literature showed different specificities than wt or the R102Q mutation, for example the specificity of the mutant R102P on Peptide-5 vs.-1 0.66-fold decreased, whereas R102Q and R102M show a 3- and 3.6-fold increased specificity, respectively.

Surprisingly two mutations other than I->A at pos 718 described in the literature showed different specificities, 1718L shows a 9-fold and 1718V a 2.5-fold increased specificity on Peptide-1 vs.-6.

TABLE 3

| | muta- | muta- | | | | | | | | | | |
|---|---|---|---|---|---|---|---|---|---|---|---|---|
| | tion 1 | tion 2 | Peptide-1 | Peptide-2 | Peptide-5 | Peptide-8 | Peptide-13 | Peptide-3 | Peptide-6 | Peptide-10 | Peptide-7 | Peptide-4 |
| T99 | D | | 2.42 | | 2.91 | 2.74 | 4.01 | 3.96 | 2.18 | | | |
| S100 | I | G104M | 1.40 | | 0.51 | 0.94 | 0.32 | 0.82 | 1.09 | | | |
| S101 | L | | 2.06 | 1.63 | 0.70 | 0.39 | 0.17 | 0.66 | 1.02 | 1.13 | 1.18 | 0.57 |
| S101 | V | | 1.77 | 1.40 | 0.04 | 0.02 | 0.10 | 0.13 | 0.34 | 0.52 | 0.85 | 1.55 |
| S101 | Y | | 0.90 | | 0.47 | 0.44 | 0.65 | 0.50 | 0.57 | | | |
| S101 | I | G399V | 1.76 | 1.04 | 0.05 | 0.02 | 0.01 | 0.16 | 0.38 | 0.33 | 1.03 | 0.99 |
| R102 | C | | 0.37 | 0.32 | 1.20 | 0.52 | 1.15 | 1.06 | 0.47 | | | |
| R102 | L | | 0.25 | 0.27 | 0.56 | 0.39 | 0.94 | 0.77 | 0.25 | | | |
| R102 | M | | 0.41 | | 1.47 | | | | | | | |
| R102 | P | | 0.94 | 0.91 | 0.62 | 0.33 | 0.53 | 0.69 | 0.18 | | | |
| R102 | Q | | 0.30 | 0.40 | 0.91 | 0.50 | 1.25 | 1.24 | 0.39 | | | |
| R102 | S | | | | | 0.91 | 1.93 | | | | | |
| R102 | S | S546Y | | | | 0.57 | 1.39 | | | | | |
| R102 | W | | 0.08 | 0.01 | 0.11 | 0.09 | 0.15 | 0.15 | 0.06 | | | |
| G104 | L | | 2.25 | | 0.83 | 1.60 | 0.49 | 1.40 | 1.45 | | | |
| G104 | M | S100I | 1.40 | | 0.51 | 0.94 | 0.32 | 0.82 | 1.09 | | | |
| G104 | R | | 1.90 | | 0.38 | 0.84 | 0.17 | 0.57 | 1.10 | | | |
| G104 | V | | 1.44 | | 0.83 | 1.35 | 0.58 | 1.32 | 1.37 | | | |
| G104 | W | | 0.77 | | 0.37 | 0.81 | 0.29 | 0.38 | 0.86 | | | |
| D107 | N | | 0.45 | | 0.31 | 0.19 | 0.18 | 0.18 | 0.38 | | | |
| D107 | V | | 0.50 | | 0.18 | 0.19 | 0.22 | 0.21 | 0.19 | | | |
| D107 | W | | 1.90 | 1.53 | 0.29 | 0.29 | 0.32 | 0.24 | 0.41 | 0.81 | 0.58 | 0.23 |
| G195 | V | E533R | 1.24 | | 0.50 | 0.67 | 0.53 | 0.58 | 0.61 | | | |
| T206 | R | | 1.76 | | 2.25 | 1.44 | 1.50 | 1.45 | 2.16 | | | |
| H211 | N | Y545V | 0.64 | | 0.81 | 0.70 | 0.25 | 0.41 | 0.65 | | | |
| H214 | R | | 1.93 | | 1.00 | 0.88 | 0.92 | 1.08 | 1.07 | | | |
| H217 | N | | | | | 0.92 | 1.51 | | | | | |
| D219 | A | S712G | | | | 0.35 | 1.00 | | | | | |
| Q220 | K | | 2.92 | 1.62 | 1.05 | 0.76 | 0.63 | 1.16 | 1.64 | 1.11 | 2.04 | 1.61 |
| G224 | W | D229N | 1.10 | 1.14 | 0.23 | 0.11 | 0.09 | 0.25 | 0.57 | 0.62 | 0.56 | 0.47 |
| S227 | L | | 3.28 | 2.105 | 1.51 | 0.81 | 0.37 | 1.34 | 1.67 | 1.209 | 1.406 | 1.906 |
| S227 | R | | 3.05 | 2.195 | 0.64 | 0.22 | 0.12 | 0.54 | 1.32 | 0.884 | 1.198 | 0.38 |
| R228 | V | | 1.00 | 0.93 | 0.62 | 0.81 | 0.91 | 1.03 | 0.60 | 1.03 | 0.90 | 1.04 |
| R228 | G | F247L | 6.15 | 3.37 | 0.83 | 0.65 | 0.31 | 0.66 | 1.31 | 0.78 | 0.55 | 0.96 |
| D229 | N | G224W | 1.10 | 1.14 | 0.23 | 0.11 | 0.09 | 0.25 | 0.57 | 0.62 | 0.56 | 0.47 |
| F247 | C | | 1.96 | 1.59 | 0.78 | 0.77 | 0.84 | 0.80 | 0.65 | 1.11 | 0.67 | 7.35 |
| F247 | L | R228G | 6.15 | 3.37 | 0.83 | 0.65 | 0.31 | 0.66 | 1.31 | 0.78 | 0.55 | 0.96 |
| A287 | S | D377G | 8.906 | 5.574 | 2.669 | 2.44 | 2.45 | 1.928 | 1.998 | 1.499 | 2.709 | 1.736 |
| R292 | M | | 1.416 | | 0.641 | 0.52 | 0.46 | 1.086 | 0.672 | | | |
| L323 | F | S547F | 3.00 | 2.06 | 1.35 | 1.54 | 1.45 | 1.44 | 0.88 | 1.03 | 1.14 | 0.53 |
| Y346 | W | | 1.75 | | 1.94 | 1.01 | 1.18 | 1.56 | 1.49 | | | |
| M376 | E | | 0.39 | | 0.89 | | | | | | | |
| M376 | R | | 2.43 | 2.06 | 1.19 | 1.06 | 0.94 | 1.02 | 1.11 | 0.93 | 1.24 | 0.96 |
| M376 | W | | 3.45 | 2.82 | 0.85 | 0.65 | 0.54 | 0.71 | 0.76 | 0.98 | 0.80 | 8.22 |
| M376 | Y | G593D | 2.64 | 1.36 | 0.54 | 0.67 | 0.28 | 0.44 | 0.62 | 1.00 | 1.71 | 6.39 |
| D377 | F | | 3.28 | 2.42 | 0.94 | 1.05 | 0.93 | 0.91 | 0.98 | 1.04 | 1.10 | 0.88 |
| D377 | H | | 3.88 | 2.65 | 1.24 | 1.18 | 1.13 | 1.23 | 1.05 | 1.27 | 1.80 | 1.07 |
| D377 | T | | 1.80 | | 0.84 | 1.06 | 0.78 | 0.79 | 0.86 | | | |
| D377 | Y | | 3.50 | 2.26 | 0.91 | 0.89 | 0.91 | 0.87 | 0.84 | 1.16 | 1.01 | 0.95 |
| D377 | G | A287S | 8.91 | 5.57 | 2.67 | 2.44 | 2.45 | 1.93 | 2.00 | 1.50 | 2.71 | 1.74 |
| L378 | E | | 0.56 | | 1.10 | | | | | | | |
| L378 | K | | 2.87 | 2.39 | 0.98 | 0.96 | 1.16 | 0.83 | 0.82 | 1.02 | 1.50 | 0.73 |
| L378 | R | | 2.91 | 2.23 | 1.00 | 0.95 | 1.06 | 0.79 | 0.95 | 0.95 | 1.85 | 0.42 |
| S380 | K | | 3.58 | 2.81 | 1.96 | 1.71 | 1.82 | 1.13 | 1.60 | 0.96 | 1.88 | 0.67 |
| S380 | R | | 3.90 | 2.66 | 1.38 | 1.29 | 1.64 | 1.13 | 1.17 | 1.16 | 1.55 | 0.70 |
| S381 | R | | 2.75 | 1.97 | 0.90 | 0.87 | 1.02 | 0.91 | 0.89 | 0.99 | 1.23 | 0.95 |
| F393 | S | | 2.72 | 1.77 | 0.48 | 0.52 | 0.72 | 0.47 | 0.50 | 0.96 | 0.49 | 2.30 |
| R394 | C | | 0.17 | | 0.39 | | | | | | | |
| R394 | E | | 0.30 | | 1.01 | | | | | | | |
| R394 | G | | | | | 1.05 | 0.54 | | | | | |
| R394 | M | | | | | 0.91 | 0.65 | | | | | |
| R394 | P | | 1.11 | 1.04 | 0.31 | 0.37 | 0.25 | 0.36 | 0.36 | 0.58 | 0.09 | 3.47 |
| A396 | D | K524R | 2.94 | 2.19 | 0.98 | 0.58 | 0.31 | 0.71 | 0.96 | 0.99 | 0.88 | 1.82 |
| G399 | V | | 1.43 | 1.21 | 0.08 | 0.02 | 0.02 | 0.18 | 0.39 | 1.08 | 1.32 | 1.04 |
| G399 | V | S101I | 1.76 | 1.04 | 0.05 | 0.02 | 0.01 | 0.16 | 0.38 | 0.33 | 1.03 | 0.99 |
| G399 | V | W693L | 1.57 | 1.22 | 0.20 | 0.24 | 0.15 | 0.49 | 0.38 | 0.51 | 1.85 | 0.30 |
| E403 | H | | 1.10 | | 0.89 | 0.88 | 0.65 | 0.74 | 0.74 | | | |
| E403 | L | | 1.30 | | 0.82 | 0.72 | 0.46 | 0.66 | 0.79 | | | |
| E403 | S | | 1.17 | | 0.69 | 0.63 | 0.53 | 0.65 | 0.65 | | | |
| T404 | D | | 0.74 | | 1.90 | | | | | | | |
| T404 | D | | 0.55 | | 1.38 | | | | | | | |
| T404 | F | | | | | 0.31 | 0.23 | | | | | |
| A405 | T | E419F | 1.37 | 1.24 | 0.21 | 0.18 | 0.23 | 0.22 | 0.24 | 0.67 | 0.45 | 0.12 |
| Y413 | D | | 0.45 | | 0.46 | 0.36 | 0.35 | 0.40 | 0.31 | | | |
| N415 | A | | 0.63 | | 0.83 | 0.75 | 0.56 | 0.72 | 0.60 | | | |
| G416 | R | | 2.92 | 2.084 | 1.04 | 0.93 | 1.26 | 0.64 | 0.99 | 0.917 | 0.886 | 0.347 |
| G416 | W | | 4.13 | 3.145 | 0.84 | 0.97 | 1.21 | 0.94 | 0.56 | 1.209 | 1.634 | 1.414 |
| N417 | W | | 1.14 | 1.24 | 0.22 | 0.24 | 0.35 | 0.44 | 0.19 | 0.64 | 0.65 | 1.06 |
| E419 | L | | 3.86 | 2.71 | 0.79 | 0.73 | 0.74 | 0.68 | 0.85 | 1.18 | 0.64 | 0.45 |
| E419 | M | | 4.56 | 2.95 | 0.92 | 0.88 | 0.85 | 0.72 | 0.85 | 1.43 | 0.71 | 0.41 |
| E419 | F | | 2.11 | 1.67 | 0.67 | 0.67 | 0.76 | 0.57 | 0.66 | 1.04 | 0.72 | 0.27 |
| E419 | F | A405T | 1.37 | 1.24 | 0.21 | 0.18 | 0.23 | 0.22 | 0.24 | 0.67 | 0.45 | 0.12 |
| E419 | K | I485V | 3.94 | 2.60 | 0.94 | 0.90 | 0.94 | 0.64 | 1.01 | 1.25 | 0.71 | 0.25 |
| V422 | M | | 0.81 | | 0.42 | 0.51 | 0.42 | 0.46 | 0.38 | | | |
| A468 | S | E533A | 1.47 | 1.43 | 0.41 | 0.42 | 0.57 | 0.53 | 0.44 | 0.87 | 0.59 | 0.48 |
| I485 | V | E419K | 3.94 | 2.60 | 0.94 | 0.90 | 0.94 | 0.64 | 1.01 | 1.25 | 0.71 | 0.25 |
| I510 | D | | 0.62 | | 2.00 | | | | | | | |
| I510 | E | | 0.53 | | 1.34 | | | | | | | |
| I510 | F | | 0.88 | | 1.46 | | | | | | | |
| I510 | R | | 3.75 | 2.55 | 1.81 | 1.61 | 1.51 | 1.39 | 1.52 | 1.11 | 1.43 | 0.96 |
| L514 | K | | 3.21 | 2.40 | 1.32 | 1.24 | 1.26 | 1.10 | 1.25 | 1.13 | 1.60 | 1.31 |
| L514 | F | Q518P | 0.90 | | 0.70 | 0.58 | 0.58 | 0.60 | 0.57 | | | |
| F516 | R | | 1.06 | | 0.64 | 0.68 | 0.64 | 0.64 | 0.72 | | | |
| S517 | D | | 0.60 | | 1.20 | | | | | | | |
| S517 | F | | 1.57 | | 0.63 | 0.83 | 0.67 | 0.78 | 0.55 | | | |
| S517 | R | | 1.99 | 1.64 | 0.93 | 0.81 | 0.99 | 0.65 | 0.78 | 1.12 | 0.95 | 0.40 |
| S517 | W | | 4.16 | 2.84 | 0.88 | 1.15 | 1.17 | 1.46 | 0.77 | 1.04 | 1.23 | 2.76 |
| S517 | Y | | 1.93 | | 0.79 | 0.99 | 0.85 | 1.09 | 0.70 | | | |
| Q518 | R | | 1.28 | | 0.98 | 0.91 | 0.75 | 0.94 | 0.94 | | | |
| Q518 | P | | 1.06 | | 0.81 | 0.83 | 0.76 | 0.71 | 0.70 | | | |
| Q518 | P | L514F | 0.90 | | 0.70 | 0.58 | 0.58 | 0.60 | 0.57 | | | |
| Q521 | R | | 0.84 | | 0.55 | 0.44 | 0.43 | 0.47 | 0.53 | | | |
| Q521 | E | L522Y | 0.48 | | 1.24 | | | | | | | |
| L522 | Y | Q521E | 0.48 | | 1.24 | | | | | | | |
| K524 | R | A396D | 2.94 | 2.19 | 0.98 | 0.58 | 0.31 | 0.71 | 0.96 | 0.99 | 0.88 | 1.82 |
| E533 | F | | 1.00 | | 0.66 | 0.64 | 0.61 | 0.77 | 0.83 | | | |
| E533 | A | A468S | 1.47 | 1.43 | 0.41 | 0.42 | 0.57 | 0.53 | 0.44 | 0.87 | 0.59 | 0.48 |
| E533 | R | G195V | 1.24 | | 0.50 | 0.67 | 0.53 | 0.58 | 0.61 | | | |
| W534 | C | S536W | | | | 0.58 | 0.20 | | | | | |
| S536 | G | | 1.32 | 1.12 | 0.37 | 0.45 | 0.26 | 0.41 | 0.52 | 0.90 | 0.40 | 0.13 |
| S536 | P | | 3.15 | | 2.82 | 3.36 | 1.29 | 1.91 | 2.76 | | | |
| S536 | R | | 3.85 | 2.36 | 0.44 | 0.62 | 0.45 | 0.52 | 0.93 | 1.05 | 0.92 | 0.27 |
| S536 | E | | 0.26 | | 1.37 | 1.27 | 0.33 | | | | | |
| S536 | E | Q624H | | | | 1.70 | 0.41 | | | | | |
| S536 | W | W543C | | | | 0.58 | 0.20 | | | | | |
| G537 | E | | 0.45 | | 1.82 | | | | | | | |
| G537 | T | | 2.13 | 1.86 | 0.82 | 1.00 | 0.71 | 0.97 | 1.08 | 1.17 | 1.21 | 0.66 |
| V540 | C | | | | | 0.39 | 0.24 | | | | | |
| V540 | E | | 0.29 | | 2.93 | 1.40 | 0.39 | | | | | |
| V540 | F | | 0.52 | | 1.17 | 0.88 | 0.42 | | | | | |
| V540 | G | | 0.22 | | 1.52 | | | | | | | |
| Y545 | S | | 0.75 | | 0.59 | 0.57 | 0.56 | 1.21 | 0.59 | | | |
| Y545 | V | H211N | 0.64 | | 0.81 | 0.70 | 0.25 | 0.41 | 0.65 | | | |
| S546 | D | | | | | 2.32 | 1.39 | | | | | |
| S546 | E | | 0.43 | | 2.27 | 2.45 | 1.42 | | | | | |
| S546 | I | | 1.08 | 1.16 | 0.34 | 0.44 | 0.47 | 0.55 | 0.36 | 0.83 | 0.76 | 0.91 |
| S546 | R | | | | | 0.96 | 0.51 | | | | | |
| S546 | W | | 1.73 | | 1.09 | 1.26 | 0.52 | 1.02 | 0.60 | | | |
| S546 | Y | R102S | | | | 0.57 | 1.39 | | | | | |
| S547 | D | | | | | 1.88 | 1.20 | | | | | |
| S547 | E | | 0.47 | | 2.34 | | | | | | | |
| S547 | F | | 1.89 | | 2.04 | 1.89 | 1.99 | 1.95 | 1.96 | | | |
| S547 | F | L323F | 3.00 | 2.06 | 1.35 | 1.54 | 1.45 | 1.44 | 0.88 | 1.03 | 1.14 | 0.53 |
| S547 | G | | 0.85 | | 1.37 | | | | | | | |
| S547 | K | R735H | | | | 0.28 | 0.53 | | | | | |
| G548 | C | | 0.39 | | 0.67 | | | | | | | |
| G548 | E | | 0.39 | | 1.67 | | | | | | | |
| G548 | R | | 2.79 | 1.80 | 1.37 | 0.72 | 1.49 | 0.99 | 1.16 | 1.16 | 0.54 | 0.61 |
| G548 | W | | 0.79 | | 2.45 | | | | | | | |
| D590 | F | | 8.79 | 5.20 | 0.99 | 0.24 | 0.15 | 0.84 | 1.51 | 1.57 | 2.72 | 3.94 |
| D590 | M | | 5.79 | 3.59 | 0.91 | 0.17 | 0.09 | 0.52 | 1.57 | 1.59 | 2.03 | 1.70 |
| D590 | W | | 5.40 | 3.22 | 0.72 | 0.16 | 0.05 | 0.36 | 1.23 | 1.54 | 1.79 | 1.75 |
| D591 | E | | 0.40 | | 0.94 | | | | | | | |
| D591 | L | | 0.78 | | 1.13 | | | | | | | |
| N592 | P | | 2.83 | 1.92 | 1.27 | 0.74 | 0.35 | 1.02 | 1.49 | 1.48 | 0.91 | 1.48 |
| G593 | V | | 4.58 | 2.99 | 1.10 | 0.62 | 0.41 | 0.57 | 1.28 | 1.46 | 2.15 | 5.43 |
| G593 | D | M376Y | 2.64 | 1.36 | 0.54 | 0.67 | 0.28 | 0.44 | 0.62 | 1.00 | 1.71 | 6.39 |
| F596 | P | | 6.44 | 4.03 | 0.34 | 0.06 | 0.02 | 0.25 | 0.93 | 1.38 | 1.93 | 7.53 |
| G600 | D | | 2.80 | 2.30 | 0.70 | 0.76 | 0.28 | 0.55 | 1.02 | 1.03 | 1.43 | 4.46 |
| G600 | V | | 1.36 | 1.16 | 0.13 | 0.08 | 0.03 | 0.07 | 0.47 | 0.71 | 0.73 | 1.42 |
| G600 | W | | 1.91 | 1.56 | 0.03 | 0.02 | 0.01 | 0.05 | 0.46 | 0.78 | 0.85 | 1.95 |
| W606 | S | | 0.97 | | 0.77 | 0.48 | 0.36 | 0.64 | 0.75 | | | |
| Q624 | H | S536E | | | | 1.70 | 0.41 | | | | | |
| G645 | Q | | 5.43 | 3.22 | 1.70 | 1.14 | 0.54 | 1.15 | 1.48 | 1.42 | 1.85 | 1.20 |
| A649 | G | | 0.36 | | 0.74 | | | | | | | |
| V692 | M | | 2.47 | 2.11 | 0.78 | 0.81 | 1.17 | 1.18 | 0.67 | 1.42 | 0.69 | 2.88 |
| W693 | C | | 0.86 | | 0.39 | 0.65 | 0.74 | 0.37 | 0.53 | | | |
| W693 | F | | 4.18 | 2.66 | 0.81 | 0.88 | 1.71 | 1.19 | 0.59 | 1.60 | 1.99 | 1.43 |
| W693 | N | | 0.94 | | 0.46 | 0.66 | 0.61 | 0.49 | 0.43 | | | |
| W693 | Q | | 1.55 | | 0.43 | 0.76 | 0.66 | 0.68 | 0.60 | | | |
| W693 | V | | 1.86 | | 0.42 | 0.78 | 0.94 | 0.45 | 0.55 | | | |
| W693 | L | | 4.72 | 3.08 | 1.05 | 0.88 | 2.15 | 1.22 | 0.75 | 1.66 | 2.11 | 0.16 |
| W693 | L | G399V | 1.57 | 1.22 | 0.20 | 0.24 | 0.15 | 0.49 | 0.38 | 0.51 | 1.85 | 0.30 |
| Y697 | G | | 0.68 | | 0.27 | 0.24 | 0.29 | 0.35 | 0.27 | | | |
| Y701 | G | | 1.82 | | 1.18 | 0.73 | 1.15 | 1.14 | 1.13 | | | |
| Y701 | R | | 2.21 | 1.58 | 0.62 | 0.67 | 0.56 | 0.50 | 0.80 | 1.01 | 1.11 | 0.34 |
| N704 | E | | 0.42 | | 0.73 | | | | | | | |
| N704 | G | | | | | 0.86 | 0.60 | | | | | |
| N704 | R | | 0.80 | 1.05 | 0.29 | 0.19 | 0.17 | 0.25 | 0.46 | 0.64 | 0.65 | 0.21 |
| N704 | W | | 0.57 | 0.78 | 0.12 | 0.13 | 0.17 | 0.21 | 0.12 | 0.54 | 0.44 | 0.21 |
| S705 | R | | 2.00 | 1.44 | 0.48 | 0.41 | 0.29 | 0.42 | 0.75 | 0.88 | 0.64 | 0.26 |
| T708 | K | | 1.17 | 1.08 | 0.45 | 0.18 | 0.16 | 0.24 | 0.70 | 0.59 | 0.66 | 0.08 |
| D709 | K | | 4.54 | 2.90 | 0.39 | 0.32 | 0.45 | 0.34 | 0.64 | 0.80 | 0.47 | 0.12 |
| D709 | V | | 0.84 | 0.84 | 0.19 | 0.13 | 0.17 | 0.17 | 0.30 | 0.56 | 0.34 | 0.08 |
| V710 | F | | 0.48 | | 0.86 | | | | | | | |
| S712 | H | | 1.49 | 1.31 | 1.11 | 1.15 | 1.17 | 1.14 | 1.01 | 1.14 | 0.81 | 0.86 |
| S712 | L | | | | | 0.26 | 0.48 | | | | | |
| S712 | G | D219A | | | | 0.35 | 1.00 | | | | | |
| G714 | H | | | | | 0.93 | 0.33 | | | | | |
| G714 | K | | 6.91 | 3.99 | 0.81 | 0.55 | 0.19 | 0.44 | 1.31 | 0.75 | 0.48 | 0.19 |
| G714 | V | | 0.26 | | 1.56 | | | | | | | |
| I718 | L | | 3.25 | 2.21 | 0.61 | 0.58 | 0.98 | 1.20 | 0.35 | 1.55 | 1.30 | 1.55 |
| I718 | V | | 2.53 | | 1.17 | 1.11 | 1.79 | 1.63 | 1.05 | | | |
| R735 | H | S547K | | | | 0.28 | 0.53 | | | | | |
| K745 | N | | 0.61 | | 0.33 | 0.30 | 0.38 | 0.36 | 0.21 | | | |

remark: R102Q is a control, known from literature

Unless indicated otherwise, the amino acid positions identified herein relate to those in full-length wild-type neprilysin (minus the initiating methionine), as disclosed in SEQ ID NO: 1. Thus, for example, 5100 refers to the Serine at position 100 in full length wild-type neprilysin.

Another embodiment of the present invention is a protease variant which is derived from human neprilysin having an at least 2-, 5-, 10-, 15-, 20-, 30-, 40-, 50-, 100-, 200-fold increased specificity against a certain substrate or an at least 2-, 5-, 8-, 10-, 15-, 20-, 25-, 50-fold increased activity against a certain substrate compared to wild type human neprilysin. In a preferred embodiment the foregoing increase in specificity is at least 10-fold. In a further preferred embodiment the foregoing increase in activity is at least 4-fold. In a particular embodiment, the protease variant has increased specificity or activity for Aβ.

Another embodiment of the present invention is a protease variant which is derived from human neprilysin having an at least 2-, 5-, 10-, 15-, 20-, 30-, 40-, 50-, 100-, 200-fold increased specificity against a first neprilysin substrate relative to a second neprilysin substrate compared to wild-type neprilysin, or an at least 2-, 5-, 8-, 10-, 15-, 20-, 25-, 50-fold increased activity against a first neprilysin substrate relative to a second Neprilysin substrate compared to wild type human neprilysin. In a preferred embodiment the foregoing increase in specificity is at least 10-fold. In a further preferred embodiment the foregoing increase in activity is at least 4-fold. In a particular embodiment, the protease variant has increased specificity or activity for Aβ. In further embodiments the first neprilysin substrate is Aβ and the second neprilysin substrate is selected from the group consisting of: Angiotensin-1 and -2, ANP, BNP, bradykinin, Endothelin-1 and -2, Neuropeptide Y, Neurotensin, Adrenomedullin, Bombesin, BLP, CGRP, Enkephalins, FGF-2, fMLP, GRP, Neurokinin A, Neuromedin C, Oxytocin, PAMP, Substance P and VIP. In still further embodiments the first neprilysin substrate is Aβ and the second neprilysin substrate is selected from the group consisting of: Angiotensin-1 and -2, ANP, BNP, bradykinin, Endothelin-1 and -2, Neuropeptide Y and Neurotensin.

Another embodiment of the present invention is a protease variant which is derived from human neprilysin having an at least 2-, 5-, 10-, 15-, 20-, 30-, 40-, 50-, 100-, 200-fold increased specificity against a certain substrate and an at least 2-, 5-, 8-, 10-, 15-, 20-, 25-, 50-fold increased activity against the aforementioned substrate compared to wild type human Neprilysin. In a preferred embodiment the foregoing increase in specificity is at least 10-fold. In a further preferred embodiment the foregoing increase in activity is at least 4-fold. In a particular embodiment, the protease variant has increased specificity and activity for Aβ.

Yet another embodiment is a protease variant which is derived from human neprilysin having at least one alteration in the sequence selected from the group consisting of T99, 5100, 5101, G104, D107, G195, T206, H211, H214, H217, D219, Q220, G224, 5227, 8228, D229, F247, A287, 8292, L323, Y346, M376, D377, L378, 5380, 5381, F393, R394, A396, G399, E403, T404, A405, Y413, N415, G416, N417, E419, V422, A468, I485, I510, L514, F516, S517, Q518, Q521, L522, K524, E533, W534, 5536, G537, V540, Y545, 5546, 5547, G548, D590, D591, N592, G593, F596, G600, G600, W606, Q624, G645, A649, V692, W693, Y697, Y701, N704, 5705, T708, D709, V710, 5712, G714, R735 and K745. In a particular embodiment the alteration at any of the recited positions is a substitution of the native residue by another naturally occurring amino acid. A further embodiment is an aforementioned protease variant wherein the substitution leads to an increased specificity and/or activity against a certain substrate compared to human wild-type neprilysin. A further preferred embodiment is an aforementioned protease variant having an at least 2-, 4-, 5-, 10-, 20-, 30-, 40-, 50-, 75-, 100-, 200-fold increased specificity against a certain substrate compared to wild-type human neprilysin. A further preferred embodiment is an aforementioned protease variant having, in addition to increased specificity, an at least 2-, 3-, 4-, 5-, 8-, 10-, 15-, 20-, 25-, 50-fold increased activity against a certain substrate compared to wild-type human neprilysin. In particular embodiments, the protease variant has increased specificity or activity for Aβ.

Yet another embodiment is a protease variant which is derived from human neprilysin having at least one alteration in the sequence selected from the group consisting of T99, 5100, 5101, G104, D107, G195, T206, H211, H214, H217, D219, Q220, G224, S227, R228, D229, F247, A287, 8292, L323, Y346, M376, D377, L378, 5380, 5381, F393, R394, A396, G399, E403, T404, A405, Y413, N415, G416, N417, E419, V422, A468, I485, I510, L514, F516, S517, Q518, Q521, L522, K524, E533, W534, S536, G537, V540, Y545, S546, S547, G548, D590, D591, N592, G593, F596, G600, G600, W606, Q624, G645, A649, V692, W693, Y697, Y701, N704, 5705, T708, D709, V710, 5712, G714, R735 and K745. In a particular embodiment, said alteration is substitution by another naturally occurring amino acid. A further embodiment is an aforementioned protease variant having an at least 2-, 4-, 5-, 10-, 20-, 30-, 40-, 50-, 75-, 100-, 200-fold increased specificity against a certain substrate compared to wild-type human neprilysin and having, in addition to increased specificity, an at least 2-, 3-, 4-, 5-, 8-, 10-, 15-, 20-, 25-, 50-fold increased activity against the aforementioned substrate compared to wild type human neprilysin. In a particular embodiment, the substrate against which the protease variant has increased specificity or activity is Aβ.

A further embodiment is a protease variant which is derived from human neprilysin having at least one alteration in the sequence selected from the group consisting of T99 by D, S100 by I, S101 by L, V, Y, or I, G104 by L, M, R, V or W, D107 by N, V or W, G195 by V, T206 by R, H211 by N, H214 by N, H217 by N, D219 by A, Q220 by K, G224 by W, S227 by L or R R228 by G, D229 by N, F247 by C or L, A287 by S, R292 by M, L323 by F, Y346 by W, M376 by Y, D377 by F, H, T, Y or G, L378 by E, K or R, S380 by K or R, S381 by R, F393 by S, R394 by C, E, G, M or P, A396 by D, G399 by V, E403 by H, L or S T404 by D or F A405 by T, Y413 by D, N415 by A, G416 by R or W N417 by W, E419 by L, M, F or K, V422 by M, A468 by S, 1485 by V, 1510 by D, E, F or R, L514 by K or F, F516 by R, S517 by D, F, R, W or Y, Q518 by R or P, Q521 by R or E, L522 by Y, K524 by R, E533 by F, A or R, W534 by C, S536 by G, P, R, E, or W, G537 by E or T, V540 by C, E, F or G, Y545 by S or V, S546 by D, E, I, R, W or Y, S547 by D, E, F, G or K, G548 by C, E, R or W, D590 by F, M or W, D591 by E or L, N592 by P, G593 by V or D, F596 by P, G600 by D, V or W, W606 by S, Q624 by H, G645 by Q, A649 by G, V692 by M, W693 by C, F, N, Q, V or L, Y697 by G, Y701 by G or R, N704 by E, G, R or W, S705 by R, T708 by K, D709 by K or V V710 by F, S712 by H, L, Q or G, G714 by H or K, R735 by H and K745 by N.

Another embodiment is a protease variant, which is derived from human neprilysin wherein R102 is replaced by another naturally occurring amino acid other than Gln (O) and/or 1718 is replaced by another naturally occurring amino acid other than Ala (A). A further embodiment is a protease variant which is derived from human neprilysin wherein at one or more positions the following exchanges of amino acids are introduced: R102 by C, L, M, P, S or W and/or 1718 by L or V.

Yet another embodiment is a protease variant according to any of the aforementioned variants having an altered specificity against at least one substrate selected from the group consisting of Amyloid β₄₀, Amyloid β₄₂, Angiotensin-1 and -2, ANP, BNP, bradykinin, Endothelin-1 and -2, Neuropeptide Y, Neurotensin, Adrenomedullin, Bombesin, BLP, CGRP, Enkephalins, FGF-2, fMLP, GRP, Neurokinin A, Neuromedin C, Oxytocin, PAMP, Substance P or VIP. A further embodiment is a protease variant according to any of the aforementioned variants having an altered specificity against at least one substrate selected from the group consisting of Amyloid β₄₀ Amyloid β₄₂/Angiotensin-1 and -2, ANP, BNP, bradykinin, Endothelin-1 and -2, Neuropeptide Y or Neurotensin. A further embodiment is a protease variant according to any of the aforementioned variants having an altered specificity against at least Amyloid β₄₀ or Amyloid β₄₂.

Neprilysin Variants with Increased Specificity for Aβ.

One embodiment of the present invention is a protease variant which is derived from human neprilysin having an at least 10-fold increased specificity against a certain substrate compared to wild type human neprilysin.

With respect to neprilysin variants with increased specificity for Aβ, the inventors have determined that one or more amino acid substitution mutations at the following positions (relative to wild-type neprilysin depicted in SEQ ID NO: 1): 101, 107, 220, 224, 227, 228, 229, 247, 287, 323, 376, 377, 378, 380, 381, 393, 394, 396, 399, 405, 416, 417, 419, 468, 485, 510, 514, 517, 524, 533, 536, 537, 546, 547, 548, 590, 592, 593, 596, 600, 645, 692, 693, 701, 704, 705, 708, 709, 712, 714 and 718 exhibit enhanced specificity for Aβ versus a panel of peptide substrates, compared to wild-type neprilysin. Mutant/variant neprilysin polypeptides possessing an amino acid substitution at one or more the following positions: 227, 228, 247, 399, 419, 590, 593, 596, 600, 709, 714 and 718 (relative to the position in SEQ ID NO: 1), were especially more specific for Aβ than certain other peptides.

In another aspect there is provided an isolated neprilysin variant which comprises a sequence disclosed in SEQ ID NO:1, or a fragment thereof, but with an amino acid substitution at one or more positions in SEQ ID NO: 1 selected from position: 101, 107, 220, 224, 227, 228, 229, 247, 287, 323, 376, 377, 378, 380, 381, 393, 394, 396, 399, 405, 416, 417, 419, 468, 485, 510, 514, 517, 524, 533, 536, 537, 546, 547, 548, 590, 592, 593, 596, 600, 645, 692, 693, 701, 704, 705, 708, 709, 712, 714 and 718. In a particular embodiment said polypeptide has an amino acid substitution at one or more positions in SEQ ID NO: 1 selected from position: 227, 228, 247, 399, 419, 590, 593, 596, 600, 709, 714 and 718.

Variant forms of neprilysin with one or more of the following specific substitutions have been made and shown to possess enhanced specificity for Aβ than certain other peptides: S227R, S227L, R228G, F247L, F247C, G339V, E419M, E419L, D590W, D590M, D590F, G593V, F596P, G600W, G600V, G600D, G600L, G645Q, D709K, D709V, G714K; or 1718L. Each of these variant polypeptides are particular embodiments of the invention.

The inventors have found that mutant neprilysin polypeptides that comprise just one amino acid substitution at an identified location possess enhanced specificity for Aβ. However, of the mutants/variants generated, those that include two or more substitution, in particular those with at least two substitutions being at positions 399 and 714 were especially specific for Aβ relative to any of the off-peptide substrates, when compared to wild-type neprilysin. Accordingly, in separate embodiments the variant neprilysin forms posses one, two, three, four, five, six, seven, eight or more amino acid substitutions relative to the human neprilysin depicted in SEQ ID NO: 1. For example, a particular variant polypeptide is one that comprises the G399V and G714K substitutions.

According to a further aspect of the invention there is provided an isolated neprilysin variant polypeptide which compared to wild type neprilysin having the sequence according to the position in SEQ ID NO: 1, possesses an amino acid other than Glycine (G) at position 399 and/or an amino acid other than Glycine (G) at position 714, and optionally one or more substitutions relative to wild type neprilysin. In a particular embodiment the one or more optional substitutions are at any of the following positions: 227, 228, 247, 419, 590, 593, 596, 600, 645, 709 or 718, with particular substitutions being any of: S227R, S227L, R228G, F247L, F247C, E419M, E419L, D590W, D590M, D590F, G593V, F596P, G600W, G600V, G600D, G600L, G645Q, D709K, D709V or 1718L.

According to a further aspect of the invention there is provided an isolated neprilysin variant polypeptide which compared to wild type neprilysin having the sequence according to the position in SEQ ID NO: 1, possesses a valine (V) at position 399 and/or a lysine (K) at position 714, and optionally one or more substitutions relative to wild type neprilysin. In particular embodiments the one or more optional substitutions are selected from the group consisting of: S227R, S227L, R228G, F247L, F247C, E419M, E419L, D590W, D590M, D590F, G593V, F596P, G600W, G600V, G600D, G600L, G645Q, D709K, D709V and 1718L. In one particular embodiment the one or more optional substitutions are selected from the group consisting of: S227R, R228G, F247L, E419M, D590M, D590F, G593V, F596P, G600V, G600D, G600L, G645Q and D709V. In further embodiments, the isolated neprilysin variant polypeptide which compared to wild type neprilysin having the sequence according to the position in SEQ ID NO: 1, possesses a valine (V) at position 399 and a lysine (K) at position 714, and one or more optional substitutions at one or more of the following positions: 227, 228, 247, 419, 590, 593, 596, 600, 645, 709, and 718, particular substitutions being any of: S227R, S227L, R228G, F247L, F247C, E419M, E419L, D590W, D590M, D590F, G593V, F596P, G600W, G600V, G600D, G600L, G645Q, D709K, D709V and 1718L.

According to a further aspect of the invention there is provided an isolated neprilysin variant polypeptide disclosed in any of tables 3, 5, 7 or 9. In particular, any mutant neprilysin polypeptide selected from B1 to B12, C1 to C23 and D1 to D10.

One embodiment of the invention is a protease variant according to any of the aforementioned variants wherein the human neprilysin is a soluble human neprilysin or a derivative thereof.

Another embodiment encompasses a nucleic acid encoding an aforementioned protease variant. A further embodiment is a vector comprising the aforementioned nucleic acid. Yet, another embodiment is a host cell comprising the aforementioned vector, such as one into which the vector has been transformed or transfected

One embodiment is a method for producing a protease variant, wherein the method comprises the following steps: culturing the aforementioned host cell comprising the vector housing the nucleic acid encoding the Neprilysin variant, under conditions suitable for the expression of the protease variant; and recovering the protease variant from the host cell culture.

In some embodiments, the protease variant or derivative thereof or nucleic acid encoding same is isolated. An isolated biological component (such as a nucleic acid molecule or protein such as a protease) is one that has been substantially separated or purified away from other biological components in the cell of the organism in which the component naturally occurs, e.g., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

One embodiment is a pharmaceutical composition comprising an aforementioned protease variant. A further embodiment is a pharmaceutical composition comprising an aforementioned protease variant and a pharmaceutical acceptable carrier.

One embodiment is a method for treating a human neprilysin substrate related disease comprising the step of: administering to a patient in need thereof a therapeutically effective amount or dose of an aforementioned protease variant, whereby symptoms of the human neprilysin substrate related disease is ameliorated. Examples of such neprilysin substrate related diseases are Dementia (Alzheimer disease), wherein in the substrate is amyloid beta, neuropathic pain, wherein the substrate is bradykinin, cardiovascular diseases, wherein the substrate is angiotensin, or cancer, wherein the substrate is neurotensin.

Another embodiment is use of an aforementioned protease variant for the production of a medicament for the treatment of a human neprilysin substrate related disease. In a preferred embodiment the human neprilysin substrate related disease is a disease wherein the abundance of the aforementioned substrate leads to the disease, e.g. Aβ-related pathologies. Examples of such neprilysin substrate related diseases are Dementia (Alzheimer disease), wherein in the substrate is amyloid beta, neuropathic pain, wherein the substrate is bradykinin, cardiovascular diseases, wherein the substrate is angiotensin, or cancer, wherein the substrate is neurotensin.

Stipulating the location of the substitution position relative to human neprilysin full-length sequence (minus initiating methionine) (SEQ ID NO: 1) allows identification of the corresponding position in extracellular human neprilysin and in neprilysin (full length or extracellular domain) from other species, including rat and mouse. In addition to full-length Neprilysin variants, the invention also encompasses fragments of full-length neprilysin which fragments contain the amino acid substitution(s) indicated herein, and possess the ability to cleave one or more of the substrate peptides that wild-type neprilysin cleaves. Particularly fragments would be those that arise following proteolytic cleavage of full-length protein, e.g. the extracellular region etc.

Thus according to one aspect of the invention there is provided an isolated polypeptide which compared to wild type neprilysin, has at least 10-fold greater specificity for cleavage of amyloid beta than for cleavage of one or more of the substrates selected from ANP, BNP, angiotensin-1, bradykinin, endothelin 1, neuropeptide Y and neurotensin. In one embodiment the isolated peptide has at least 10-fold greater specificity for cleavage of Aβ than each of the peptides selected from: ANP, BNP, angiotensin-1, bradykinin, endothelin 1, neuropeptide Y, neurotensin, adrenomedullin and insulin b-chain. In another embodiment, the isolated polypeptide (neprilysin variant) has at least 2-fold reduced specificity for cleavage against each of the substrates selected from ANP, BNP, angiotensin-1, bradykinin, endothelin 1, neuropeptide Y, neurotensin, adrenomedullin and insulin b-chain, compared to wild type neprilysin having the sequence disclosed in SEQ ID NO: 1. In another embodiment, the neprilysin variant has at least 10-fold increased specificity for cleavage of amyloid beta than for cleavage of one or more of the substrates selected from ANP, BNP, angiotensin-1, bradykinin, endothelin 1, neuropeptide Y and neurotensin and at least 5-fold reduced specificity for cleavage against each of the substrates selected from ANP, BNP, angiotensin-1, bradykinin, endothelin 1, neuropeptide Y, neurotensin, adrenomedullin and insulin b-chain compared to wild type neprilysin having the sequence disclosed in SEQ ID NO: 1.

According to another aspect of the invention there is provided an isolated neprilysin variant polypeptide having at least 3-, 4-, 5-, 6-, 8-, 10, 15-, 20-fold greater activity for cleavage of amyloid beta compared to wild-type neprilysin having the sequence disclosed in SEQ ID NO: 1.

Nucleic acids encoding the isolated polypeptides of the invention, plasmid vectors housing such nucleic acids, host cells capable of expressing such polypeptides also form aspects of the invention. Other aspects of the invention include: A method for reducing amyloid β peptide concentration, said method comprising administration of the isolated polypeptide of the invention, or a fusion protein comprising said polypeptide; as well as, a pharmaceutical composition capable of degrading amyloid β peptide, comprising a pharmaceutically acceptable amount of the isolated neprilysin variant or fusion protein comprising the neprilysin variant of the invention, together with a pharmaceutically acceptable carrier or excipient; as well as, a method of prevention and/or treatment of a condition wherein degradation of amyloid β peptide is beneficial, such as Alzheimer's disease, comprising administering to a mammal, including man in need of such prevention and/or treatment, a therapeutically effective amount of the isolated neprilysin variant or fusion protein comprising the neprilysin variant of the invention; as well the use of a fusion protein comprising an isolated neprilysin variant of the invention in medical therapy; as well as the use of an isolated neprilysin variant or fusion protein comprising the neprilysin variant of the invention, in the manufacture of a medicament for prevention and/or treatment of conditions wherein of degradation of amyloid β peptide is beneficial, e.g. Alzheimer's disease and mild cognitive impairment.

In another aspect of the present invention, there is provided a modified neprilysin variant protein M-A, wherein A is a neprilysin variant polypeptide as described herein and M is an attached moiety that prolongs the half-life of the neprilysin polypeptide.

As used herein, the M-A molecule (modified Neprilysin variant) will also be referred to as a fusion protein.

In a particular embodiment, the attached moiety M is another polypeptide, such that M-A is a fusion protein of the neprilysin variant fused to a second polypeptide.

When M is another polypeptide (M polypeptide), preferably it is attached at the N-terminus of the neprilysin variant. In a particular embodiment the M polypeptide is attached to the N-terminus of the neprilysin variant of the invention.

In one aspect of the present invention, there is provided a fusion protein, wherein M is an Fc part of an antibody. In one embodiment of this aspect, said antibody is an IgG antibody.

In another embodiment of this aspect, said antibody is an IgG1 antibody.

In another aspect of the present invention, there is provided a fusion protein, wherein M is human serum albumin (HSA) or a HSA binding domain or peptide or a variant HSA with one or more mutations, preferably the variant HSA is C34S .

In another aspect of the present invention, there is provided a fusion protein, wherein M is transferrin.

In another aspect of the present invention, there is provided a fusion protein, wherein M is an unstructured amino acid polymer.

In another aspect of the present invention, there is provided a fusion protein, wherein M is an antibody-binding domain.

In another aspect of the present invention, there is provided a fusion protein, wherein M and A are linked together with a linker, L.

In another aspect of the present invention, there is provided a fusion protein, wherein L is selected from a peptide and a chemical linker.

In certain embodiments the fusion protein is made up of two protein or peptide component parts fused or joined together. However, as used herein, the term fusion protein can mean a protein to which a modulator is fused, said modulator need not itself be a protein.

Thus, in other aspect of the present invention, the attached modulator is pegylation and/or glycosylation.

In another aspect of the present invention, there is provided a method for reducing Aβ peptide concentration, said method comprising administration of a neprilysin variant with increased specificity for Aβ as taught herein. In one embodiment of this aspect, said reduction of Aβ peptide is accomplished in plasma. In another embodiment of this aspect, said reduction of Aβ peptide is accomplished in cerebrospinal fluid (CSF). In yet another embodiment of this aspect, said reduction of Aβ peptide is accomplished in CNS.

In another aspect of the present invention, there is provided a pharmaceutical composition capable of degrading Aβ peptide, comprising a pharmaceutically acceptable amount of a neprilysin variant with increased specificity for Aβ as taught herein, or a fusion protein comprising said variant according to the invention together with a pharmaceutically acceptable carrier or excipient.

In another aspect of the present invention, there is provided a method of prevention and/or treatment of a condition wherein of degradation of Aβ peptide is beneficial, comprising administering to a mammal, including man in need of such prevention and/or treatment, a therapeutically effective amount of a neprilysin variant with increased specificity for Aβ or a fusion protein according to the invention.

In another aspect of the present invention, there is provided a method of prevention and/or treatment of Alzheimer's disease or other neurodegenerative disease mediated by or associated with amyloid beta plaque formation comprising administering to a mammal, including man in need of such prevention and/or treatment, a therapeutically effective amount of a neprilysin variant with increased specificity for Aβ or a fusion protein according to the invention.

In another aspect of the present invention, there is provided a neprilysin variant with increased specificity for Aβ or a fusion protein according to the invention for use in medical therapy.

In another aspect of the present invention, there is provided use of a neprilysin variant with increased specificity for Aβ or a fusion protein of the invention, for the prevention and/or treatment of conditions wherein of degradation of Aβ peptide is beneficial.

In another aspect of the present invention, there is provided use of a neprilysin variant with increased specificity for Aβ or a fusion protein of the invention, in the manufacture of a medicament for prevention and/or treatment of conditions wherein of degradation of Aβ peptide is beneficial.

In another aspect of the present invention, there is provided use of a neprilysin variant with increased specificity for Aβ or a fusion protein of the invention for the prevention and/or treatment of Alzheimer's disease or mild cognitive impairment. In one embodiment of this aspect, said medicament reduces Aβ peptide concentration. Said reduction of Aβ peptide being accomplished in plasma, CSF and/or CNS.

In another aspect of the present invention, there is provided use of a neprilysin variant with increased specificity for Aβ or a fusion protein of the invention, in the manufacture of a medicament for prevention and/or treatment of Alzheimer's disease or mild cognitive impairment. In one embodiment of this aspect, said medicament reduces Aβ peptide concentration. Said reduction of Aβ peptide is accomplished in plasma, CSF and/or CNS.

In some embodiments, the neprilysin variant with increased specificity for Aβ, or derivative thereof, or nucleic acid encoding it is isolated.

The neprilysin variants of the present invention may be derived or based on the full length neprilysin protein, or on the extra-cellular part of the protein which houses the regions capable of peptide cleavage. The extra-cellular part is defined as the part of neprilysin that is defined as outside the membrane region. The invention also comprises smaller fragments of neprilysin as long as the catalytic activity is preserved against the Aβ peptide.

A neprilysin variant polypeptide or derivative thereof provided herein can be prepared by recombinant expression of nucleic acid sequences encoding the same in a host cell. To express a neprilysin variant polypeptide or derivative thereof recombinantly, a host cell can be transfected with one or more recombinant expression vectors carrying DNA fragments encoding the neprilysin or derivative thereof such that the neprilysin or derivative is expressed in the host cell. Standard recombinant DNA methodologies are used to prepare and/or obtain nucleic acids encoding the neprilysin or derivative thereof; to incorporate these nucleic acids into recombinant expression vectors; and, to introduce the vectors into host cells, such as those described in Sambrook, Fritsch and Maniatis (eds), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989).

In general, the neprilysin variants described herein have pharmacological activity resulting from their ability to process/degrade pharmacological active substrates. An altered activity and/or specificity by a factor of two is sufficient to change the pharmacological activity of the variant compared to wild type. The activity/specificity of the neprilysin variants can be determined by assays known in the art. In vivo assays are known in the art and further described in the examples section.

Another embodiment of the present invention refers to a molecule that is composed of one part that binds Aβ peptide with high affinity. This affinity is below micromolar in binding affinity. The binding affinity for Aβ peptide is preferably at nanomolar in binding affinity. The other part that is involved in the interaction with Aβ peptide is an active component that cleaves the Aβ peptide at one or more site in the structure of the Aβ peptide. The reason to combine a binding part linked together with a catalytic active part that both recognize the Aβ peptide is that the binding part binds the Aβ peptide and thereby increase the local concentration (the binding part and the catalytic part) is binding to the dissociated form of Aβ peptide. Some bind specifically to the dissociated form without binding to the aggregated form. Some bind to both aggregated and dissociated forms. Some such antibodies bind to a naturally occurring short form of Aβ (i.e. covalently or in another way linked together) of Aβ peptide to become cleaved by the active part that is locally around due to the linkage engineered in the bifunctional molecule. The linkage between the Aβ peptide binding component and the Aβ peptide-degrading component is preferably mediated by the plasma half-life modulator component with or without a linker component.

In some embodiments of this invention the therapeutic agents include fusion proteins that specifically bind to Aβ peptide or other component of amyloid plaques. Such compound can be a part of a monoclonal or polyclonal or any other Aβ peptide-binding agent. These compounds bind to Aβ peptide with a binding affinity greater than or equal to about 10⁶, 10⁷, 10⁸, 10⁹, or 10¹⁰M⁻¹. These binding components are preferably connected with an Aβ peptide-degrading component.

One aspect of the invention refers to the combination with the “Fc” domain of an antibody with a Aβ peptide degrading component in the fusion protein. Antibodies comprise two functionally independent parts, a variable domain known as “Fab”, which binds antigen, and a constant domain known as “Fc”, which links to such effector functions as complement activation and attack by phagocytic cells. An Fc has a long serum half-life, whereas a Fab is short-lived (Capon et al. (1989), Nature 337: 525-31). When constructed together with a therapeutic protein, an Fc domain can provide longer half-life or incorporate such functions as Fc receptor binding, protein A binding, complement fixation and perhaps even placental transfer.

Preferred molecules in accordance with this invention are Fc-linked amyloid β peptide degrading protein such as neprilysin-related proteins.

Useful modifications of protein therapeutic agents by fusion with the Fc domain of an antibody are discussed in detail in a publication entitled, “Modified Peptides as Therapeutic Agents (WO 99/25044). That publication discusses linkage to a “vehicle” such as PEG, dextran, or an Fc region. Linking to the C-terminal part of an Fc domain has been described in the literature as a possible approach (Protein Eng. 1998 11:495-500). This allows a N-terminal linkage on the protein part of the fusion protein. This invention describes this approach and the beneficial effect of using this strategy obtaining a fusion protein with optimized properties for in vivo efficacy.

IgG molecules interact with four classes of Fc receptors, namely FcγRI, FcγRII, FcγRIII and FcRn. In preferred embodiments, the immunoglobulin (Ig) component of the fusion protein has at least a portion of the constant region of an IgG that enables binding to FcRn. In one aspect of the invention, the binding affinity of fusion proteins for one of the FcγR family of receptors is reduced by using heavy chain isotypes, or variants thereof as, fusion partners that have reduced binding affinity for Fc receptors on cells. Thus, in a preferred embodiment, an antibody-based fusion protein with enhanced in vivo circulating half-life is obtained by linking the Fc domain of an IgG to a second non-immunoglobulin protein.

In one embodiment, the Aβ-peptide degrading component of the fusion protein is an enzyme. The term “enzyme” is used herein to describe proteins, analogs thereof, and fragments thereof, which are active as proteases or peptidases. Preferably, enzymes include serine, aspartic, metallo and cysteine proteases. Preferably, the fusion protein of the present invention displays enzymatic biological activity.

In another embodiment, the immunoglobulin domain is selected from the group consisting of the Fc domain of IgG, the heavy chain of IgG, and the light chain of IgG. In another embodiment, the constant region of the antibody in the fusion protein will be of human origin, and belong to the immunoglobulin family derived from the IgG class of immunoglobulins, in particular from classes IgG1, IgG2, IgG3 or IgG4. It is also alternatively possible to use constant regions of immunoglobulins belonging to the IgG class from other mammals, in particular from rodents or primates; however, it is also possible, according to the invention, to use constant regions of the immunoglobulin classes IgD, IgM, IgA or IgE. Typically, the antibody fragments that are present in the construct according to the invention will comprise the Fc domain CH₃, or parts thereof, and at least one part segment of the Fc domain CH₂. Alternatively, it is also possible to conceive of fusion constructs according to the invention which contain, as component (A), the CH₃ domain and the hinge region, for the dimerization.

However, it is also possible to use derivatives of the immunoglobulin sequences that are found in the native state, in particular those variants that contain at least one replacement, deletion and/or insertion (combined here under the term “variant”). Typically, such variants possess at least 90%, preferably at least 95%, and more preferably at least 98%, sequence identity with the native sequence. Variants, which are particularly preferred in this context, are replacement variants that typically contain less than 10, preferably less than 5, and very particularly preferably less than 3, replacements as compared with the respective native sequence. Attention is drawn to the following replacement possibilities as being preferred: Trp with Met, Val, Leu, Ile, Phe, His or Tyr, or vice versa; Ala with Ser, Thr, Gly, Val, Ile or Leu, or vice versa; Glu with Gln, Asp or Asn, or vice versa; Asp with Glu, Gln or Asn, or vice versa; Arg with Lys, or vice versa; Ser with Thr, Ala, Val or Cys, or vice versa; Tyr with His, Phe or Trp, or vice versa; Gly or Pro with one of the other 19 native amino acids, or vice versa.

Soluble receptor-IgG fusion proteins are common immunological reagents and methods for their construction are known in the art (see e.g., U.S. Pat. No. 5,225,538). A functional Aβ peptide-degrading domain may be fused to an immunoglobulin Fc domain derived from an immunoglobulin class or subclass. The Fc domains of antibodies belonging to different Ig classes or subclasses can activate diverse secondary effector functions. Activation occurs when the Fc domain is bound by a cognate Fc receptor. Secondary effector functions include the ability to activate the complement system, to cross the placenta, and to bind various microbial proteins. The properties of the different classes and subclasses of immunoglobulins are described in Roitt et al., Immunology, p. 4.8 (Mosby—Year Book Europe Ltd., 3d ed. 1993). The Fc domains of antigen-bound IgG1, IgG3 and IgM antibodies can activate the complement enzyme cascade. The Fc domain of IgG2 appears to be less effective, and the Fc domains of IgG4, IgA, IgD and IgE are ineffective at activating complement. Thus one can select an Fc domain based on whether its associated secondary effector functions are desirable for the particular immune response or disease being treated with the Aβ peptide degrading-Fc fusion protein. If it would be advantageous to harm or kill target cells, one could select an especially active Fc domain (IgG1) to make the Aβ peptide degrading-Fc-fusion protein. Alternatively, if it would be desirable to produce the Aβ peptide degrading-Fc-Fusion without triggering the complement system, an inactive IgG4 Fc domain could be selected. This invention describes a fusion protein with a catalytic component linked to a Fc part and not a direct binding component. This means that the effect and activity from the Fc will be limited because many Fc effects are mediated through the binding. For example complement activation is dependent on binding and the formation of a network.

C-terminally of the immunoglobulin fragment, a fusion construct according to the invention typically, but not necessarily, contains a transition region between catalytic and modulator part, which transition region can in turn contain a linker sequence, with this linker sequence preferably being a peptide sequence. This peptide sequence can have a length from between 1 and up to 70 amino acids, where appropriate even more amino acids, preferably from 10 to 50 amino acids, and particularly preferably between 12 and 30 amino acids. The linker region of the transition sequence can be flanked by further short peptide sequences which can, for example, correspond to DNA restriction cleavage sites. Any restriction cleavage sites with which the skilled person is familiar from molecular biology can be used in this connection. Suitable linker sequences are preferably artificial sequences, which contain a high number of proline residues (for example at every second position in the linker region) and, in addition to that, preferably have an overall hydrophilic character. A linker sequence, which consists of at least 30% of proline residues, is preferred. The hydrophilic character can preferably be achieved by means of at least one amino acid having a positive charge, for example lysine or arginine, or negative charge, for example aspartate or glutamate. Overall, the linker region therefore also preferably contains a high number of glycine and/or proline residues in order to confer on the linker region the requisite flexibility and/or rigidity.

However, native sequences, for example those fragments of ligands belonging to the neprilysin family which are disposed extracellularly, but immediately act, i.e. in front of, the cell membrane, are also suitable for use as linkers, where appropriate after replacement, deletion or insertion of the native segments as well. These fragments are preferably the 50 amino acids which follow extracellularly after the transmembrane region or else subfragments of these first 50 amino acids. However, preference is given to these segments having at least 85% sequence identity with the corresponding natural human sequences, with very particular preference being given to at least 95% sequence identity and particular preference being given to at least 99% sequence identity in order to limit the immunogenicity of these linker regions in the fusion protein according to the invention and not elicit any intrinsic humoral defense reaction. Within the context of the present invention, the linker region should preferably not possess any immunogenicity.

However, as an alternative to peptide sequences, which are linked to the Aβ peptide degrading component and the plasma half-life modulator component, by way of amide-like bonds, it is also possible to use compounds which are of a nonpeptide or pseudopeptide nature or are based on noncovalent bonds. Examples which may be mentioned in this connection are, in particular, N-hydroxysuccinimide esters and heterobifunctional linkers, such as N-succinimidyl-3-(2-pyridyldi-thio) propionate (SPDP) or similar crosslinkers.

Other ways of regulating the plasma half-life is to use pegylation or other type of modifications that increasing the molecular weight such as glycosylation.

As noted above, polymer modulators may also be used. Various means for attaching chemical moieties useful as modulator are currently available, see, e.g., patent application WO 96/11953, entitled “N-Terminally Chemically Modified Protein Compositions and Methods” herein incorporated by reference in its entirety. This PCT publication discloses, among other things, the selective attachment of water-soluble polymers to the N-terminus of proteins.

A preferred polymer modulator is polyethylene glycol (PEG). The PEG group may be of any convenient molecular weight and may be linear or branched. The average molecular weight of the PEG will preferably range from about 2 kiloDalton (“kD”) to about 100 kDa, more preferably from about 5 kDa to about 50 kDa, most preferably from about 5 kDa to about 10 kDa. The PEG groups will generally be attached to the compounds of the invention via acylation or reductive alkylation through a reactive group on the PEG moiety (e.g., an aldehyde, amino, thiol, or ester group) to a reactive group on the compound (e.g. an aldehyde, amino, or ester group).

A useful strategy for the PEGylation of protein consists of combining, through forming a conjugate linkage in solution, a protein and a PEG moiety, each bearing a special functionality that is mutually reactive toward the other. The protein can be prepared with conventional recombinant expression techniques. The proteins are “preactivated” with an appropriate functional group at a specific site. The precursors are purified and fully characterized prior to reacting with the PEG moiety. Ligation of the protein with PEG usually takes place in aqueous phase and can be easily monitored by reverse phase analytical HPLC. The PEGylated protein can be easily purified by preparative HPLC and characterized by analytical HPLC, amino acid analysis and laser desorption mass spectrometry.

Polysaccharide polymers are another type of water-soluble polymer which may be used for protein modification. Dextrans are polysaccharide polymers comprised of individual subunits of glucose predominantly linked by α1 -6 linkages. The dextran itself is available in many molecular weight ranges, and is readily available in molecular weights from about 1 kD to about 70 kD. Dextran is a suitable water-soluble polymer for use in the present invention as a modulator by itself or in combination with another modulator (e.g., Fc), see e.g. WO 96/11953 and WO 96/05309. The use of dextran conjugated to therapeutic or diagnostic immunoglobulins has been reported; see, for example, European Patent Publication EP 0 315 456, which is hereby incorporated by reference. Dextran of about 1 kD to about 20 kD is preferred when dextran is used as a vehicle in accordance with the present invention.

Carbohydrate (oligosaccharide) groups may conveniently be attached to sites that are known to be glycosylation sites in proteins. Generally, O-linked oligosaccharides are attached to serine (Ser) or threonine (Thr) residues while N-linked oligosaccharides are attached to asparagine (Asn) residues when they are part of the sequence Asn-X-Ser/Thr, where X can be any amino acid except proline. X is preferably one of the 19 naturally occurring amino acids other than proline. The structures of N-linked and O-linked oligosaccharides and the sugar residues found in each type are different. One type of sugar that is commonly found on both is N-acetylneuraminic acid (referred to as sialic acid). Sialic acid is usually the terminal residue of both N-linked and O-linked oligosaccharides and, by virtue of its negative charge, may confer acidic properties to the glycosylated compound. Such site(s) may be incorporated in the linker of the compounds of this invention and are preferably glycosylated by a cell during recombinant production of the polypeptide compounds (e.g., in mammalian cells such as CHO, BHK, COS). However, such sites may further be glycosylated by synthetic or semi-synthetic procedures known in the art Amino acids that are suitable for glycosylation can be incorporated at specific sites both in the modulator and the protein part. Preferable techniques to use for engineering these specific amino acids are site-directed mutagenesis or comparable method.

Other possible modifications include hydroxylation of proline and lysine, phosphorylation of hydroxyl groups of seryl or threonyl residues, oxidation of the sulfur atom in Cys, methylation of the alpha-amino groups of lysine, arginine, and histidine side chains. Creighton, Proteins: Structure and Molecule Properties (W. H. Freeman & Co., San Francisco), pp. 79-86 (1983). Thus, glycosylation sites in the Aβ peptide-degrading component can be engineered. For example, residues preferably on the surface of neprilysinrilysin structure are modified to allow the glycosylation. The 3D structure of neprilysinrilysin is know and can be used to select suitable amino acid replacement for the introduction of both glycosylation and pegylation sites. Glycosylation sites are introduced using for example the Asn-X-Ser/Thr sequence. For pegylation, suitable surface exposed amino acids are for example replaced to cysteine residues for specific and efficient coupling of the pegylation component.

Compounds of the present invention may be changed at the DNA level, as well. The DNA sequence of any portion of the compound may be changed to codons more compatible with the chosen host cell. For E. coli, which is the preferred host cell, optimized codons are known in the art. Codons may be substituted to eliminate restriction sites or to include silent restriction sites, which may aid in processing of the DNA in the selected host cell. The vehicle, linker and peptide DNA sequences may be modified to include any of the foregoing sequence changes.

Linkers: Any “linker” group is optional. When present, its chemical structure is not critical, since it serves primarily as a spacer. The linker is preferably made up of amino acids linked together by peptide bonds. Thus, in preferred embodiments, the linker is made up of from 1 to 20 amino acids linked by peptide bonds, wherein the amino acids are selected from the 20 naturally occurring amino acids. Some of these amino acids may be glycosylated, as is well understood by those in the art. In a more preferred embodiment, the 1 to 20 amino acids are selected from glycine, alanine, proline, asparagine, glutamine, and lysine. Even more preferably, a linker is made up of a majority of amino acids that are sterically unhindered, such as glycine and alanine. Thus, preferred linkers are polyglycines (particularly (Gly)₄, (Gly)₅), poly(Gly-Ala), and polyalanines. A particularly useful linker is (Gly)₅Ser or (Gly)₄Ser.

The quantitative specificity of proteases varies over a wide range. There are very unspecific proteases known, such as papain which cleaves all polypeptides that contain a phenylalanine, a valine or an leucine residue, or trypsin which cleaves all polypeptides that contain an arginine or a lysine residue. On the other hand, there are highly specific proteases known, such as the tissue-type plasminogen activator (t-PA) which cleaves plasminogen only at a single specific sequence. Proteases with high substrate specificity play an important role in the regulation of protein functions in living organisms. The specific cleavage of polypeptide substrates, for example, activates precursor proteins or deactivates active proteins or enzymes, thereby regulating their functions. Several proteases with high substrate specificities are used in medical applications. Pharmaceutical examples for activation or deactivation by cleavage of specific polypeptide substrates are the application of t-PA in acute cardiac infarction, which activates plasminogen to resolve fibrin clots, or the application of Ancrod in stroke which deactivates fibrinogen, thereby decreasing blood viscosity and enhancing its transport capacity. While t-PA is a human protease with an activity necessary in human blood regulation, Ancrod is a non-human protease. It was isolated from the viper Agkistrodon rhodostoma, and comprises the main ingredient of the snake's poison. Therefore, there exist a few non-human proteases with therapeutic applicability. Their identification, however, is usually highly incidental.

The treatment of diseases by administering drugs is typically based on a molecular mechanism initiated by the drug that activates or inactivates a specific protein function in the patient's body, be it an endogenous protein or a protein of an infecting microbe or virus. While the action of chemical drugs on these targets is still difficult to understand or to predict, protein drugs are able to specifically recognize these target proteins among millions of other proteins. Prominent examples of proteins that have the intrinsic possibility to recognize other proteins are antibodies, receptors, and proteases. Although there are a huge number of potential target proteins, only very few proteases are available today to address these target proteins. Due to their proteolytic activity, proteases are particularly suited for the inactivation of protein or peptide targets. When considering human proteins only, the number of potential target proteins is yet enormous. It is estimated that the human genome comprises between 30,000 and 100,000 genes, each of which encodes a different protein. Many of these proteins or peptides are involved in human diseases and are therefore potential pharmaceutical targets. It might be unlikely to find such a protease with a particular qualitative specificity by screening natural isolates. Therefore there is a need to optimize the catalytic selectivity of a known protease or other scaffold proteins including catalytic antibodies.

Selection systems for proteases of known specificity are known in the art, for instance, from Smith et al., Proc. Natl. Acad. Sci. USA, Vol. 88 (1991). As exemplified, the system comprises the yeast transcription factor GAL4 as the selectable marker, a defined and cleavable target sequence inserted into GAL4 in conjunction with the TEV protease. The cleavage separates the DNA binding domain from the transcription activation domain and therewith renders the transcription factor inactive. The phenotypical inability of the resulting cells to metabolize galactose can be detected by a calorimetric assay or by the selection on the suicide substrate 2-deoxygalactose.

Further, selection may be performed by the use of peptide substrates with modifications as, for example, fluorogenic moieties based on groups as ACC, previously described by Harris et al. (US 2002/022243).

Identical or similar approaches could be used in order to identify or produce an effective amyloid β peptide-degrading component as described in this invention. That starting point for the engineering of this amyloid β peptide-degrading component could be an enzyme that possesses some activity against amyloid β peptide or that have no activity at all. Other components could be a scaffold protein where specific regions are randomized to possess activity against the amyloid β peptide. There are described various scaffold proteins in the literature where one part of the scaffold structure is the core structure holding the randomized part in a relative fixed positions to generate a binding or active site. Enzymes that possess some activity against amyloid β peptide could be natural proteases that are described to degrade amyloid β peptide. For example, Neprilysin could be engineered either by rationale design or a more random approach to become more efficient as a amyloid β peptide-degrading component.

Laboratory techniques to generate proteolytic enzymes with altered sequence specificities are in principle known. They can be classified by their expression and selection systems. Genetic selection means to produce a protease or any other protein within an organism which protease or any other protein is able to cleave a precursor protein which in turn results in an alteration of the growth behaviour of the producing organism. From a population of organisms with different proteases those having an altered growth behaviour can be selected. This principle was reported by Davis et al. (U.S. Pat. No. 5,258,289). The production of a phage system is dependent on the cleavage of a phage protein, which is activated in the presence of a proteolytic enzyme, or antibody which is able to cleave the phage protein. Selected proteolytic enzymes, scaffolds or antibodies would have the ability to cleave an amino acid sequence for activation of phage production.

A system to generate proteolytic enzymes with altered sequence specificities with membrane-bound proteases is reported. Iverson et al. (WO 98/49286) describe an expression system for a membrane-bound protease that is displayed on the surface of cells. An essential element of the experimental design is that the catalytic reaction has to be performed at the cell surface, i.e., the substrates and products must remain associated with the bacterium expressing the enzyme at the surface. Another example of a selection system is the use of FACS sorting (Varadarajan et al., Proc. Natl. Acad. Sci. USA, Vol. 102, 6855 (2005)) that express the active protein on a cell surface and sort cells that contains variants with improved properties. They showed a three million-fold change in specificity for a protease cleavage site.

A system to generate proteolytic enzymes with altered sequence specificities with self-secreting proteases is also known. Duff et al. (WO 98/11237) describe an expression system for a self-secreting protease. An essential element of the experimental design is that the catalytic reaction acts on the protease itself by an autoproteolytic processing of the membrane-bound precursor molecule to release the matured protease from the cellular membrane into the extracellular environment.

Broad et al. (WO 99/11801) disclose a heterologous cell system suitable for the alteration of the specificity of proteases. The system comprises a transcription factor precursor wherein the transcription factor is linked to a membrane-anchoring domain via a protease cleavage site. The cleavage at the protease cleavage site by a protease releases the transcription factor, which in turn initiates the expression of a target gene being under the control of the respective promotor. The experimental design of alteration of the specificity consists in the insertion of protease cleavage sites with modified sequences and the subjection of the protease to mutagenesis.

According to the invention, any protein or peptide can be used directly or as a starting point to generate a suitable amyloid β peptide-degrading component. For example, according to the invention, any protease can be used as first protease. Preferably, any protein or peptide that are of human origin is used. If a natural protein or peptide, normally existing in the human body, is used, the smallest possible changes are preferred.

In some methods, two or more fusion proteins with different binding specificities and/or degradation activity are administered simultaneously, in which case the dosage of each fusion protein administered falls within the ranges indicated. Fusion protein is usually administered on multiple occasions. Intervals between single dosages can be, for example, weekly, monthly, every three months or yearly. Intervals can also be irregular as indicated by measuring blood levels of fusion protein in the plasma of the patient. In some methods, dosage is adjusted to achieve a plasma fusion protein concentration of 1-1000 ug/ml and in some methods 25-300 ug/ml. Also in some methods, dosage is adjusted to achieve a plasma fusion protein concentration of 1-1000 ng/ml and in some methods 25-300 ng/ml. Alternatively, fusion protein can be administered as a sustained release formulation, in which case less frequent administration is required. Dosage and frequency vary depending on the half-life of the fusion protein in the patient. In general, fusion protein with an Fc part shows a long half-life. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime. It is predicted that a catalytic active amyloid β peptide degrading fusion protein can be administrated at a lower dose compare to a binding agent such as for example an antibody.

Actual dosage levels of the active ingredients in the pharmaceutical compositions of the present invention may be varied so as to obtain an amount of the active ingredient, which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

All publications or patents cited herein are entirely incorporated herein by reference as they show the state of the art at the time of the present invention and/or to provide description and enablement of the present invention. Publications refer to any scientific or patent publications, or any other information available in any media format, including all recorded, electronic or printed formats. The following references are entirely incorporated herein by reference: Ausubel, et al., ed., Current Protocols in Molecular Biology, John Wiley & Sons, Inc., NY, N.Y. (1987-2001); Sambrook, et al., Molecular Cloning: A Laboratory Manual, 2^{nd} Edition, Cold Spring Harbor, N.Y. (1989); Harlow and Lane, antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, N.Y., (1997-2001).

It is an object of the present invention to provide methods and materials, which are suited for the development of a treatment for neurodegenerative diseases and for the identification of compounds useful for therapeutic intervention in such diseases. The invention provides a method for preventing and treating neurodegenerative disorders comprising administering to the peripheral system of a mammalian an effective amount of an optimized enzymatic active compound. In particular, the enzymatic active compound is a fusion protein where one part has enzymatic activity and the other part regulate the half-life in plasma. The method is suited for preventing and treating brain amyloidosis such as Alzheimer's disease. The invention also provides different assay principles—biochemical and in particular cellular assays for testing an optimized enzymatic compound, preferably screening a plurality of compounds, for modulating activity and plasma half-life.

In a further embodiment, the assay comprises the addition of a known inhibitor of the member of the Neprilysin family before detecting said enzymatic activity. Suitable inhibitors are e.g. phosphoramidon, thiorphan, spinorphin, or a functional derivative of the foregoing substances.

In a general sense, assays according to the invention measure the enzymatic activity and half-life in plasma, both in vitro and in vivo.

In another aspect, the present invention provides a method for producing a medicament comprising the steps of (i) identifying a compound which degrades Aβ-peptides, preferably a compound that is highly specific and with high Aβ-peptides degrading activity (ii) linking this Aβ-peptides degrading compound to a modulator compound that determine the half-time in plasma.

Further aspects of the invention include nucleic acid molecules that comprise nucleotide sequences that encode variant neprilysin polypeptides of the present invention, vectors, in particular plasmid vectors, which contain such nucleic acids, and host cells comprising nucleic acids that encode the variant neprilysin polypeptides of the invention.

According to another aspect of the present invention there is provided an isolated nucleic acid molecule comprising a nucleotide sequence that encodes a neprilysin variant with enhanced selectivity for Aβ relative to an off-target peptide substrate, and/or relative to wild type human neprilysin, which variant possess one or more amino acid substitutions located at positions: 101, 107, 220, 224, 227, 228, 229, 247, 287, 323, 376, 377, 378, 380, 381, 393, 394, 396, 399, 405, 416, 417, 419, 468, 485, 510, 514, 517, 524, 533, 536, 537, 546, 547, 548, 590, 592, 593, 596, 600, 692, 693, 701, 704, 705, 708, 709, 712, 714 and 718, relative to the position in SEQ ID NO: 1.

According to a further aspect of the present invention there is provided an isolated nucleic acid molecule comprising a nucleotide sequence that encodes a neprilysin variant with enhanced specificity for Aβ relative to an off-target (non-Aβ) peptide substrate, and/or relative to wild type human neprilysin, which variant possess one or more amino acid substitutions located at positions: 227, 228, 247, 399, 419, 590, 593, 596, 600, 709, 714 and 718, relative to the position in SEQ ID NO: 1. Particular variants have one or both of residues at positions 399 and 714 substituted for a non-wild type codon. The wild type codons are those present in SEQ ID NO: 1.

The introduction of a mutation into the polynucleotide sequence to exchange one nucleotide for another nucleotide optionally resulting in a mutation in the corresponding polypeptide sequence may be accomplished by site-directed mutagenesis using any of the methods known in the art. Such techniques are explained in the literature, for example: Ausubel et al., eds., Current Protocols in Molecular Biology, John Wiley & Sons, New York, N.Y. (2002).

Particularly useful is the procedure that utilizes a super coiled, double stranded DNA vector with the polynucleotide sequence of interest and two polynucleotide primers harboring the mutation of interest. The primers are complementary to opposite strands of the vector and are extended during a thermocycling reaction using, for example, Pfu DNA polymerase. On incorporation of the primers, a mutated plasmid containing nicks is generated. Subsequently, this plasmid is digested with DpnI, which is specific for methylated and hemimethylated DNA to digest the start plasmid without destroying the mutated plasmid (see Example 2.1).

Other procedures know in the art for creating, identifying and isolating mutants may also be used, such as, for example, gene shuffling or phage display techniques.

According to another aspect of the invention there are provided isolated polynucleotides (including genomic DNA, genomic RNA, cDNA and mRNA; double stranded as well as +ve and −ve strands), which encode the polypeptides of the invention.

The polynucleotides can be synthesised chemically, or isolated by one of several approaches known to the person skilled in the art such as polymerase chain reaction (PCR) or ligase chain reaction (LCR) or by cloning from a genomic or cDNA library.

Once isolated or synthesised, a variety of expression vector/host systems may be used to express neprilysin variant polypeptides. These include, but are not limited to microorganisms such as bacteria expressed with plasmids, cosmids or bacteriophage; yeasts transformed with expression vectors; insect cell systems transfected with baculovirus expression systems; plant cell systems transfected with plant virus expression systems, such as cauliflower mosaic virus; or mammalian cell systems (for example those transfected with adenoviral vectors); selection of the most appropriate system is a matter of choice.

Expression vectors usually include an origin of replication, a promoter, a translation initiation site, optionally a signal peptide, a polyadenylation site, and a transcription termination site. These vectors also usually contain one or more antibiotic resistance marker gene(s) for selection. As noted above, suitable expression vectors may be plasmids, cosmids or viruses such as phage or retroviruses. Examples of suitable retroviral vectors that could be used include: pLNCX2 (Clontech, BD Biosciences, Cat#631503), pVPac-Eco (Stratagene, Cat#217569) or pFB-neo (Statagene, Cat#217561). Examples of packaging cell lines that might be used with these vectors include: BD EcoPack2-293 (Clontech, BD Biosciences, Cat#631507), BOSC 23 (ATCC, CRL-11270), or Phoenix-Eco (Nolan lab, Stanford University). The coding sequence of the polypeptide is placed under the control of an appropriate promoter (i.e. HSV, CMV, TK, RSV, SV40 etc), control elements and transcription terminator so that the nucleic acid sequence encoding the polypeptide is transcribed into RNA in the host cell transformed or transfected by the expression vector construct. The coding sequence may or may not contain a signal peptide or leader sequence for secretion of the polypeptide out of the host cell. Preferred vectors will usually comprise at least one multiple cloning site. In certain embodiments there will be a cloning site or multiple cloning site situated between the promoter and the gene of interest. Such cloning sites can be used to create N-terminal fusion proteins by cloning a second nucleic acid sequence into the cloning site so that it is contiguous and in-frame with the gene of interest. In other embodiments there may be a cloning site or multiple cloning site situated immediately downstream of the gene of interest to facilitate the creation of C-terminal fusions in a similar fashion to that for N-terminal fusions described above, may be expressed in a variety of hosts such as bacteria, plant cells, insect cells, fungal cells and human and animal cells. Eukaryotic recombinant host cells are particularly suitable. Examples include yeast, mammalian cells including cell lines of human, bovine, porcine, monkey and rodent origin, and insect cells including Drosophila, army fallworm and silkworm derived cell lines. A variety of mammalian expression vector/host systems may be used to express the neprilysin variant polypeptides of the present invention. Particular examples include those adapted for expression using a recombinant adenoviral, adeno-associated viral (AAV) or retroviral system. Vaccinia virus, cytomegalovirus, herpes simplex virus, and defective hepatitis B virus systems, amongst others may also be used. Particular cell lines derived from mammalian species which may be used and which are commercially available include, L cells L-M(TK-) (ATCC CCL 1.3), L cells L-M (ATCC CCL 1.2), HEK 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26) and MRC-5 (ATCC CCL 171).

Although it is preferred that mammalian expression systems are used for expression of the neprilysin variant polynucleotide sequence, it will be understood that other vector and host cell systems such as, bacterial, yeast, plant, fungal, insect are also possible.

The vectors containing the DNA coding for the neprilysin variant polypeptides of the invention can be introduced into host cells to express a polypeptide of the present invention via any one of a number of techniques, including calcium phosphate transformation, DEAE-dextran transformation, cationic lipid mediated lipofection, electroporation or infection. Performance of the invention is neither dependent on nor limited to any particular strain of host cell or vector; those suitable for use in the invention will be apparent to, and a matter of choice for, the person skilled in the art.

Host cells genetically modified to include a variant neprilysin encoding nucleotide sequence may be cultured under conditions suitable for the expression and recovery of the encoded proteins from the cell culture. Such expressed proteins/polypeptides may be secreted into the culture medium or they may be contained intracellularly depending on the sequences used, i.e. whether or not suitable secretion signal sequences were present.

Expression and purification of the polypeptides of the invention can be easily performed using methods well known in the art (for example as described in Sambrook et al., ibid).

Thus, in another aspect, the invention provides for cells and cell lines transformed or transfected with the nucleic acids of the present invention. The transformed cells may, for example, be mammalian, bacterial, yeast or insect cells. According to a further aspect of the invention there is provided a host cell adapted to express a neprilysin variant polypeptide of the present invention.

A plasmid comprising a nucleotide sequence encoding a neprilysin variant of the present invention represents a further aspect of the invention.

Suitable expression systems can also be employed to create transgenic animals capable of expressing a variant neprilysin (see for example, U.S. Pat. No. 5,714,666).

According to a further aspect of the invention there is provided a transgenic, non-human animal whose cells comprise a nucleic acid encoding a variant neprilysin with increased specificity for Aβ, and regulatory control sequences capable of directing expression of the gene in said cells. In a preferred embodiment the transgenic animal is murine, ovine or bovine.

According to a further aspect of the invention there is provided a host cell adapted to express a variant neprilysin polypeptide of the invention from the nucleic acid sequence of the invention. Preferred host cells are mammalian such as CHO-K1 or Phoenix cells. Human cells are most preferred for expression purposes.

The compounds of this invention may be made in transformed host cells using recombinant DNA techniques. To do so, a recombinant DNA molecule coding for the fusion protein is prepared. Methods of preparing such DNA molecules are well known in the art. For instance, sequences coding for the modulator and protein could be excised from DNA using suitable restriction enzymes. Alternatively, the DNA molecule could be synthesized using chemical synthesis techniques, such as the phosphoramidate method. Also, a combination of these techniques could be used.

The invention also includes a vector capable of expressing the modulator, protein or fusion in an appropriate host. The vector comprises the DNA molecule that codes for the modulator, protein and/or fusion operatively linked to appropriate expression control sequences. Methods of effecting this operative linking, either before or after the DNA molecule is inserted into the vector, are well known. Expression control sequences include promoters, activators, enhancers, operators, ribosomal binding sites, start signals, stop signals, cap signals, polyadenylation signals, and other signals involved with the control of transcription or translation.

The resulting vector having the DNA molecule thereon is used to transform an appropriate host. This transformation may be performed using methods well known in the art.

Any of a large number of available and well-known host cells may be used in the practice of this invention. The selection of a particular host is dependent upon a number of factors recognized by the art. These include, for example, compatibility with the chosen expression vector, toxicity of the fusion encoded by the DNA molecule, rate of transformation, ease of recovery of the fusion, expression characteristics, bio-safety and costs. A balance of these factors must be struck with the understanding that not all hosts may be equally effective for the expression of a particular DNA sequence. Within these general guidelines, useful microbial hosts include bacteria (such as E. coli sp.), yeast (such as Saccharomyces sp.) and other fungi, insects, plants, mammalian (including human) cells in culture, or other hosts known in the art.

Next, the transformed host is cultured and purified. Host cells may be cultured under conventional fermentation conditions so that the desired compounds are expressed. Such fermentation conditions are well known in the art. Finally, the fusion is purified from culture by methods well known in the art. One preferably approach is to use Protein A or similar technique to purify the fusion protein when using a Fc part as a modulator.

The modulator, protein and fusion may also be made by synthetic methods. For example, solid phase synthesis techniques may be used. Suitable techniques are well known in the art, and include those described in Merrifield (1973), Chem. Polypeptides, pp. 335-61 (Katsoyannis and Panayotis eds.); Merrifield (1963), J. Am. Chem. Soc. 85: 2149; Davis et al. (1985), Biochem. Intl. 10: 394-414; Stewart and Young (1969), Solid Phase Peptide Synthesis; U.S. Pat. No. 3,941,763; Finn et al. (1976), The Proteins (3rd ed.) 2: 105-253; and Erickson et al. (1976), The Proteins (3rd ed.) 2: 257-527. Solid phase synthesis is the preferred technique of making individual peptides or proteins since it is the most cost-effective method of making small peptides or proteins.

In general, the compounds of this invention have pharmacologic activity resulting from their ability to degrade the amyloid β peptide in vivo. The activity of these compounds can be measured by assays known in the art. For the Fc-neprilysin compounds, in vivo assays are further described in the Examples section herein.

In general, the present invention also provides the possibility of using pharmaceutical compositions of the inventive compounds. Such pharmaceutical compositions may be for administration for injection, or for oral, pulmonary, nasal, transdermal or other forms of administration. In general, the invention encompasses pharmaceutical compositions comprising effective amounts of a compound of the invention together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hyaluronic acid may also be used, and this may have the effect of promoting sustained duration in the circulation. Such compositions may influence the physical state, stability, rate of in vivo release, and rate of in vivo clearance of the present proteins and derivatives. See, e.g. Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder, such as lyophilized form. Implantable sustained release formulations are also contemplated, as are transdermal formulations. These administration alternatives are well known in the art.

The dosage regimen involved in a method for treating the above-described conditions will be determined by the attending physician, considering various factors which modify the action of drugs, e.g. the age, condition, body weight, sex and diet of the patient, the severity of any infection, time of administration and other clinical factors. Generally, the daily regimen should be in the range of 0.1-1000 micrograms of the inventive compound per kilogram of body weight, preferably 0.1-150 micrograms per kilogram.

In some embodiments, the present invention provides a method for the treatment of Aβ-related pathologies such as Downs syndrome and β-amyloid angiopathy, such as but not limited to cerebral amyloid angiopathy, systemic amyloidosis, inclusion body myositis, hereditary cerebral hemorrhage, disorders associated with cognitive impairment, such as but not limited to MC1 (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, comprising administering to a mammal (including human) a therapeutically effective amount of a fusion protein according to the present invention.

### EXAMPLES

### Example 1

### Cloning

A human wt-s neprilysin sequence comprising the codons for aa51-aa749 (PDB numbering) was cloned into a yeast expression vector (pYES2 Invitrogen, SKU#V825-20; see SEQ ID NO:22). Alternative other yeast expression vectors beside pYES2 like pESC-URA (Stratagen; see SEQ ID NO:23) or p427-TEF(Dualsystems Biotech; see SEQ ID NO:24) can be used.

The s neprilysin sequence in the resulting construct is N-terminal fused to sequences encoding a secretion leader, secretion site, triple HA-tag and a dipeptide linker (see SEQ ID NO:5). The triple HA-tag serves for purification of expressed s neprilysin. Alternatively a His-tag can be used. Nucleotide and amino acid sequences of the wt-s neprilysin construct with tag and dipeptide linker are shown in SEQ ID NO: 5 and 3 respectively.

Variants were generated by oligo based site-specific mutagenesis.

3×HA-tag was introduced via 2-step PCR. A first PCR was performed using primer NEP-85A and NEP-24

NEP-85A
(SEQ ID NO: 19)
5′GAC GTC CCA GAC TAT GCT TAc CCt TAc GAt GTa CCt GAt TAc GCa GGA TCC
TAC GAT GAT GGT ATT TGC AAG
NEP-24
(SEQ ID NO: 20)
5′ATA GTT TAG CGG CCG CTC ACC AAA CCC GGC ACT T

A second PCR was performed on the foregoing PCR amplification product using primers NEP-85B and NEP-24, introducing additionally XhoI and NotI restriction endonuclease sites.

NEP-85B
(SEQ ID NO: 21)
5′ GTA TCT CTC GAG AAA AGA GAG GCT GAA GCT TAT CCA TAT GAC GTC CCA
GAC TAT GCT TAT CCA TAT GAC GTC CCA GAC TAT GCT TAC
Underlined sequence is XhoI site.
NEP-24
(SEQ ID NO: 20)
5′ATA GTT TAG CGG CCG CTC ACC AAA CCC GGC ACT T
Underlined sequence is NotI site.

For ligation of PCR amplification product into the expression vector pYES2 containing a secretion leader, the PCR amplification product and the vector were digested with XhoI and NotI with a subsequent ligation reaction using standard molecular biology protocols, resulting in a construct with the nucleotide sequence shown in SEQ ID NO: 7, wherein the alpha secretion leader sequence including the secretion site is at position 507-773, the 3×HA tag sequence is at position 774-854; the Gly/Ser linker (Dipeptid-linker) is at position 855-860; the s neprilysin sequence is at position 861-2960 (wt sequence shown); and the CYY1 terminator sequence is at position 3090-3338.

### Example 2

### Expression and Purification

Expression of mammalian neprilysin in yeast is described in the literature for Schizosaccharomyces pombe and Pichia pasoris (Beaulieu et al. (1999), Oefner et al. (1999)). Using the construct described in Example 1s neprilysin and variants with mutations were expressed in Saccharomyces cerevisiae YMR307w (EUROSCARF) cultured in SC-Media (YB-Yeast, Nitrogen Base (Becton, Dickinson, #291920), CSM-Ura (MPBio, #4511-222), 0.5% casein hydrolysate, 0.2M HEPES (Merck, #1.010110.1000); pH7.0) with 2% galactose (Merck, #1.04061.1000) for induction of expression for 55-70 h at 30° C. (FIG. 4).

Purification of HA-tagged protease can be achieved by immunoaffinity chromatography specific for the HA-tag (monoclonal Antibody HA.11, #MMS-101P) or alternatively for His-tagged protease by metal-chelate affinity chromatography. (Coligan, J. E., Dunn, B. M., Ploegh, H. L., Speicher, D. W., Wingfield, P. T. (Eds.), Current Protocols in Protein Science, John Wiley & Sons, New York (1996) 9.4 and 9.5, respectively). In the latter case pre loading the protease in the yeast supernatant was re-buffered using a cross-filtration device (VIVAFLOW 200, 10k MWCO, Satorius, #512-4069).

Eluted chromatography samples were re-buffered into 50 mM Hepes (sigma, #H4034), 300 mM NaCl (Merck, #1.06404.5000), pH7, by dialysis or the use of desalting columns (Sephadex G-25, Amersham Pharmacia Biotech).

Un-tagged protease can be purified by ion exchange chromatography on resource Q (Amersham Pharmacia Biotech) followed by gel filtration chromatography on Superdex 200 (Amersham Pharmacia Biotech) (Coligan, J. E., Dunn, B. M., Ploegh, H. L., Speicher, D. W., Wingfield, P. T. (Eds.), Current Protocols in Protein Science, John Wiley & Sons, New York (1999) 8.2 and (1998) 8.3, respectively).

### Example 3

### Determination of Catalytic Activity and Specificity

The k_{cat}/k_{M} ratio of a proteolytic activity is proportional to the apparent kinetic constant kₐₚₚ of the determined substrate degradation and is proportional to kcat/Km*[E] ([E]=enzyme concentration). As all measurements are performed at the same enzyme concentration [E], tus the specificity as defines is independent of [E] eliminates from the calculation of relative kcat/Km ratios. This kₐₚₚ was measured as kinetic changes in fluorescence anisotropy for every single substrate. All substrates were customized (Thermo Fisher Scientific GmbH) and were labelled with a fluorophore and a biotin at the N- and C-termini, respectively. The biotin serves to increase the molecular size of uncleaved molecules after addition of streptavidin, thereby increasing the assay window and the measurable signals.

TABLE 4

| Substrate | Label | Amino acid sequence (SEQ ID NO:) | Derivative of |
|---|---|---|---|
| Peptide-1 | Dy647 | DAEFRHDSGYEVHHQKLVFFAEDVGSNKGAI | Aβ1-40 |
| | | IGLMVGGVVK (SEQ ID NO: 8) | |
| Peptide-2 | Dy647 | DAEFRHDSGYEVHHQKLVFFAEDVGSNKGAI | Aβ1-42 |
| | | IGLMVGGVVIAK (SEQ ID NO: 9) | |
| Peptide-3 | Dy505 | SLRRSSCFGGRMDRIGAQSGLGCNSFRYK | ANP |
| | | (SEQ ID NO: 10) | |
| Peptide-4 | Dy505 | SPKMVQGSGCFGRKMDRISSSSGLGCKVLRR | BNP |
| | | HK (SEQ ID NO: 11) | |
| Peptide-5 | Dy505 | CDRVYIHPFHLK (SEQ ID NO: 12) | Angiotensin |
| Peptide-6 | Dy505 | a) GCSSSSLMDKESVYFCHLDIIWK | Endothelin |
| | | (SEQ ID NO: 13) | |
| | | or | |
| | | b) GSSCSSLMDKECVYFSHLDIIWK | |
| | | (SEQ ID NO: 14) | |
| Peptide-7 | Dy505 | CYPSKPDNPGEDAPAEDMARYYSALRHYINL | Neuropeptide Y |
| | | ITRQRYK (SEQ ID NO: 15) | |
| Peptide-8 | Dy505 | CQLYENKPRRPYILK (SEQ ID NO: 16) | Neurotensin |
| Peptide-10 | Dy505 | FVNQHLCGSHLVEALYLVCGERGFFYTPKTK | Ins-B-chain |
| | | (SEQ ID NO: 17) | |
| Peptide-13 | Dy505 | CRPPGFSPFRK (SEQ ID NO: 18) | Bradykinin |
| | or | | |
| | Dy647 | | |

The assay was performed by incubating the protease sample in a microtitre plate with an assay solution composed of 60 nM peptide substrate in 50 mM Hepes (sigma, #H4034), 150 mM NaCl (Merck, #1.06404.5000) and 0.05% PluronicF68 (Sigma, #P7061-500), pH7.0. After incubation of this assay at 37° C. suitable for dynamic measurements (turnover of 5 to 90% of the substrate molecules) the assay was stopped by diluting the sample with an equal volume of 1.2 μM Streptavidin (Calbiochem, #D36271), in the case of assays with peptide-3 or peptide-6 this solution contained 10 mM DTT (Sigma, #117K0663) in addition. A typical incubation time for peptide-1 and -2 was 21 h, for peptide-4 and -7 24 h, for peptide-10 6 h, for peptide-3 and -6 2.5 h and digests of peptide-5, -8, -13 were incubated for 40 min. The anisotropy in the sample was measured in a MTP-reader with an appropriate setup of polarisation filters (Tecan infinite F500; filters: 485/20, 535/25, 625/35, 670/25). Peptides 1-6a,6b, 7, 8, 10 and 13 correspond to SEQ ID NO: 8-18, respectively.

Table 3 depicts the specific activities of a variety of mutants against each of the peptides substrates shown in Table 4.

### Example 4

### Multiple Substitution Mutants

The specific activities against the various peptides that each of the mutants exhibited (Table 3) identified certain locations and particular substitutions as conferring enhanced activity on amyloid beta and reduced activity on the off-peptides. One of the most effective individual substitutions (in terms in increased activity on Aβ from the first set of experiments was found to be G714K, however other mutants exhibit a stronger decrease in activity on certain of the off-target peptides. It was postulated that combining the best individual substitutions might generate mutants with even greater activity on Aβ and less activity on the off-target-peptides. Accordingly, variants with a combination of mutations were generated (Table 5).

In Table 5, the G714K substitution (the single mutation in B9) is included in all clones, B1 to B12. Table 6 lists relative activities of the protease variants vs. mutant G714K on different substrates determined as ratio of the two corresponding kₐₚₚ-values. B1 to B8 (most of them have the mutation G399V), exhibiting a particularly desirable profile of cleavage against the various peptides (in terms of an improved specificity for AB vs. the off-peptides, such as peptide-5, -8, -13, -3, -6 and -10; see Table 6).

A particular embodiment, the G399V/G714K double mutant, shows an improved specificity for AB vs. peptide-5, -8, -13 and -3 by a factor of >100; vs. peptide-4 by a factor of ˜50; and, vs. peptide-6, -10 and -7 by a factor of >10.

TABLE 5
Mutants:

| | CLONE |
|---|---|
| Substitutions | NOMENCLATURE |
| G399V/G714K | B1 |
| S101I/G399V/G714K | B2 |
| S100I/S101Y/G399V/G714K | B3 |
| D107V/G399V/G714K | B4 |
| S100I/S101I/D107V/N403D/W693C/G714K | B5 |
| D107N/G399V/G714K | B6 |
| R102P/G104W/G399V/W693N/G714K | B7 |
| G399W/W693F/G714K | B8 |
| G714K | B9 |
| D107N/Q122R/W693F/G714K | B10 |
| D107V/R292Q/G399V/W693N/G714K | B11 |
| W693L/G714K | B12 |

TABLE 6
activity data

| | Peptide-1 | | | | | | | | |
|---|---|---|---|---|---|---|---|---|---|
| | fold | Peptide-5 | Peptide-8 | Peptide-13 | Peptide-3 | Peptide-6 | Peptide-10 | Peptide-7 | Peptide-4 |
| | G714K | fold | fold | fold | fold | fold | fold | fold | fold |
| | (=clone | G714K | G714K | G714K | G714K | G714K | G714K | G714K | G714K |
| CLONE | B9) act. | act. | act. | act. | act. | act. | act. | act. | act. |
| B1 | 0.79 | 0.02 | 0.01 | 0.03 | 0.02 | 0.14 | 0.44 | 0.82 | 1.00 |
| B2 | 0.69 | 0.02 | 0.01 | 0.03 | 0.02 | 0.11 | 0.29 | 1.55 | 2.50 |
| B3 | 0.55 | 0.01 | 0.01 | 0.03 | 0.02 | 0.05 | 0.16 | 1.35 | 1.00 |
| B4 | 0.54 | 0.01 | 0.01 | 0.03 | 0.02 | 0.10 | 0.33 | 1.28 | 1.00 |
| B5 | 0.45 | 0.01 | 0.01 | 0.03 | 0.02 | 0.09 | 0.35 | 0.70 | 1.00 |
| B6 | 0.39 | 0.01 | 0.01 | 0.03 | 0.02 | 0.09 | 0.31 | 0.75 | 1.00 |
| B7 | 0.41 | 0.01 | 0.01 | 0.03 | 0.02 | 0.06 | 0.36 | 0.77 | 1.00 |
| B8 | 1.52 | 0.57 | 0.14 | 0.42 | 1.05 | 0.66 | 1.28 | 9.39 | 15.44 |
| B9 | 1.00 | 1.00 | 1.00 | 1.00 | 1.00 | 1.00 | 1.00 | 1.00 | 1.00 |
| B10 | 0.64 | 0.50 | 0.97 | 5.30 | 1.17 | 0.39 | 1.29 | 2.64 | 1.00 |
| B11 | 1.28 | 1.30 | 2.67 | 12.95 | 2.65 | 1.01 | 1.84 | 8.28 | 4.31 |
| B12 | 0.55 | 0.57 | 1.11 | 6.76 | 1.62 | 0.45 | 1.21 | 4.61 | 1.00 |

On the basis of B1 (G399V/G714K double mutant) further variants with a combination of additional substitutions were generated (Table 7). Table 8 lists relative activities of the certain protease variants vs. B1 on different substrates determined as ratio of the two corresponding kₐₚₚ-values. C1 to C23 exhibit an increased activity on Peptide-1 and -2, apart from C2 and C3, and a reduced activity on peptide-6, -5 and -3. Hence all show an improved specificity for peptide-1 and -2 vs. peptide-6, -5 and -3 compared to B1. The differences in activities on peptide-7, -4, -13 and -8 between the variants are not significant in many cases, but they all are lying in the range of the respective activities of B1, hence the specificity of these variants for peptide-1 and -2 vs. peptide-7, -4, -13 and -8 is improved compared to B1.

TABLE 7
Sequences of variants

| | | | | | | | | | | | | | No. of |
|---|---|---|---|---|---|---|---|---|---|---|---|---|---|
| CLONE | 227 | 228 | 247 | 399 | 419 | 590 | 593 | 596 | 600 | 709 | 714 | 718 | Mutations |
| wt | S | R | F | G | E | D | G | F | G | D | G | I | 0 |
| B1 | | | | V | | | | | | | K | | 2 |
| C1 | | | | V | M | | | | | V | K | | 4 |
| C2 | R | G | L | V | M | F | | P | D | | K | | 9 |
| C3 | R | G | L | V | | F | | P | D | | K | | 8 |
| C4 | R | G | | V | M | F | V | | V | | K | | 8 |
| C5 | R | G | | V | M | F | | P | D | | K | | 8 |
| C6 | R | G | | V | M | M | V | | V | | K | | 8 |
| C7 | R | G | | V | | F | V | | L | | K | | 7 |
| C8 | R | G | | V | | F | | | W | | K | | 6 |
| C9 | R | G | | V | | M | V | | L | | K | | 7 |
| C10 | R | | L | V | M | M | V | P | D | | K | | 9 |
| C11 | R | | L | V | M | M | | P | W | | K | | 8 |
| C12 | R | | L | V | | M | | | V | | K | | 6 |
| C13 | | G | L | V | M | M | | | V | | K | | 7 |
| C14 | | G | L | V | M | M | | | D | | K | | 7 |
| C15 | | G | | V | M | M | V | | D | | K | | 7 |
| C16 | | G | | V | M | M | V | P | W | | K | | 8 |
| C17 | | G | | V | M | M | V | P | | | K | | 7 |
| C18 | | G | | V | M | M | V | | W | | K | | 7 |
| C19 | | G | | V | M | M | | P | L | | K | | 7 |
| C20 | | G | | V | | F | V | | L | | K | | 6 |
| C21 | | | L | V | M | M | | | V | | K | | 6 |
| C22 | | | | V | M | F | | P | D | | K | | 6 |
| C23 | | | | V | | F | | P | D | | K | | 5 |

Variants with particularly interesting profiles are shown in Table 8.

TABLE 8

| | Peptide-1 | | | | | | | | | |
|---|---|---|---|---|---|---|---|---|---|---|
| | fold | Peptide-2 | Peptide-6a | Peptide-6b | Peptide-5 | Peptide-3 | Peptide-7 | Peptide-4 | Peptide-13 | Peptide-8 |
| | G399V/ | fold | fold | fold | fold | fold | fold | fold | fold | fold |
| | G714K | G399V/ | G399V/ | G399V/ | G399V/ | G399V/ | G399V/ | G399V/ | G399V/ | G399V/ |
| | (=B1) | G714K | G714K | G714K | G714K | G714K | G714K | G714K | G714K | G714K |
| CLONE | activity | activity | activity | activity | activity | activity | activity | activity | activity | activity |
| B1 | 1.00 | 1.00 | 1.00 | 1.00 | 1.00 | 1.00 | 1.00 | 1.00 | 1.00 | 1.00 |
| C1 | 1.28 | 1.23 | 0.97 | 0.76 | 1.02 | 0.36 | 0.90 | 0.51 | 0.52 | 0.62 |
| C2 | 1.06 | 0.88 | 0.14 | 0.10 | 0.19 | 0.24 | 0.47 | 0.72 | 0.68 | 0.34 |
| C3 | 0.86 | 0.81 | 0.18 | 0.09 | 0.15 | 0.24 | 0.64 | 0.79 | 0.85 | 0.37 |
| C4 | 2.41 | 2.43 | 0.37 | 0.29 | 0.27 | 0.24 | 0.44 | 0.44 | 0.62 | 0.40 |
| C5 | 1.95 | 1.92 | 0.23 | 0.18 | 0.22 | 0.24 | 0.72 | 0.74 | 0.63 | 0.41 |
| C6 | 2.35 | 2.36 | 0.28 | 0.19 | 0.29 | 0.24 | 0.61 | 1.26 | 0.85 | 0.37 |
| C7 | 2.77 | 2.83 | 0.42 | 0.28 | 0.34 | 0.24 | 0.47 | 0.96 | 0.43 | 0.46 |
| C8 | 1.86 | 1.64 | 0.32 | 0.24 | 0.27 | 0.24 | 0.69 | 1.17 | 0.43 | 0.36 |
| C9 | 2.33 | 2.44 | 0.31 | 0.21 | 0.22 | 0.24 | 0.52 | 0.67 | 0.52 | 0.46 |
| C10 | 2.35 | 2.60 | 0.27 | 0.19 | 0.17 | 0.24 | 0.49 | 0.80 | 0.68 | 0.34 |
| C11 | 2.19 | 2.14 | 0.28 | 0.11 | 0.19 | 0.24 | 0.66 | 0.49 | 0.61 | 0.35 |
| C12 | 2.11 | 2.04 | 0.32 | 0.18 | 0.26 | 0.24 | 0.84 | 0.79 | 0.83 | 0.43 |
| C13 | 1.78 | 1.56 | 0.30 | 0.18 | 0.18 | 0.24 | 0.36 | 0.91 | 0.39 | 0.35 |
| C14 | 1.98 | 2.01 | 0.30 | 0.24 | 0.20 | 0.24 | 0.59 | 0.72 | 0.71 | 0.43 |
| C15 | 2.52 | 2.72 | 0.46 | 0.24 | 0.25 | 0.24 | 0.75 | 0.70 | 0.44 | 0.52 |
| C16 | 2.24 | 2.19 | 0.35 | 0.23 | 0.21 | 0.24 | 0.70 | 0.70 | 0.44 | 0.40 |
| C17 | 3.28 | 3.13 | 0.47 | 0.33 | 0.27 | 0.24 | 0.57 | 0.70 | 0.49 | 0.33 |
| C18 | 2.56 | 2.29 | 0.36 | 0.26 | 0.16 | 0.24 | 0.67 | 0.59 | 0.50 | 0.73 |
| C19 | 2.33 | 2.34 | 0.29 | 0.19 | 0.15 | 0.24 | 0.34 | 0.67 | 0.33 | 0.29 |
| C20 | 2.18 | 2.25 | 0.37 | 0.26 | 0.43 | 0.24 | 0.72 | 1.21 | 0.32 | 0.41 |
| C21 | 2.30 | 2.76 | 0.41 | 0.24 | 0.22 | 0.24 | 0.50 | 0.65 | 0.74 | 0.49 |
| C22 | 2.85 | 2.72 | 0.40 | 0.28 | 0.18 | 0.24 | 0.76 | 0.98 | 0.70 | 0.43 |
| C23 | 2.39 | 2.42 | 0.41 | 0.23 | 0.32 | 0.34 | 0.75 | 1.15 | 0.45 | 0.55 |
| mean | 19% | 24% | 18% | 18% | 54% | 180% | 35% | 90% | 79% | 61% |
| error | | | | | | | | | | |

FIG. 5 also illustrates the cleavage of five of the peptide substrates (peptide 5=angiotensin; peptide 3=ANP; peptide 6a=one of the endothelin peptides; peptide 1=AB₁₋₄₀; and, peptide 2=AB₁₋₄₂) by various mutants relative to the G399V/G714K parent mutant, illustrating the increased cleavage of the amyloid beta peptides (AB₁₋₄₀ and AB₁₋₄₂) and reduced cleavage of the three off-peptides (ANP, endothelin and angiotensin).

Two of these mutants (C22 and C10) were selected as parent molecules and further mutants with one or more of D377G, A287S and G645Q were introduced therein.

TABLE 9

| Position | 227 | 247 | 287 | 377 | 399 | 419 | 590 | 593 | 596 | 600 | 645 | 709 | 714 |
|---|---|---|---|---|---|---|---|---|---|---|---|---|---|
| WT | S | F | A | D | G | E | D | G | F | G | G | D | G |
| B1 | | | | | V | | | | | | | | K |
| C1 | | | | | V | | | | | | | V | K |
| C22 | | | | | V | M | F | | P | D | | | K |
| D1 | | | S | | V | M | F | | P | D | | | K |
| D2 | | | | G | V | M | F | | P | D | | | K |
| D3 | | | | | V | M | F | | P | D | Q | | K |
| D4 | | | S | G | V | M | F | | P | D | | | K |
| D5 | | | S | G | V | M | F | | P | D | Q | | K |
| C10 | R | L | | | V | M | M | V | P | D | | | K |
| D6 | R | L | S | | V | M | M | V | P | D | | | K |
| D7 | R | L | | G | V | M | M | V | P | D | | | K |
| D8 | R | L | | | V | M | M | V | P | D | Q | | K |
| D9 | R | L | S | G | V | M | M | V | P | D | | | K |
| D10 | R | L | S | G | V | M | M | V | P | D | Q | | K |

Specificity data are shown in Table 10.

| | Pep- | Pep- | Pep- | Pep- | Pep- | Pep- |
|---|---|---|---|---|---|---|
| | tide-1 | tide-2 | tide-6a | tide-5 | tide-7 | tide-4 |
| | fold | fold | fold | fold | fold | fold |
| | G399V/ | G399V/ | G399V/ | G399V/ | G399V/ | G399V/ |
| | G714K (=B1) | G714K | G714K | G714K | G714K | G714K |
| clone | activity | activity | activity | activity | activity | activity |
| B1 | 1.00 | 1.00 | 1.00 | 1.00 | 1.00 | 1.00 |
| C1 | 1.14 | 1.12 | 0.76 | 0.86 | 0.96 | 1.10 |
| C22 | 2.29 | 2.27 | 0.30 | 0.08 | 0.57 | 1.07 |
| D1 | 1.11 | 1.24 | 0.12 | 0.08 | 0.61 | 0.95 |
| D2 | 0.83 | 1.06 | 0.08 | 0.08 | 0.40 | 0.90 |
| D3 | 3.11 | 3.42 | 0.47 | 0.10 | 0.95 | 1.04 |
| D4 | 2.43 | 2.71 | 0.28 | 0.08 | 0.51 | 0.91 |
| D5 | 2.94 | 3.37 | 0.35 | 0.08 | 0.90 | 0.90 |
| C10 | 2.05 | 2.39 | 0.28 | 0.08 | 0.68 | 0.83 |
| D6 | 2.24 | 2.37 | 0.35 | 0.08 | 0.68 | 0.89 |
| D7 | 0.88 | 1.18 | 0.13 | 0.09 | 0.66 | 0.91 |
| D8 | 2.58 | 2.66 | 0.44 | 0.09 | 0.82 | 0.95 |
| D9 | 1.07 | 1.11 | 0.06 | 0.08 | 0.43 | 0.86 |
| D10 | 1.33 | 1.26 | 0.08 | 0.08 | 0.33 | 0.84 |

The data for representative clones in Table 10 is illustrated in FIG. 6.

### Example 5

### Construction of the Gene Encoding the Fusion Protein Fc-Neprilysin Variant, Its Expression and Purification

### A. Construction of Fc-Neprilysin Variant Expression System

The extra-cellular domain of a variant neprilysin containing one or more mutations that impact the specificity of the protease for one or more of its substrates, is fused to the human IgG1 Fc domain (including the hinge region). A signal sequence—MGWSCIILFLVATATGAHS (SEQ ID NO: 25) is introduced to enable secretion of the protein into the culture media during expression. The sequence of the hinge region is THTCPPCP (SEQ ID NO: 26) and the IgG1 Fc domain is shown in SEQ ID NO: 27. The complete fusion protein (excluding the signal sequence) with a human neprilysin variant has predicted molecular weights of 211 kDa (Fc-Nep as a dimer).

The complete gene (encoding the Fc-Neprilysin variant) including the signal sequence is inserted into a suitable mammalian expression vector, such as pCEP4, pEAK10, pEFS/FRT/V5-DEST and pcDNAS/FRT/TO (Gateway adapted). All these are standard mammalian expression vectors based on a CMV promoter (pCEP4, pEAK10 and pcDNAS/FRT/TO) or EF-1a promotor (pEFS/FRT/V5-DEST). After all cloning steps, it is advisable to sequence the genes to verify that the correct sequence exists in the vector.

### B. Expression of Extra-Cellular Domain of Nep and Fusion Protein Fc-Nep in HEK293 Cells

The protein NEP (extra-cellular domain only) and Fc-NEP (Fc-Nep) are transiently expressed in suspension-adapted mammalian cells. The cell lines used in the production experiments may be cell lines derived from HEK293, including HEK293S, HEK293S-T and HEK293S-EBNA cells. Transfection is performed at cell density of approximately 0.5−1×10⁶ and with plasmid DNA at concentrations ranging from 0.3-0.8 μg/ml cell suspension (final concentration). Expression is performed in cell culture volumes of 30 ml to 1000 ml (shaker flasks), and 5L to 10L Wave Bioreactor. Cell cultures are harvested after 4 to 14 days by centrifugation.

### C. Purification of Expressed Fc-Neprilysin Protein by Affinity Chromatography

Purification of the fusion protein can be performed using cell media from expression in mammalian cells. The purification can be performed by Affinity chromatography (Protein A) followed by low pH elution, on ÄKTA Chromatography systems (Explorer or Purifier, GE Healthcare). rProtein A Sepharose FF (GE Healthcare) in an XK26 column (GE Healthcare) is equilibrated with 10 column volumes (CV) of PBS (2.7 mM KCl, 138 mM NaCl, 1.5 mM KH₂PO₄, 8 mM Na₂HPO₄-7H₂O, pH 6.7-7.0, Invitrogen). Cell culture media with expressed fusion protein (Fc-Neprilysin) is applied onto the column. The column is washed with 20 CV PBS before bound protein is eluted with Elution buffer (0.1 M Glycine, pH 3.0). Purified fractions are immediately neutralized by adding 50 μl of 1M Tris Base to 1 ml of eluted protein. Purified fractions are pooled and buffer is exchanged to 50 mM Tris-HCl, pH 7.5, 150 mM NaCl using PD10 Columns (GE Healthcare).

### Example 6

### Degradation of Amyloid β Peptide1-40 in Human Plasma by Neprilysin or Neprilysin Variants

Degradation of human amyloid β peptide1-40 (Aβ40) and human amyloid β peptide1-42 (Aβ42) by Neprilysin is investigated using heparinised plasma from healthy volunteer humans. Human heparin plasma is prepared by centrifugation for 20 min at 4° C. at 2500×g within 30 minutes of sampling. Plasma samples are transferred to pre-chilled polypropylene tubes and immediately frozen and stored at −70° C. prior to use. Neprilysin or Neprilysin variants (0.1-300 mg/ml) or 5 mg/ml recombinant human Neprilysin (R&D systems) with corresponding vehicles (50 mM Tris-HCl, 150 mM NaCl pH 7.5 or 25 mM Tris-HCl, 0.1 M NaCl pH 8.0 or 50 mM HEPES, 100 mM NaCl, 0.05% BSA pH 7.4) are incubated with a pool of plasma in presence or absence of 10 μM phosphoramidon (BIOMOL) or 2 mM 1,10-phenantroline (Sigma-Aldrich) at room temperature for 0, 1 h and 4 h. A final concentration of 5 mM EDTA is added to the tubes before the amount of Aβ40 and Aβ42 is analysed using a commercial ELISA kit obtained from Biosource/Invitrogen (Aβ1-40) or Innogenetics (Aβ1-42).

### Example 7

### Degradation of Amyloid β Peptide1-40 in C57BL/6 Mice by Neprilysin or Neprilysin Variants (In Vivo Studies)

In vivo studies in C57BL/6 mice are performed in order to test the in vivo efficacy of neprilysin or neprilysin variants. The read-outs are soluble amyloid beta (Aβ) levels in plasma as well as plasma drug concentration. The C57BL/6 mice, 17-21 g, are weighed and given single intravenous administration of appropriate doses. 5 animals are included in each time point and each time point has its own vehicle group. Blood is withdrawn from anaesthetized mice by heart puncture into pre-chilled microtainer tubes containing EDTA. Blood samples are immediately put on ice prior to centrifugation. Plasma is prepared by centrifugation for 10 minutes at approximately 3000×g at +4° C. Aβ40 levels in plasma are analyzed by commercial ELISA kit obtained from Biosource. All plasma samples are analysed to determine drug exposure with Mesoscale technology.

### Example 8

### Degradation of Mouse Amyloid β Peptide1-40 in Mouse C57BL/6 Plasma by Neprilysin or Neprilysin Variants

Degradation of mouse amyloid β peptide1-40 (Aβ40) by neprilysin is investigated using heparinized plasma from male and female C57BL/6 mice (20-30 g). Blood is withdrawn from anaesthetized mice by heart puncture. The blood is collected into prechilled microtainer tubes containing heparin and centrifuged for 10 min at 4° C. at 3000×g within 20 minutes of sampling. Plasma samples are transferred to pre-chilled polypropylene tubes and immediately frozen on dry ice and stored at −70° C. prior to use. The experiments are performed on a pool of plasma. neprilysin or neprilysin variants (0.1-300 μg/ml) or 5 μg/ml recombinant human neprilysin (R&D systems) with corresponding vehicles (50 mM Tris-HCl, 150 mM NaCl pH 7.5 or 25 mM Tris-HCl, 0.1 M NaCl pH 8.0 or 50 mM HEPES, 100 mM NaCl, 0.05% BSA pH 7.4) are incubated with a pool of plasma in presence or absence of 10 μM phosphoramidon (BIOMOL) or 2 mM 1,10-phenantroline (Sigma-Aldrich) at room temperature for 0, 1 h and 4 h. A final concentration of 5 mM EDTA is added to the tubes before the amount of mouse A340 is analysed using a commercial ELISA kit obtained from Biosource (Aβ1-40).

### Example 9

### Treatment of APP_{SWE}-Transgenic Mice with Neprilysin or Neprilysin Variants and Subsequent Analysis on Aβ Levels in Plasma and CNS

In vivo studies in APP_{SWE}-transgenic (Tg2576) mice are performed in order to test the in vivo efficacy of neprilysin or neprilysin variants. The primary read-outs are amyloid beta (Aβ) levels in plasma and CNS as well as plasma drug concentration. The Tg2576 mice, 20-25 g, are weighed and administrated intravenously (i.v.) or intraperitoneally (i.p.) with a single or repeated administration.

Single administration of appropriate doses are given to transgenic mice (25-27 weeks of age), including 5-6 animals for each group. Each time point has its own vehicle group. Blood is withdrawn from anaesthetized mice by heart puncture into pre-chilled microtainer tubes containing EDTA. Blood samples are immediately put on ice prior to centrifugation. Plasma is prepared by centrifugation for 10 minutes at approximately 3000×g at +4° C. After blood sampling, mice are sacrificed by decapitation and brain samples are collected. One brain hemisphere is homogenized with 0.2% diethylamine (DEA) and 50 mM NaCl (18 μl/mg tissue). Brain homogenates are centrifuged at 133,000×g for 1 hour at +4° C. Recovered supernatants are neutralised to pH 8.0 with 2 M Tris-HCl. Aβ40 and Aβ42 levels in plasma and brain are analyzed by commercial ELISA kit obtained from Biosource or Innogenetics, respectively. All plasma samples are analysed to determine drug exposure with mesoscale technology.

Repeated administration of appropriate doses are given to transgenic mice (25-27 weeks of age at study start), including 30 animals for each group. Each time point has its own vehicle group. During the time of the study, blood is withdrawn from mice every second week into pre-chilled microtainer tubes containing EDTA. Blood samples are immediately put on ice prior to centrifugation. Plasma is prepared by centrifugation for 10 minutes at approximately 3000×g at +4° C. Drug concentration and immunogenicity are measured in the plasma during the study period with mesoscale technology. At termination, blood is withdrawn from anaesthetized mice by heart puncture into pre-chilled microtainer tubes containing EDTA and plasma is prepared as described above. CSF is aspirated from the cisterna magna and transferred to pre-chilled eppendorf tubes prior to centrifugation. CSF is centrifuged for 1 minute at approximately 3000 g at +4° C. The supernatant is collected and put in new pre-chilled eppendorf tubes. The tubes are immediately frozen on dry ice and stored frozen at −70° C. After sampling, mice are sacrificed by decapitation and brain samples are collected. One brain hemisphere is homogenized with 0.2% diethylamine (DEA) and 50 mM NaCl (18 μl/mg tissue). Brain homogenates are centrifuged at 133,000×g for 1 hour at +4° C. Recovered supernatants are neutralised to pH 8.0 with 2 M Tris-HCl. The insoluble pellet is further sonicated with 70% formic acid (FA) (18 μl/mg tissue). Brain homogenates are centrifuged at 133,000×g for 1 hour at +4° C. Recovered supernatants are neutralised to pH 8.0 with 1 M Tris. Aβ40 and Aβ42 levels in plasma, brain and CSF are analyzed by commercial ELISA kit obtained from Biosource or Innogenetics, respectively. All plasma samples are analysed to determine drug exposure.

### Example 10

### Degradation of Amyloid β Mouse Peptide1-40, Amyloid β Human Peptide1-40 and Amyloid β Human Peptide1-42 in Tg2576 Mouse Plasma by Neprilysin or Neprilysin Variants

Degradation of mouse amyloid β peptide1-40 (Aβ40), human amyloid β peptide1-40 (Aβ40) and human amyloid β peptide1-42 (Aβ42) by neprilysin is investigated using heparinised plasma from female Tg2576 mice (20-30 g). Blood is withdrawn from anaesthetized mice by heart puncture. The blood is collected into prechilled microtainer tubes containing heparin and centrifuged for 10 min at 4° C. at 3000×g within 20 minutes of sampling. Plasma samples are transferred to pre-chilled polypropylene tubes and immediately frozen on dry ice and stored at −70° C. prior to use. The experiments are performed on a pool of plasma. neprilysin or neprilysin variants (0.1-300 μg/ml) or 5 μg/ml recombinant human Neprilysin (R&D systems) with corresponding vehicles (50 mM Tris-HCl, 150 mM NaCl pH 7.5 or 25 mM Tris-HCl, 0.1 M NaCl pH 8.0 or 50 mM HEPES, 100 mM NaCl, 0.05% BSA pH 7.4) are incubated with a pool of plasma in presence or absence of 10 μM phosphoramidon (BIOMOL) or 2 mM 1,10-phenantroline (Sigma-Aldrich) at room temperature for 0, 1 h and 4 h. A final concentration of 5 mM EDTA is added to the tubes before the amount of Aβ40 and Aβ42 is analysed using a commercial ELISA kit obtained from Biosource/Invitrogen (Aβ1-40) or Innogenetics (Aβ1-42).

### Example 11

### Degradation of Amyloid β Peptides in Sprague Dawley Rats by Neprilysin or Neprilysin Variants (In Vivo Studies)

In vivo studies in male Sprague Dawley (SD) rats are performed in order to test the in vivo efficacy of neprilysin or neprilysin variants. The read-outs are soluble amyloid beta (Aβ) levels in plasma, csf and brain as well as plasma drug concentration. The male SD rats (250-350 g) are weighed and given single or repeated intravenous administration of appropriate doses. 8-10 animals are included in each time point and each time point has its own vehicle group. Blood is withdrawn from anaesthetized rats by heart puncture into pre-chilled microtainer tubes containing EDTA. Blood samples are immediately put on ice prior to centrifugation. Plasma is prepared by centrifugation for 10 minutes at approximately 3000×g at +4° C. CSF is aspirated from the cisterna magna and transferred to pre-chilled eppendorf tubes prior to centrifugation. CSF is centrifuged for 1 minute at approximately 3000 g at +4° C. The supernatant is collected and put in new pre-chilled eppendorf tubes. The tubes are immediately frozen on dry ice and stored frozen at −70° C. After sampling, rats are sacrificed by decapitation and brain samples are collected. One brain hemisphere is homogenized with 0.2% diethylamine (DEA) and 50 mM NaCl (18 μl/mg tissue). Brain homogenates are centrifuged at 133,000×g for 1 hour at +4° C. Recovered supernatants are neutralised to pH 8.0 with 2 M Tris-HCl. Soluble Aβ40 in plasma as well as soluble Aβ40 and Aβ42 levels in brain and CSF are analyzed by commercial ELISA kit obtained from Biosource. All plasma samples are analysed to determine drug exposure with Mesoscale technology.

### Example 12

### Degradation of Amyloid β Rat Peptide1-40 in Rat Plasma by Neprilysin or Neprilysin Variants

Degradation of rat amyloid β peptide1-40 (Aβ(40) by Neprilysin is investigated using heparinised plasma from male Sprague Dawley rats (250-350 g). Blood is withdrawn from anaesthetized rats by heart puncture. The blood is collected into prechilled microtainer tubes containing heparin and centrifuged for 10 min at 4° C. at 3000×g within 20 minutes of sampling. Plasma samples are transferred to pre-chilled polypropylene tubes and immediately frozen on dry ice and stored at −70° C. prior to use. The experiments are performed on a pool of plasma. Neprilysin or Neprilysin variants (0.1-300 μg/ml) or 5 μg/ml recombinant human Neprilysin (R&D systems) with corresponding vehicles (50 mM Tris-HCl, 150 mM NaCl pH 7.5 or 25 mM Tris-HCl, 0.1 M NaCl pH 8.0 or 50 mM HEPES, 100 mM NaCl, 0.05% BSA pH 7.4) are incubated with a pool of plasma in presence or absence of 10 μM phosphoramidon (BIOMOL) or 2 mM 1,10-phenantroline (Sigma-Aldrich) at room temperature for 0, 1 h and 4 h. A final concentration of 5 mM EDTA is added to the tubes before the amount of Aβ40 is analysed using a commercial ELISA kit obtained from Biosource/Invitrogen (Aβ1-40).

### Example 13

### Degradation of Amyloid β Peptides in Guinea Pigs by Neprilysin or Neprilysin Variants (In Vivo Studies)

In vivo studies in male Dunkin Hartley (DH) Guinea pigs are performed in order to test the in vivo efficacy of neprilysin or neprilysin variants. The read-outs are soluble amyloid beta (Aβ) levels in plasma, csf and brain as well as plasma drug concentration. The male DH guinea pigs (200-4000 g) are weighed and given single or repeated intravenous administration of appropriate doses. 8-10 animals are included in each time point and each time point has its own vehicle group. CSF is aspirated from the cisterna magna from anaesthetized animals and transferred to pre-chilled eppendorf tubes prior to centrifugation. CSF is centrifuged for 1 minute at approximately 3000 g at +4° C. The supernatant is collected and put in new pre-chilled eppendorf tubes. The tubes are immediately frozen on dry ice and stored frozen at −70° C. Immediately after the CSF sampling, blood is collected by heart puncture into pre-labeled and pre-chilled microtainer tubes containing EDTA. Blood samples are immediately put on ice prior to centrifugation. It is important that the exact sampling times are recorded. Plasma is prepared by centrifugation for 10 minutes at approximately 3000 g at 4° C. within 20 minutes from sampling. After sampling, the animals are sacrificed by decapitation and brain samples are collected. One brain hemisphere is homogenized with 0.2% diethylamine (DEA) and 50 mM NaCl (20 μL/mg wet weight tissue). Brain homogenates are centrifuged at 133,000×g for 1 hour at +4° C. Recovered supernatants are neutralised to pH 8.0 with 2 M Tris-HCl. Soluble Aβ40 and Aβ42 levels in plasma, brain and CSF are analyzed by commercial ELISA kit obtained from Biosource and Innogenetics, respectively. All plasma samples are analysed to determine drug exposure with Mesoscale technology.

### Example 14

### Treatment of APP_{SWE}-Transgenic Mice with Neprilysin or Neprilysin Variants and Subsequent Analysis on Soluble Aβ Levels in Plasma

The objective with this study is to evaluate the time and dose-response effect of neprilysin variants in plasma of female APP_{SWE}-tg mice after acute intravenous treatment. The specific purpose is to find an effect on plasma Aβ₄₀ and Aβ₄₂.

25-31 weeks old female APP_{SWE}-transgenic mice (10 mice/group) receive vehicle or the neprilysin variants at 1 or 5 mg/kg as a single intravenous injections. The animals are treated in 3 hours (4 mice). A blank group is also included in the study. Blood is sampled from vehicle- and compound-treated animals at 1,5 and 3 hours after dose. Blood is withdrawn from anaesthetized mice by heart puncture into pre-chilled microtainer tubes containing EDTA. Blood samples are immediately put on ice prior to centrifugation. Plasma is prepared by centrifugation for 10 minutes at approximately 3000×g at +4° C. within 20 minutes from sampling. After blood sampling, mice are terminated. Aβ40 and Aβ42 levels in plasma are analyzed by commercial ELISA kit obtained from Biosource and Innogenetics, respectively.

### Example 15

### EEG study in APP_{SWE}-Transgenic Mice with Neprilysin or Neprilysin Variants (In Vivo Studies)

The studies in mice can be complemented with a read-out with EEG. Mice are implanted with an indwelling electrode consisting of three polyimide-coated wires with bare tips that are implanted at depths 3 mm, 1 mm, and 1 mm from the dorsal surface of the brain to target the CA3 region of the hippocampus (2.5 mm posterior and 2 0 mm lateral from Bregma) and cortical surfaces (1 and 2 mm rostral from hippocampal wire), respectively. Electrode location is verified in a subset of animals to show proper targeting of the hippocampal area. Data is recorded continuously during the dark (night; active) cycle (6 pm-6 am). Normally data is analysed from the first two hours of the dark cycle separately and presented as representative.

Signals are interpolated to 128 Hz and band-passed filtered 1-64 Hz (second order Butterworth). Power spectral densities (PSDs) are calculated with Fast Fourier Transform (FFT) to convert the waveform data into a power spectrum with 0.5 Hz resolution (FFT size of 256) using Spike2 (Cambridge Electronic Design). PSDs are calculated from the entire recording. Spectrograms are generated and power spectra are calculated for each one second using an FFT of 128 Hz and color-mapped as terms of Log of PSD calculated as 10*log10(raw P SD), where raw PSD is normalized so that the sum of all the spectrum values equals to the mean squared value of the signal. Power scales are globalised and a boxcar filter was used to smooth the resulting spectrogram for visualization. To calculate the dominant frequency (DF) at a specific Hz interval, PSDs are generated as above for every 30 seconds for each individual recording. The DF for each 30 second epoch is the frequency that has the greatest power in that epoch. An average DF is calculated for each mouse from each DF in each 30 second epoch (3600/30 s=120 epochs) in its recording. The average DF represents the average of the DFs from all the mice in each group.

### Example 16

### In Vivo Testing of Protease Variants

### 1. Dementia

### The Object Recognition Task

The object recognition task has been designed to assess the effects of experimental manipulations on the cognitive performance of rodents. A rat is placed in an open field, in which two identical objects are present. The rats inspects both objects during the first trial of the object recognition task. In a second trial, after a retention interval of for example 24 hours, one of the two objects used in the first trial, the ‘familiar’ object, and a novel object are placed in the open field. The inspection time at each of the objects is registered. The basic measures in the OR task is the time spent by a rat exploring the two object the second trial. Good retention is reflected by higher exploration times towards the novel than the ‘familiar’ object.

Administration of the putative cognition enhancer prior to the first trial predominantly allows assessment of the effects on acquisition, and eventually on consolidation processes. Administration of the testing compound after the first trial allows to assess the effects on consolidation processes, whereas administration before the second trial allows to measure effects on retrieval processes.

### The Passive Avoidance Task

The passive avoidance task assesses memory performance in rats and mice. The inhibitory avoidance apparatus consists of a two compartment box with a light compartment and a dark compartment. The two compartments are separated by a guillotine door that can be operated by the experimenter. When the door is open, the illumination in the dark compartment is about 2 lux. The light intensity is usually about 500 lux at the centre of the floor of the light compartment.

Two habituation sessions, one shock session, and a retention session are given, separated by inter session intervals of 24 hours. In the habituation sessions and the retention session the rat is allowed to explore the apparatus for 300 sec. The rat is placed in the light compartment, facing the wall opposite to the guillotine door. After an accommodation period of 15 sec. the guillotine door is opened so that all parts of the apparatus can be visited freely. Rats normally avoid brightly lit areas and will enter the dark compartment within a few seconds.

In the shock session the guillotine door between the compartments is lowered as soon as the rat has entered the dark compartment with its four paws, and a scrambled 0.3-1 mA foot shock is administered for 2 sec. The rat is removed from the apparatus and put back into its home cage. The procedure during the retention session is identical to that of the habituation sessions.

The step through latency, that is the first latency of entering the dark compartment (in sec.) during the retention session is an index of the memory performance of the animal; the longer the latency to enter the dark compartment, the better the retention is. A testing compound in given half an hour before the shock session, together with scopolamine Scopolamine impairs the memory performance during the retention session 24 hours later. If the test compound increases the enter latency compared with the scopolamine treated controls, is likely to possess cognition enhancing potential.

### The Contextual Fear Conditioning Task

Contextual fear conditioning measures aversive memory in rats and mice. An observation box with distinctive contextual features are used (light, texture etc) The box is equipped with a gridded floor and stimulus lights located in each compartment. The chamber is made of transparent Plexiglas and illuminated by a 60-W bulb (including dimmers).

On the day of training and testing the animals are first allowed to habituate to the experimental room for 60 minutes. On the first day of experiment (training trial), the animal is placed in the illuminated chamber where it is left to explore the compartment. After a defined time (180 s) a foot shock (usually 0.7 mA, 2 s duration, constant current) is delivered to the animal's feet. The animal is left in the light chamber for an additional 30 s before being returned to its home cage immediately after the training trial. Behavior is recorded again 24 h later (test trial), in the same manner as described above with the exception that no chock is delivered on the test day and the cut off time is 180 s. The readout used is freezing response (i.e. no movement of the animal) and is used as a measure of memory of the previously aversive event in this context. The boxes are controlled by software from the manufacturer. The animals are videotaped and the freezing response is scored manually afterwards Animals are evenly distributed over doses and time of day. Sometimes, the testing compound is given together with scopolamine Scopolamine impairs the memory performance during the retention session 24 hours later. If the test compound increases the enter latency compared with the scopolamine treated controls, is likely to possess cognition enhancing potential.

### The Morris Water Escape Task

The Morris water escape task measures spatial orientation learning in rodents. It is a test system that has extensively been used to investigate the effects of putative therapeutic on the cognitive functions of rats and mice. The performance of an animal is assessed in a circular water tank with an escape platform that is submerged about 1 cm below the surface of the water. The escape platform is not visible for an animal swimming in the water tank. Abundant extra maze cues are provided by the furniture in the room, e.g. desks, computer equipment.

The animals receive four trials during five daily acquisition sessions. A trial is started by placing an animal into the pool, facing the wall of the tank. Each of four starting positions in the quadrants north, east, south, and west is used once in a series of four trials; their order is randomized. The escape platform is always in the same position. A trial is terminated as soon as the animal had climbs onto the escape platform or when 90 seconds have elapsed, whichever event occurs first. The animal is allowed to stay on the platform for 30 seconds. Then it is taken from the platform and the next trial is started. If an animal did not find the platform within 90 seconds it is put on the platform by the experimenter and is allowed to stay there for 30 seconds. After the fourth trial of the fifth daily session, an additional trial is given as a probe trial: the platform is removed, and the time the animal spends in the four quadrants is measured for 30 or 60 seconds. In the probe trial, all animals start from the same start position, opposite to the quadrant where the escape platform had been positioned during acquisition.

Four different measures are taken to evaluate the performance of an animal during acquisition training escape latency, traveled distance, distance to platform, and swimming speed. The following measures are evaluated for the probe trial: time (s) in quadrants and traveled distance (cm) in the four quadrants. The probe trial provides additional information about how well an animal learned the position of the escape platform. If an animal spends more time and swims a longer distance in the quadrant where the platform had been positioned during the acquisition sessions than in any other quadrant, one concludes that the platform position has been learned well.

In order to assess the effects of putative cognition enhancing protease variants, rats or mice with specific brain lesions which impair cognitive functions, or animals treated with compounds such as scopolamine or MK 801, which interfere with normal learning, or aged animals which suffer from cognitive deficits, are used.

### The T Maze Spontaneous Alternation Task

The T maze spontaneous alternation task assesses the spatial memory performance in mice. The start arm and the two goal arms of the T maze are provided with guillotine doors which can be operated manually by the experimenter. A mouse is put into the start arm at the beginning of training. The guillotine door is closed. In the first trial, the ‘forced trial’, either the left or right goal arm is blocked by lowering the guillotine door. After the mouse has been released from the start arm, it will negotiate the maze, eventually enter the open goal arm, and return to the start position, where it will be confined for 5 seconds, by lowering the guillotine door. Then, the animal can choose freely between the left and right goal arm (all guillotine doors opened) during 14 ‘free choice’ trials. As soon as the mouse has entered one goal arm, the other one is closed. The mouse eventually returns to the start arm and is free to visit whichever go alarm it wants after having been confined to the start arm for 5 seconds. After completion of 14 free choice trials in one session, the animal is removed from the maze. During training, the animal is never handled.

The percent alternations out of 14 trials is calculated. This percentage and the total time needed to complete the first forced trial and the subsequent 14 free choice trials (in s) is analyzed. Cognitive deficits are usually induced by an injection of scopolamine, 30 min before the start of the training session. Scopolamine reduced the percent alternations to chance level, or below. A cognition enhancer, which is always administered before the training session, will at least partially, antagonize the scopolamine induced reduction in the spontaneous alternation rate.

### 2. Neuropathic Pain

Neuropathic pain is induced by different variants of unilateral sciatic nerve injury mainly in rats. The operation is performed under anaesthesia. The first variant of sciatic nerve injury is produced by placing loosely constrictive ligatures around the common sciatic nerve. The second variant is the tight ligation of about the half of the diameter of the common sciatic nerve. In the next variant, a group of models is used in which tight ligations or transections are made of either the L5 and L6 spinal nerves, or the L % spinal nerve only. The fourth variant involves an axotomy of two of the three terminal branches of the sciatic nerve (tibial and common peroneal nerves) leaving the remaining sural nerve intact whereas the last variant comprises the axotomy of only the tibial branch leaving the sural and common nerves uninjured. Control animals are treated with a sham operation.

Postoperatively, the nerve injured animals develop a chronic mechanical allodynia, cold allodynioa, as well as a thermal hyperalgesia. Mechanical allodynia is measured by means of a pressure transducer (electronic von Frey Anesthesiometer, IITC Inc. Life Science Instruments, Woodland Hills, SA, USA; Electronic von Frey System, Somedic Sales AB, Hörby, Sweden). Thermal hyperalgesia is measured by means of a radiant heat source (Plantar Test, Ugo Basile, Comerio, Italy), or by means of a cold plate of 5 to 10° C. where the nocifensive reactions of the affected hind paw are counted as a measure of pain intensity. A further test for cold induced pain is the counting of nocifensive reactions, or duration of nocifensive responses after plantar administration of acetone to the affected hind limb. Chronic pain in general is assessed by registering the circadanian rhythms in activity (Surjo and Arndt, Universität zu Köln, Cologne, Germany), and by scoring differences in gait (foot print patterns; FOOTPRINTS program, Klapdor et al., 1997. A low cost method to analyze footprint patterns. J. Neurosci. Methods 75, 49 54).

Protease variants are tested against sham operated and vehicle treated control groups. Substance application is performed at different time points via different application routes (i.v., i.p., p.o., i.t., i.c.v., s.c., intradermal, transdermal) prior to pain testing.

### 3. In Vivo Testing of Cardiovascular Effects of Protease Variants

### Hemodynamics in Anesthetized Rats

Male Wistar rats weighing 300-350 g (Harlan Winkelmann, Borchen, Germany) are anesthetized with thiopental “Nycomed” (Nycomed, Munich, Germany) 100 mg kg-1i.p.

A tracheotomy is performed, and catheters are inserted into the femoral artery for blood pressure and heart rate measurements (Gould pressure transducer and recorder, model RS 3400) and into the femoral vein for substance administration. The animals are ventilated with room air and their body temperature is controlled. Test protease variants are administered intravenously.

### Hemodynamics in Conscious SHR

Female conscious SHR (Moellegaard/Denmark, 220-290 g) are equipped with implantable radiotelemetry, and a data acquisition system (Data Sciences, St. Paul, Minn., USA), comprising a chronically implantable transducer/transmitter unit equipped with a fluid-filled catheter is used. The transmitter is implanted into the peritoneal cavity, and the sensing catheter is inserted into the descending aorta.

Single administration of test protease variant is performed intravenously. The animals of control groups only receive the vehicle. Before treatment, mean blood pressure and heart rate of treated and untreated control groups are measured.

### Example 17

### Construction of the Gene Encoding the 10Histidine Tag Fused to a Neprilysin Variant, its Expression and Purification

### A. Construction of 10His-Neprilysin Variant Expression System

The extra-cellular domain of a variant Neprilysin containing one or more mutations that impact the specificity of the protease for one or more of its substrates, is fused to an N-terminal 10His Tag. A signal sequence -MGWSCIILFLVATATGAHS (SEQ ID NO 25) is introduced to enable secretion of the protein into the culture media during expression. The complete fusion protein (excluding the signal sequence) with a human Neprilysin variant has a predicted molecular weight of approximately 81 kDa.

The complete gene (encoding the 10His-Neprilysin variant) including the signal sequence is inserted into a suitable mammalian expression vector, such as pDEST12.2, pCEP4, pEAK10, pEFS/FRT/V5-DEST and pcDNAS/FRT/TO (Gateway adapted). All these are standard mammalian expression vectors based on a CMV promoter (pDEST12.2, pCEP4, pEAK10 and pcDNAS/FRT/TO) or EF-1a promoter (pEFS/FRT/V5-DEST). After all cloning steps, it is advisable to sequence the genes to verify that the correct sequence exists in the vector.

### B. Expression of Extra-Cellular Domain of Nep and Fusion Protein 10His-NEP in CHO Cells

The 10His-Neprilysin variant is transiently expressed in suspension-adapted CHO cells. The cell lines used in the production experiments may be cell lines derived from CHO-K1. Transfection is performed at cell density of approximately 0.5−1×10⁶ and with plasmid DNA at a concentration of 1 μg/ml cell suspension (final concentration). Expression is performed in cell culture volumes of 30 ml to 500 ml (shaker flasks), and 5 L to 25 L Wave Bioreactor. Cell cultures are harvested after 4 to 14 days by centrifugation.

### C. Purification of Expressed 10His-Neprilysin Protein by Affinity Chromatography

Purification of the fusion protein can be performed using cell media from expression in mammalian cells. The purification can be performed by immobilized metal ion adsorption chromatography (IMAC) using for example, a HisTrap HP or Ni-Sepharaose on an ÄKTA Chromatography system (Explorer or Purifier, GE Healthcare). The column is equilibrated with 10 column volumes (CV) of 2×PBS (5.4 mM KCl, 276 mM NaCl, 3 mM KH₂PO₄, 16 mM Na₂HPO₄-7H₂O, pH 7.4, Invitrogen). Cell culture media with expressed fusion protein (10His-Neprilysin) is applied onto the column. The column is then washed with 20 CV 2×PBS and 10 CV 2×PBS with 40 mM imidazole before being eluted using an imidazole gradient from 40 to 400 mM imidazole over 10 CV. Fractions containing the 10His-Neprilysin protein are pooled and concentrated and further purified using size exclusion chromatography. This can be performed using a Superdex 200 16/60 column (GE Healthcare) on an ÄKTA Chromatography system (Explorer or Purifier, GE Healthcare). The protein is eluted in 1×PBS 2.7 mM KCl, 138 mM NaCl, 1.5 mM KH₂PO₄, 8 mM Na₂HPO₄-7H₂O, pH 7.4, Invitrogen) and the fractions containing 10His Neprilysin pooled, frozen and stored at −80 C.

### Example 18

### Construction of the Gene Encoding the Fusion Protein HSA-Neprilysin Variant, its Expression and Purification

### A. Construction of HSA-Neprilysin Variant Expression System

The extra-cellular domain of a variant neprilysin containing one or more mutations that impact the specificity of the protease for one or more of its substrates, is fused to the human serum albumin (HSA) with or without its propeptide. A signal sequence—MGWSCIILFLVATATGAHS (SEQ ID NO 25) is introduced to enable secretion of the protein into the culture media during expression. The complete fusion protein (excluding the signal sequence) with a human neprilysin variant has predicted molecular weight of approximately 147 kDa.

The complete gene (encoding the HSA-neprilysin variant) including the signal sequence is inserted into a suitable mammalian expression vector, such as pDEST12.2, pCEP4, pEAK10, pEFS/FRT/V5-DEST and pcDNAS/FRT/TO (Gateway adapted). All these are standard mammalian expression vectors based on a CMV promoter (pDEST12.2, pCEP4, pEAK10 and pcDNAS/FRT/TO) or EF-1a promotor (pEFS/FRT/V5-DEST). After all cloning steps, it is advisable to sequence the genes to verify that the correct sequence exists in the vector.

### B. Expression of Extra-Cellular Domain of NEP and Fusion Protein HSA-NEP in CHO Cells

The protein NEP (extra-cellular domain only) and HSA-NEP are transiently expressed in suspension-adapted CHO cells. The cell lines used in the production experiments may be cell lines derived from CHO-K1. Transfection is performed at cell density of approximately 0.5−1×10⁶ and with plasmid DNA at a concentration of 1 μg/ml cell suspension (final concentration). Expression is performed in cell culture volumes of 30 ml to 500 ml (shaker flasks), and 5L to 25L Wave Bioreactor. Cell cultures are harvested after 4 to 14 days by centrifugation.

### C. Purification of Expressed HSA-Neprilysin Protein by Affinity Chromatography

Purification of the fusion protein can be performed using cell media from expression in mammalian cells. The purification can be performed by affinity chromatography using an anti-HSA Affibody column. The Affibody is coupled to Sulfolink resin (Pierce) via its free cysteine and is equilibrated with 10 column volumes (CV) of Buffer A (50 mM Tris, 250 mM NaCl, pH 8). Cell culture media with expressed fusion protein (HSA-Neprilysin) is applied onto the resin. The column is washed with Buffer A before bound protein is eluted with Buffer B (100 mM Glycine, pH 2.7). Purified fractions are immediately neutralized by adding 1 ml of 1 M Tris, pH 8.5 to 10 ml of eluted protein. Purified fractions are pooled, concentrated and further purified using size exclusion chromatography. This can be performed using a Superdex 200 16/60 column (GE Healthcare) on an ÄKTA Chromatography system (Explorer or Purifier, GE Healthcare). The protein is eluted in 1×PBS 2.7 mM KCl, 138 mM NaCl, 1.5 mM KH₂PO₄, 8 mM Na₂HPO₄-7H₂O, pH 7.4, Invitrogen) and the fractions containing HSA-Neprilysin pooled, frozen and stored at −80 C.

### Example 19

### Kinetic Analysis of Peptide Cleavage by Protease Variants

Kinetic parameters Vₘₐₓ, K_{M}, k_{cat} and k_{cat}/K_{M} for cleavage of peptides by protease variants were determined using a fluorescence polarisation assay that measured cleavage of synthetic peptide substrates labelled at the N- and C-termini with fluorescein and biotin, respectively. The biotin serves for increasing the molecular size of uncleaved molecules after addition of avidin, thereby increasing the assay window and the measurable signals. The peptides substrate are shown in Table 11.

TABLE 11
Synthetic peptide substrates. Peptides were labelled at their N-termini with
fluorescein and their C-termini with Lys-biotin.

| Peptide | Sequence | Supplier |
|---|---|---|
| A-beta 1-40 | DAEFRHDSGYEVHHQKLVFFAEDVGSNKGAIIGLMVGGVV | Bachem |
| Neurotensin | QLYENKPRRPYIL | Alta Bioscience |
| ANP | SLRRSSCFGGRMDRIGAQSGLGCNSFRY | Bachem |
| Endothelin-1a | CSSSSLMDKESVYFCHLDIIW | Alta Bioscience |
| Endothelin-1b | SSCSSLMDKECVYFSHLDIIW | Alta Bioscience |
| GLP-1 | HAEGTFTSDVSSYLEGQAAKEFIAWLVKGRG | Alta Bioscience |
| Angiotensin | DRVYIHPFHL | Alta Bioscience |
| Bradykinin | RPPGFSPFR | Alta Bioscience |
| GIP | YAEGTFISDYSIAMDKIHQQDFVNWLLAQKGKKNDWKHNITQ | Alta Bioscience |
| Somatostatin | SANSNPAMAPRERKAGCKNFFWKTFTSC | Alta Bioscience |
| 1-28 | | |
| Glucagon | HSQGTFTSDYSKYLDSRRAQDFVQWLMNT | Alta Bioscience |

The assay was performed in a 96-well microtitre plate and contained 50 mM HEPES (pH 7.4, Sigma, #H3375), 150 mM NaCl, 0.05% (w/v) BSA (Sigma, #A9576), 1-200 nM peptide substrate and 1-500 nM protease variant. Assays with endothelin 1a, endothelin 1b and ANP contained 2 mM tris(2-carboxyethyl)phosphine (Sigma, #C4706) in addition. Reactions were incubated at 37° C. before being stopped at various time points between 2 and 360 min by transferring 5 μL, aliquots to 245 μL, 50 mM HEPES buffer containing 2 mM 1,10-phenanethroline monohydrate (Sigma, #P9375) and 2 μM avidin (Invitrogen, #A2667). The fluorescence polarisation of the resulting solution was measured on a Victor plate reader and the amount of substrate cleaved was determined with reference to substrate-only controls with and without avidin. Initial rates were obtained by linear regression of the linear regions of time courses. Enzyme velocity was plotted as a function of substrate concentration and the Michaelis-Menten equation was used to fit the data, giving the parameters Vₘₐₓ and K_{M}. k_{cat} was calculated by dividing Vₘₐₓ by the enzyme concentration. Catalytic efficiency on a particular substrate was assessed by the second order rate constant kcat/Km, which was expressed in units of M⁻¹s⁻¹.

Table 12 shows k_{cat}/K_{M} values for wild type neprilysin, the G399V/G714K mutant and the fusion of the G399V/G714K mutant with HSA. The ratios of the G399V/G714K variant and HSA fusion of the mutant kcat/Km values to those of wild type neprilysin are shown in Table 13. Catalytic efficiency on Aβ1-40 was increased by a factor of 4.5 in the G399V/G714K mutant, compared to wild type neprilysin. A similar increase in k_{cat}/K_{M} on Aβ1-40 was observed with the HSA fusion of the G399V/G714K mutant. k_{cat}/K_{M} values for cleavage of bradykinin, neurotensin, somatostatin 1-28, angiotensin and ANP were reduced by factors of 3200, 330, 140, 71 and 11, respectively in the G399V/G714K mutant. Similar reductions in catalytic efficiency on these substrates were observed with the HSA fusion of the mutant. kcat/Km values for cleavage of endothelin-1, GIP, and glucagon were reduced by 2-4-fold in the G399V/G714K mutant compared to wild type neprilysin. Similar reductions in catalytic efficiency on these substrates were observed with the HSA fusion of the mutant.

TABLE 12
k_{cat}/K_{M} values for peptide cleavage by neprilysin variants.

| | k_{cat}/K_{M} (M⁻¹s⁻¹) | | |
|---|---|---|---|
| Peptide | | Nep- | HSA-Nep- |
| derivative | Wild type Nep | G399V/G714K | G399V/G714K |

| | | | |
|---|---|---|---|
| A-beta 1-40 | 1.3 × 10⁴ | 5.9 × 10⁴ | 5.5 × 10⁴ |
| Neurotensin | 6.9 × 10⁵ | 2.0 × 10³ | 2.6 × 10³ |
| ANP | 2.9 × 10⁵ | 2.6 × 10⁴ | 4.2 × 10⁴ |
| Endothelin-1 | 3.0 × 10⁵ | 8.6 × 10⁴ | 6.2 × 10⁴ |
| GLP-1 | 1.4 × 10⁵ | 3.8 × 10⁴ | 3.6 × 10⁴ |
| Angiotensin | 1.2 × 10⁶ | 1.6 × 10⁴ | 1.4 × 10⁴ |
| Bradykinin | 4.9 × 10⁵ | 1.5 × 10² | 8.5 × 10¹ |
| GIP | 4.6 × 10² | 1.6 × 10² | 1.3 × 10² |
| Somatostatin 1-28 | 5.6 × 10⁵ | 4.1 × 10³ | 3.0 × 10³ |
| Glucagon | 2.5 × 10⁵ | 1.0 × 10⁵ | 8.8 × 10⁴ |

The k_{cat}/K_{M} values are averages of data from at least two independent experiments. For endothelin-1, the k_{cat}/K_{M} represents the average of values determined in duplicate for the 1a and 1b isoforms.

TABLE 13
Ratios of mutant and wild type neprilysin k_{cat}/K_{M} on various peptides

Ratio mutant vs wild type

| Peptide | Nep- | HSA-Nep- |
|---|---|---|
| derivative | G399V/G714K | G399V/G714K |
| A-beta 1-40 | 4.5 | 4.2 |
| Neurotensin | 0.003 | 0.004 |
| ANP | 0.088 | 0.15 |
| Endothelin-1 | 0.29 | 0.21 |
| GLP-1 | 0.27 | 0.25 |
| Angiotensin | 0.014 | 0.012 |
| Bradykinin | 0.00031 | 0.00017 |
| GIP | 0.34 | 0.28 |
| Somatostatin 1-28 | 0.0073 | 0.0054 |
| Glucagon | 0.41 | 0.35 |

1. A polypeptide comprising a protease variant of wild type human neprilysin extracellular catalytic domain (SEQ ID NO: 2), said polypeptide having a greater specificity for an Aβ peptide compared to wild type human neprilysin (SEQ ID NO: 1), wherein G399 is replaced by another naturally occurring amino acid and/or G714 is replaced by another naturally occurring amino acid, optionally said naturally occurring amino acid is other than Ala (A).

2. A polypeptide comprising a protease variant according to claim 1, wherein G399 is replaced by Valine (V) and/or G714 is replaced by Lysine (K).

3. A polypeptide comprising a protease variant according to claim 1, wherein G399 is replaced by Valine (V) and G714 is replaced by Lysine (K).

4. A polypeptide according to claim 1 comprising a protease variant of wild-type human neprilysin extracellular catalytic domain as shown in SEQ ID NO: 2, said polypeptide having an altered specificity against Amyloid β₄₀, Amyloid β₄₂, Angiotensin-1 and -2, ANP, BNP, bradykinin, Endothelin-1 and -2, Neuropeptide Y, Neurotensin, Adrenomedullin, Bombesin, BLP, CGRP, Enkephalins, FGF-2, fMLP, GRP, Neurokinin A, Neuromedin C, Oxytocin, PAMP, Substance P or VIP.

5. A polypeptide according to claim 1 comprising a protease variant of wild type human neprilysin extracellular domain of SEQ ID NO: 2 having an altered specificity against Amyloid β₄₀, Amyloid β₄₂, Angiotensin-1 and -2, ANP, BNP, bradykinin, Endothelin-1 and -2, Neuropeptide Y or Neurotensin.

6. A polypeptide according to claim 1 comprising a half-life modulator moiety provided N-terminal to the protease variant, preferably said half-life modulator moiety is selected from an Fc domain and a human serum albumin (HSA) or variant thereof, optionally said half-life modulator moiety and protease variant are joined by a linker.

7. A nucleic acid encoding a polypeptide of claim 1.

8. A vector comprising the nucleic acid of claim 7.

9. A host cell comprising the vector of claim 8.

10. A method for producing a polypeptide according to claim 1, wherein the method comprises the following steps:
a. culturing the host cell of claim 9 under conditions suitable for the expression of the protease variant; and
b. recovering the protease variant from the host cell culture.

11. A pharmaceutical composition comprising a polypeptide of claim 1.

12. A method for treating a human neprilysin substrate related disease, such as an Aβ-related pathology, such as Alzheimer's disease, comprising administering to a patient in need thereof a therapeutically effective dose of a polypeptide comprising a protease variant according to claim 1, whereby a symptom of the human neprilysin substrate related disease is ameliorated.

13. (canceled)

14. A polypeptide with increased specificity for Aβ according to claim 4 or 5 for use to prevent and/or treat an Aβ-related pathology such as Alzheimer's disease.

15. (canceled)


SEQUENCE LISTING (50 sequences)

SEQ ID NO: 1 - protein, 749 aa, Homo sapiens
GKSESQMDITDINTPKPKKKQRWTPLEISLSVLVLLLTIIAVTMIALYATYDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 2 - protein, 699 aa, Homo sapiens
YDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 3 - protein, 728 aa, Artificial Sequence
  /note=“Description of Artificial Sequence Synthetic polypeptide”
YPYDVPDYAYPYDVPDYAYPYDVPDYAGSYDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 4 - DNA, 2100 bp, Homo sapiens
tacgatgatggtatttgcaagtcatcagactgcataaaatcagctgctcgactgatccaaaacatggatgccaccactgagccttgtacagactttttcaaatatgcttgcggaggctggttgaaacgtaatgtcattcccgagaccagctcccgttacggcaactttgacattttaagagatgaactagaagtcgttttgaaagatgtccttcaagaacccaaaactgaagatatagtagcagtgcagaaagcaaaagcattgtacaggtcttgtataaatgaatctgctattgatagcagaggtggagaacctctactcaaactgttaccagacatatatgggtggccagtagcaacagaaaactgggagcaaaaatatggtgcttcttggacagctgaaaaagctattgcacaactgaattctaaatatgggaaaaaagtccttattaatttgtttgttggcactgatgataagaattctgtgaatcatgtaattcatattgaccaacctcgacttggcctcccttctagagattactatgaatgcactggaatctataaagaggcttgtacagcatatgtggattttatgatttctgtggccagattgattcgtcaggaagaaagattgcccatcgatgaaaaccagcttgctttggaaatgaataaagttatggaattggaaaaagaaattgccaatgctacggctaaacctgaagatcgaaatgatccaatgcttctgtataacaagatgacattggcccagatccaaaataacttttcactagagatcaatgggaagccattcagctggttgaatttcacaaatgaaatcatgtcaactgtgaatattagtattacaaatgaggaagatgtggttgtttatgctccagaatatttaaccaaacttaagcccattcttaccaaatattctgccagagatcttcaaaatttaatgtcctggagattcataatggatcttgtaagcagcctcagccgaacctacaaggagtccagaaatgctttccgcaaggccctttatggtacaacctcagaaacagcaacttggagacgttgtgcaaactatgtcaatgggaatatggaaaatgctgtggggaggctttatgtggaagcagcatttgctggagagagtaaacatgtggtcgaggatttgattgcacagatccgagaagtttttattcagactttagatgacctcacttggatggatgccgagacaaaaaagagagctgaagaaaaggccttagcaattaaagaaaggatcggctatcctgatgacattgtttcaaatgataacaaactgaataatgagtacttggagttgaactacaaagaagatgaatacttcgagaacataattcaaaatttgaaattcagccaaagtaaacaactgaagaagctccgagaaaaggtggacaaagatgagtggataagtggagcagctgtagtcaatgcattttactcttcaggaagaaatcagatagtcttcccagccggcattctgcagccccccttctttagtgcccagcagtccaactcattgaactatgggggcatcggcatggtcataggacacgaaatcacccatggcttcgatgacaatggcagaaactttaacaaagatggagacctcgttgactggtggactcaacagtctgcaagtaactttaaggagcaatcccagtgcatggtgtatcagtatggaaacttttcctgggacctggcaggtggacagcaccttaatggaattaatacactgggagaaaacattgctgataatggaggtcttggtcaagcatacagagcctatcagaattatattaaaaagaatggcgaagaaaaattacttcctggacttgacctaaatcacaaacaactatttttcttgaactttgcacaggtgtggtgtggaacctataggccagagtatgcggttaactccattaaaacagatgtgcacagtccaggcaatttcaggattattgggactttgcagaactctgcagagttttcagaagcctttcactgccgcaagaattcatacatgaatccagaaaagaagtgccgggtttggtga

SEQ ID NO: 5 - DNA, 2187 bp, Artificial Sequence
  /note=“Description of Artificial Sequence Synthetic polynucleotide”
tatccatatgacgtcccagactatgcttatccatatgacgtcccagactatgcttacccttacgatgtacctgattacgcaggatcctacgatgatggtatttgcaagtcatcagactgcataaaatcagctgctcgactgatccaaaacatggatgccaccactgagccttgtacagactttttcaaatatgcttgcggaggctggttgaaacgtaatgtcattcccgagaccagctcccgttacggcaactttgacattttaagagatgaactagaagtcgttttgaaagatgtccttcaagaacccaaaactgaagatatagtagcagtgcagaaagcaaaagcattgtacaggtcttgtataaatgaatctgctattgatagcagaggtggagaacctctactcaaactgttaccagacatatatgggtggccagtagcaacagaaaactgggagcaaaaatatggtgcttcttggacagctgaaaaagctattgcacaactgaattctaaatatgggaaaaaagtccttattaatttgtttgttggcactgatgataagaattctgtgaatcatgtaattcatattgaccaacctcgacttggcctcccttctagagattactatgaatgcactggaatctataaagaggcttgtacagcatatgtggattttatgatttctgtggccagattgattcgtcaggaagaaagattgcccatcgatgaaaaccagcttgctttggaaatgaataaagttatggaattggaaaaagaaattgccaatgctacggctaaacctgaagatcgaaatgatccaatgcttctgtataacaagatgacattggcccagatccaaaataacttttcactagagatcaatgggaagccattcagctggttgaatttcacaaatgaaatcatgtcaactgtgaatattagtattacaaatgaggaagatgtggttgtttatgctccagaatatttaaccaaacttaagcccattcttaccaaatattctgccagagatcttcaaaatttaatgtcctggagattcataatggatcttgtaagcagcctcagccgaacctacaaggagtccagaaatgctttccgcaaggccctttatggtacaacctcagaaacagcaacttggagacgttgtgcaaactatgtcaatgggaatatggaaaatgctgtggggaggctttatgtggaagcagcatttgctggagagagtaaacatgtggtcgaggatttgattgcacagatccgagaagtttttattcagactttagatgacctcacttggatggatgccgagacaaaaaagagagctgaagaaaaggccttagcaattaaagaaaggatcggctatcctgatgacattgtttcaaatgataacaaactgaataatgagtacttggagttgaactacaaagaagatgaatacttcgagaacataattcaaaatttgaaattcagccaaagtaaacaactgaagaagctccgagaaaaggtggacaaagatgagtggataagtggagcagctgtagtcaatgcattttactcttcaggaagaaatcagatagtcttcccagccggcattctgcagccccccttctttagtgcccagcagtccaactcattgaactatgggggcatcggcatggtcataggacacgaaatcacccatggcttcgatgacaatggcagaaactttaacaaagatggagacctcgttgactggtggactcaacagtctgcaagtaactttaaggagcaatcccagtgcatggtgtatcagtatggaaacttttcctgggacctggcaggtggacagcaccttaatggaattaatacactgggagaaaacattgctgataatggaggtcttggtcaagcatacagagcctatcagaattatattaaaaagaatggcgaagaaaaattacttcctggacttgacctaaatcacaaacaactatttttcttgaactttgcacaggtgtggtgtggaacctataggccagagtatgcggttaactccattaaaacagatgtgcacagtccaggcaatttcaggattattgggactttgcagaactctgcagagttttcagaagcctttcactgccgcaagaattcatacatgaatccagaaaagaagtgccgggtttggtga

SEQ ID NO: 6 - DNA, 2250 bp, Homo sapiens
ggcaagtcagaaagtcagatggatataactgatatcaacactccaaagccaaagaagaaacagcgatggactccactggagatcagcctctcggtccttgtcctgctcctcaccatcatagctgtgacaatgatcgcactctatgcaacctacgatgatggtatttgcaagtcatcagactgcataaaatcagctgctcgactgatccaaaacatggatgccaccactgagccttgtacagactttttcaaatatgcttgcggaggctggttgaaacgtaatgtcattcccgagaccagctcccgttacggcaactttgacattttaagagatgaactagaagtcgttttgaaagatgtccttcaagaacccaaaactgaagatatagtagcagtgcagaaagcaaaagcattgtacaggtcttgtataaatgaatctgctattgatagcagaggtggagaacctctactcaaactgttaccagacatatatgggtggccagtagcaacagaaaactgggagcaaaaatatggtgcttcttggacagctgaaaaagctattgcacaactgaattctaaatatgggaaaaaagtccttattaatttgtttgttggcactgatgataagaattctgtgaatcatgtaattcatattgaccaacctcgacttggcctcccttctagagattactatgaatgcactggaatctataaagaggcttgtacagcatatgtggattttatgatttctgtggccagattgattcgtcaggaagaaagattgcccatcgatgaaaaccagcttgctttggaaatgaataaagttatggaattggaaaaagaaattgccaatgctacggctaaacctgaagatcgaaatgatccaatgcttctgtataacaagatgacattggcccagatccaaaataacttttcactagagatcaatgggaagccattcagctggttgaatttcacaaatgaaatcatgtcaactgtgaatattagtattacaaatgaggaagatgtggttgtttatgctccagaatatttaaccaaacttaagcccattcttaccaaatattctgccagagatcttcaaaatttaatgtcctggagattcataatggatcttgtaagcagcctcagccgaacctacaaggagtccagaaatgctttccgcaaggccctttatggtacaacctcagaaacagcaacttggagacgttgtgcaaactatgtcaatgggaatatggaaaatgctgtggggaggctttatgtggaagcagcatttgctggagagagtaaacatgtggtcgaggatttgattgcacagatccgagaagtttttattcagactttagatgacctcacttggatggatgccgagacaaaaaagagagctgaagaaaaggccttagcaattaaagaaaggatcggctatcctgatgacattgtttcaaatgataacaaactgaataatgagtacctcgagttgaactacaaagaagatgaatacttcgagaacataattcaaaatttgaaattcagccaaagtaaacaactgaagaagctccgagaaaaggtggacaaagatgagtggataagtggagcagctgtagtcaatgcattttactcttcaggaagaaatcagatagtcttcccagccggcattctgcagccccccttctttagtgcccagcagtccaactcattgaactatgggggcatcggcatggtcataggacacgaaatcacccatggcttcgatgacaatggcagaaactttaacaaagatggagacctcgttgactggtggactcaacagtctgcaagtaactttaaggagcaatcccagtgcatggtgtatcagtatggaaacttttcctgggacctggcaggtggacagcaccttaatggaattaatacactgggagaaaacattgctgataatggaggtcttggtcaagcatacagagcctatcagaattatattaaaaagaatggcgaagaaaaattacttcctggacttgacctaaatcacaaacaactatttttcttgaactttgcacaggtgtggtgtggaacctataggccagagtatgcggttaactccattaaaacagatgtgcacagtccaggcaatttcaggattattgggactttgcagaactctgcagagttttcagaagcctttcactgccgcaagaattcatacatgaatccagaaaagaagtgccgggtttggtga

SEQ ID NO: 7 - DNA, 8339 bp, Artificial Sequence
  /note=“Description of Artificial Sequence Synthetic expression vector”
acggattagaagccgccgagcgggtgacagccctccgaaggaagactctcctccgtgcgtcctcgtcctcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttatgagatttccttcaatttttactgctgttttattcgcagcatcctctgcattagctgctccagtcaacactataacagaagatgaaacggcacaaattcctgctgaagctgtcatcggttactcagatttagaaggggatttcgatgttgctgttttgccattttccaacagcacaaataacgggttattgtttatgaatactactactgccagcattgctgctaaagaagaagtggtatctctcgagaaaagagaggctgaagcttatccatatgacgtcccagactatgcttatccatatgacgtcccagactatgcttacccttacgatgtacctgattacgcaggatcctacgatgatggtatttgcaagtcatcagactgcataaaatcagctgctcgactgatccaaaacatggatgccaccactgagccttgtacagactttttcaaatatgcttgcggaggctggttgaaacgtaatgtcattcccgagaccagctcccgttacggcaactttgacattttaagagatgaactagaagtcgttttgaaagatgtccttcaagaacccaaaactgaagatatagtagcagtgcagaaagcaaaagcattgtacaggtcttgtataaatgaatctgctattgatagcagaggtggagaacctctactcaaactgttaccagacatatatgggtggccagtagcaacagaaaactgggagcaaaaatatggtgcttcttggacagctgaaaaagctattgcacaactgaattctaaatatgggaaaaaagtccttattaatttgtttgttggcactgatgataagaattctgtgaatcatgtaattcatattgaccaacctcgacttggcctcccttctagagattactatgaatgcactggaatctataaagaggcttgtacagcatatgtggattttatgatttctgtggccagattgattcgtcaggaagaaagattgcccatcgatgaaaaccagcttgctttggaaatgaataaagttatggaattggaaaaagaaattgccaatgctacggctaaacctgaagatcgaaatgatccaatgcttctgtataacaagatgacattggcccagatccaaaataacttttcactagagatcaatgggaagccattcagctggttgaatttcacaaatgaaatcatgtcaactgtgaatattagtattacaaatgaggaagatgtggttgtttatgctccagaatatttaaccaaacttaagcccattcttaccaaatattctgccagagatcttcaaaatttaatgtcctggagattcataatggatcttgtaagcagcctcagccgaacctacaaggagtccagaaatgctttccgcaaggccctttatggtacaacctcagaaacagcaacttggagacgttgtgcaaactatgtcaatgggaatatggaaaatgctgtggggaggctttatgtggaagcagcatttgctggagagagtaaacatgtggtcgaggatttgattgcacagatccgagaagtttttattcagactttagatgacctcacttggatggatgccgagacaaaaaagagagctgaagaaaaggccttagcaattaaagaaaggatcggctatcctgatgacattgtttcaaatgataacaaactgaataatgagtacttggagttgaactacaaagaagatgaatacttcgagaacataattcaaaatttgaaattcagccaaagtaaacaactgaagaagctccgagaaaaggtggacaaagatgagtggataagtggagcagctgtagtcaatgcattttactcttcaggaagaaatcagatagtcttcccagccggcattctgcagccccccttctttagtgcccagcagtccaactcattgaactatgggggcatcggcatggtcataggacacgaaatcacccatggcttcgatgacaatggcagaaactttaacaaagatggagacctcgttgactggtggactcaacagtctgcaagtaactttaaggagcaatcccagtgcatggtgtatcagtatggaaacttttcctgggacctggcaggtggacagcaccttaatggaattaatacactgggagaaaacattgctgataatggaggtcttggtcaagcatacagagcctatcagaattatattaaaaagaatggcgaagaaaaattacttcctggacttgacctaaatcacaaacaactatttttcttgaactttgcacaggtgtggtgtggaacctataggccagagtatgcggttaactccattaaaacagatgtgcacagtccaggcaatttcaggattattgggactttgcagaactctgcagagttttcagaagcctttcactgccgcaagaattcatacatgaatccagaaaagaagtgccgggtttggtgagcggccgccaccgcggtggagctccagcttttgttccctttagtgagggttaattgcgcgcttggcgtaatcatggtcatagctgtttcctgtgagatctcctaccgcatgcatctagctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccattcagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 8 - protein, 41 aa, Homo sapiens
DAEFRHDSGYEVHHQKLVFFAEDVGSNKGAIIGLMVGGVVK

SEQ ID NO: 9 - protein, 43 aa, Homo sapiens
DAEFRHDSGYEVHHQKLVFFAEDVGSNKGAIIGLMVGGVVIAK

SEQ ID NO: 10 - protein, 29 aa, Homo sapiens
SLRRSSCFGGRMDRIGAQSGLGCNSFRYK

SEQ ID NO: 11 - protein, 33 aa, Homo sapiens
SPKMVQGSGCFGRKMDRISSSSGLGCKVLRRHK

SEQ ID NO: 12 - protein, 12 aa, Homo sapiens
CDRVYIHPFHLK

SEQ ID NO: 13 - protein, 23 aa, Homo sapiens
GCSSSSLMDKESVYFCHLDIIWK

SEQ ID NO: 14 - protein, 23 aa, Homo sapiens
GSSCSSLMDKECVYFSHLDIIWK

SEQ ID NO: 15 - protein, 38 aa, Homo sapiens
CYPSKPDNPGEDAPAEDMARYYSALRHYINLITRQRYK

SEQ ID NO: 16 - protein, 15 aa, Homo sapiens
CQLYENKPRRPYILK

SEQ ID NO: 17 - protein, 31 aa, Homo sapiens
FVNQHLCGSHLVEALYLVCGERGFFYTPKTK

SEQ ID NO: 18 - protein, 11 aa, Homo sapiens
CRPPGFSPFRK

SEQ ID NO: 19 - DNA, 72 bp, Artificial Sequence
  /note=“Description of Artificial Sequence Synthetic PCR primer NEP-85A”
gacgtcccagactatgcttacccttacgatgtacctgattacgcaggatcctacgatgatggtatttgcaag

SEQ ID NO: 20 - DNA, 34 bp, Artificial Sequence
  /note=“Description of Artificial Sequence Synthetic PCR primer NEP-24”
atagtttagcggccgctcaccaaacccggcactt

SEQ ID NO: 21 - DNA, 87 bp, Artificial Sequence
  /note=“Description of Artificial Sequence Synthetic PCR primer NEP-85B”
gtatctctcgagaaaagagaggctgaagcttatccatatgacgtcccagactatgcttatccatatgacgtcccagactatgcttac

SEQ ID NO: 22 - DNA, 5856 bp, Artificial Sequence
  /note=“Description of Artificial Sequence Synthetic expression vector pYES2”
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttggtaccgagctcggatccactagtaacggccgccagtgtgctggaattctgcagatatccatcacactggcggccgctcgagcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccattcagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 23 - DNA, 6631 bp, Artificial Sequence
  /note=“Description of Artificial Sequence Synthetic expression vector pESC-URA”
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctgaattggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcagatcttatcgtcgtcatccttgtaatccatcgatactagtgcggccgccctttagtgagggttgaattcgaattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccgtaatacgactcactatagggcccgggcgtcgacatggaacagaagttgatttccgaagaagacctcgagtaagcttggtaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 24 - DNA, 6702 bp, Artificial Sequence
  /note=“Description of Artificial Sequence Synthetic expression vector p427-TEF”
caatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggagctcatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttctagaactagtggatcccccgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatagggtaataactgatataattaaattgaagctctaatttgtgagtttagtatacatgcatttacttataatacagtttctggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttgccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgaaacgtgagtcttttccttacccatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggccttggtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcc

SEQ ID NO: 25 - protein, 19 aa, Artificial Sequence
  /note=“Description of Artificial Sequence Synthetic amino acid sequence of signal peptide used in expression constructs”
MGWSCIILFLVATATGAHS

SEQ ID NO: 26 - protein, 8 aa, Artificial Sequence
  /note=“Description of Artificial Sequence Synthetic amino acid sequence of hinge region (from IgG1)”
THTCPPCP

SEQ ID NO: 27 - protein, 217 aa, Artificial Sequence
  /note=“Description of Artificial Sequence Synthetic amino acid sequence of the CH2 and CH3 domains from the Fc domain from IgG1”
APGLLGGPSVFLFPPKPKDTLMISRTPGVTCVVVDVSHGDPGVKFNWYVDGVGVHNAKTKPRGGQYNSTYRVVSVLTVLHQDWLNGKGYKCKVSNKALPAPIGKTISKAKGQPRGPQVYTLPPSRGGMTKNQVSLTCLVKGFYPSDIAVGWGSNGQPGNNYKTTPPVLDSDGSFFLYSKLTVDKSRWQQGNVFSCSVMHGALHNHYTQKSLSLSPGK

SEQ ID NO: 28 - protein, 699 aa, Homo sapiens
YDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYVTTSETATWRRCANYVNGNMMNAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFFDNGRNPNKDDDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPKNFRIIGTLQNSAEFSEAFHCRKNSYMNPEKKCRVW

SEQ ID NO: 29 - protein, 1289 aa, Artificial Sequence [sequence omitted]

SEQ ID NO: 30 - protein, 585 aa, Homo sapiens
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 31 - protein, 5 aa, Artificial Sequence
  /note=“Description of Artificial Sequence Synthetic peptide”
GGGGS

SEQ ID NO: 32 - protein, 6 aa, Artificial Sequence
  /note=“Description of Artificial Sequence Synthetic peptide”
GGGGGS

SEQ ID NO: 33 - protein, 43 aa, Homo sapiens
DAEFRHDSGYEVHHQKLVFFAEDVGSNKGAIIGLMVGGVVIAT

SEQ ID NO: 34 - protein, 4 aa, Artificial Sequence
  /note=“Description of Artificial Sequence Synthetic peptide”
GGGG

SEQ ID NO: 35 - protein, 5 aa, Artificial Sequence
  /note=“Description of Artificial Sequence Synthetic peptide”
GGGGG

SEQ ID NO: 36 - protein, 10 aa, Artificial Sequence
  /note=“Description of Artificial Sequence Synthetic 10xHis tag”
HHHHHHHHHH

SEQ ID NO: 37 - protein, 40 aa, Artificial Sequence
  /note=“Description of Artificial Sequence Synthetic polypeptide”
DAEFRHDSGYEVHHQKLVFFAEDVGSNKGAIIGLMVGGVV

SEQ ID NO: 38 - protein, 13 aa, Artificial Sequence
  /note=“Description of Artificial Sequence Synthetic peptide”
QLYENKPRRPYIL

SEQ ID NO: 39 - protein, 28 aa, Artificial Sequence
  /note=“Description of Artificial Sequence Synthetic peptide”
SLRRSSCFGGRMDRIGAQSGLGCNSFRY

SEQ ID NO: 40 - protein, 21 aa, Artificial Sequence
  /note=“Description of Artificial Sequence Synthetic peptide”
CSSSSLMDKESVYFCHLDIIW

SEQ ID NO: 41 - protein, 21 aa, Artificial Sequence
  /note=“Description of Artificial Sequence Synthetic peptide”
SSCSSLMDKECVYFSHLDIIW

SEQ ID NO: 42 - protein, 31 aa, Artificial Sequence
  /note=“Description of Artificial Sequence Synthetic polypeptide”
HAEGTFTSDVSSYLEGQAAKEFIAWLVKGRG

SEQ ID NO: 43 - protein, 10 aa, Artificial Sequence
  /note=“Description of Artificial Sequence Synthetic peptide”
DRVYIHPFHL

SEQ ID NO: 44 - protein, 9 aa, Artificial Sequence
  /note=“Description of Artificial Sequence Synthetic peptide”
RPPGFSPFR

SEQ ID NO: 45 - protein, 42 aa, Artificial Sequence
  /note=“Description of Artificial Sequence Synthetic polypeptide”
YAEGTFISDYSIAMDKIHQQDFVNWLLAQKGKKNDWKHNITQ

SEQ ID NO: 46 - protein, 28 aa, Artificial Sequence
  /note=“Description of Artificial Sequence Synthetic peptide”
SANSNPAMAPRERKAGCKNFFWKTFTSC

SEQ ID NO: 47 - protein, 29 aa, Artificial Sequence
  /note=“Description of Artificial Sequence Synthetic peptide”
HSQGTFTSDYSKYLDSRRAQDFVQWLMNT

SEQ ID NO: 48 - protein, 749 aa, Artificial Sequence; identical to SEQ ID NO: 1
  /note=“Description of Artificial Sequence Synthetic polypeptide”

SEQ ID NO: 49 - protein, 749 aa, Artificial Sequence
  /note=”Description of Artificial Sequence Synthetic polypeptide“
GKSESQMDITDINTPKPKKKQRWTPLEISLSVLVLLLTIIAVTMIALYATYDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYXTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPXNFRIIGTLQNSAEFSEAFHCRKNSYMNPEKKCRVW

SEQ ID NO: 50 - protein, 749 aa, Artificial Sequence; identical to SEQ ID NO: 1
  /note=”Description of Artificial Sequence Synthetic polypeptide“